(19) United States
(12) Patent Application Publication
(10) Pub. No.: US 2025/0368608 A1
(43) Pub. Date: Dec. 4, 2025

(54) SELECTIVE HDAC6 INHIBITOR

(71) Applicants: ROYAL COLLEGE OF SURGEONS IN IRELAND, Dublin (IE); DANA-FARBER CANCER INSTITUTE, INC., Boston, MA (US)

(72) Inventors: Triona Ni Chonghaile, Dublin (IE); Catriona Dowling, Dublin (IE); Daniel Alencar Rodrigues, Dublin (IE); Anthony Letai, Boston, MA (US)

(73) Assignees: ROYAL COLLEGE OF SURGEONS IN IRELAND, Dublin (IE); DANA-FARBER CANCER INSTITUTE, INC., Boston, MA (US)

(21) Appl. No.: 18/257,677

(22) PCT Filed: Dec. 15, 2021
(86) PCT No.: PCT/EP2021/086038

§ 371 Date: Jun. 15, 2023

(51) Int. Cl. C07D 235/28; A61K 31/4184; A61K 31/454; A61K 31/496; A61K 31/5377; A61K 31/541; A61K 45/06; A61P 35/00; C07D 233/42; C07D 233/84; C07D 401/12; C07D 403/12

(52) U.S. Cl. CPC C07D 235/28; A61K 31/4184; A61K 31/454; A61K 31/496; A61K 31/5377; A61K 31/541; A61K 45/06; A61P 35/00; C07D 233/42; C07D 233/84; C07D 401/12; C07D 403/12

(57) ABSTRACT

A compound, which is according to formula I or is a pharmaceutically acceptable salt, solvate, ester or pro-drug thereof, for use as a medicament,

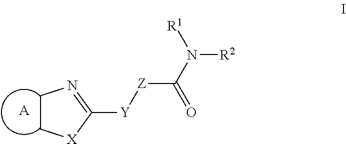

wherein
either (i) NR¹R² together forms a 5- or 6-membered heterocyclic ring or (ii) R¹ and R² are independently selected from H and C1 to C12 alkyl;
A is a non-aromatic ring, an aromatic ring or a double bond;
X is NR³, S or O, where R³ is selected from H and C1 to C12 alkyl;
Y is S, NR⁴, CR⁴R⁵, or O, where R⁴ and R⁵ are independently selected from H and C1 to C12 alkyl; and
Z is (CR⁶R⁷)ₙ where R⁶ and R⁷ are independently selected from H and C1 to C12 alkyl and n is an integer from 1 to 6.

The compounds of formula I are particularly useful for the treatment of cancer, neurodegenerative diseases and/or inflammation.

### FIELD OF THE INVENTION

The invention relates to compounds for use as a medicament, including in the treatment of cancer, neurodegenerative diseases and inflammation.

### BACKGROUND TO THE INVENTION

Triple negative breast cancer (TNBC) is a highly aggressive subtype of breast cancer that lacks expression of estrogen receptor (ER), progesterone receptor (PR) and human epidermal growth factor 2 (HER2). Numerous ‘omic’ studies have tried to molecularly characterize the heterogeneous disease and identify ‘driver’ mutations to therapeutically target. The standard of care, for both recently diagnosed and patients with advanced-disease, is cytotoxic chemotherapy and targeted therapies are not routinely used in the treatment of TNBC. While chemotherapy is effective in a subset of patients, there is a large proportion of patients (60-70%) that are refractory to chemotherapy and have poorer survival. Novel therapeutic strategies are urgently required for TNBC patients with chemoresistant disease.

Histone deacetylase inhibitors (HDACis) have emerged as a powerful class of small-molecule therapeutics acting through the regulation of the acetylation states of histone proteins (a form of epigenetic modulation) and other non-histone protein targets. A number of structurally distinct HDACis have been approved including SAHA (Suberoylanilide Hydroxamic Acid, a.k.a. vorinsotat), romidepsin (FK228), belinostat, panobinostat, and Chidamide. The current HDACis have serious limitations, for example, lack of specificity, ineffectively low concentrations in solid tumors, and/or cardiac toxicity.

### SUMMARY OF THE INVENTION

According to a first aspect of the invention there is provided a compound which is according to formula I or is a pharmaceutically acceptable salt, solvate, ester or pro-drug thereof, for use as a medicament,

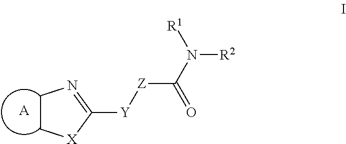

wherein
either (i) NR¹R² together forms a 5- or 6-membered heterocyclic ring or (ii) R¹ and R² are independently selected from H and C1 to C12 alkyl;
A is a non-aromatic ring, an aromatic ring or a double bond;
X is NR³, S or O, where R³ is selected from H and C1 to C12 alkyl;
Y is S, NR⁴, CR⁴R⁵, or O, where R⁴ and R⁵ are independently selected from H and C1 to C12 alkyl; and
Z is (CR⁶R⁷)ₙ where R⁶ and R⁷ are independently selected from H and C1 to C12 alkyl and n is an integer from 1 to 6.

According to a second aspect of the invention, there is provided the compound of the first aspect for use in the treatment of cancer, neurodegenerative diseases and/or inflammation.

According to a third aspect of the invention there is provided a pharmaceutical composition comprising a compound which is according to formula I or a pharmaceutically acceptable salt, solvate, ester or pro-drug thereof, and a pharmaceutically acceptable carrier.

According to a fourth aspect of the invention, there is provided a method of medical treatment comprising administering to a subject in need thereof an effective amount of a compound which is according to formula I or a pharmaceutically acceptable salt, solvate, ester or pro-drug thereof.

In particular, the invention resides in a method for the treatment of cancer, neurodegenerative diseases and/or inflammation.

The invention also resides in specific compounds according to formula I or pharmaceutically acceptable salts, solvates, esters or pro-drugs thereof. In particular, compounds S2 to S7, S10, S17, S19, S20, S22 to S28, C1 to C7, C15 to C28, N1 to N8, N10, N13, N15 to N18, N20 to N28. Histone deacetylase (HDAC) are a family of enzymes that modulate their substrates by removing the acetyl group from lysine residues. Using an in vitro HDAC inhibitor screen, the inventors have demonstrated that a known HDAC inhibitor (SAHA) inhibits the activity of HDAC 1, 2, 6, 7 and 8 (FIG. 4, left). In contrast, a compound of formula I (BAS-2, compound 1) inhibited only the isozyme HDAC6 with an IC50 of 76 nM (FIG. 4, right). As such, BAS-2 is a selective HDAC6 inhibitor.

The examples demonstrate the properties of the compounds of the invention. In particular, the in vivo efficacy of BAS-2 was assessed and found to reduce tumor volume and weight (e.g. FIG. 8).

### DETAILED DESCRIPTION OF THE INVENTION

The invention is concerned with the use of a compound according to formula I as a medicament.

References to the compound also refer to a pharmaceutically acceptable salt, solvate ester or pro-drug thereof, where the context allows.

A may be a non-aromatic ring or an aromatic ring. It will be appreciated that the ring is fused; sharing two carbon atoms with the five membered nitrogen containing heterocycle.

The term “non-aromatic ring” as used herein refers to a saturated carbocyclic ring system having from 6 to 30 ring carbon atoms.

The term “aromatic ring” as used herein refers to an aromatic carbocyclic ring system having from 6 to 30 ring carbon atoms. For instance, an aromatic ring may have from 6 to 16 ring carbon atoms, e.g. from 6 to 10 ring carbon atoms. Typically, the aromatic ring is a monocyclic aromatic ring system. For example, A may be a benzene ring and X may be nitrogen to provide a benzimidazole. However A could be a polycyclic ring system having two or more rings, at least one of which is aromatic.

When present, the non-aromatic ring or the aromatic ring may be substituted with a C1 to C12 alkyl group, a C1 to C12 alkenyl group, a C1 to C12 alkoxy group, a carboxy group, a hydroxy group, and/or a halo group (e.g. F, Cl, Br or I). When present, the non-aromatic ring or the aromatic ring may be unsubstituted or substituted with C1 to C6 alkyl.

A may be a double bond. If so, the five-membered nitrogen containing heterocycle is not fused to another ring.

X may be NR³, where R³ is selected from H and C1 to C12 alkyl. For example X may be NH or NCH₃. As such, the five membered heterocycle comprises 2 nitrogen atoms (an imidazole).

X may be S; the five membered heterocycle comprises 1 nitrogen atom and 1 S atom (a thiazole).

X may be O; the five membered heterocycle comprises 1 nitrogen atom and 1 O atom (an oxazole).

—Y—Z— acts as a linker between the five-membered heterocycle and the amide group.

Y may be S. For example, —Y— may be —S— and —Z— may be —CH₂—.

Y may be NR⁴. For example, —Y— may be —NH— or —NCH₃—.

Y may be CR⁴R⁵ For example, —Y— may be —CH₂— or —CHCH₂—.

Y may be O.

Z is (CR⁶R⁷)ₙ where R⁶ and R⁷ are independently selected from H and C1 to C12 alkyl and n is an integer from 1 to 6: 1, 2, 3, 4, 5 or 6. For example, Z may be (CH₂)ₙ where n is 1, 2 or 3.

Z may be —CH₂— i.e. each of R⁶ and R⁷ is H and n is 1. As such, the compound may be according to formula II.

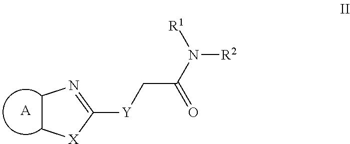

When present, an alkyl group (e.g. R¹ to R⁷) may comprise from 1 to 12 carbon atoms (e.g. from 1 to 10, 2 to 8 or 2 to 4 carbon atoms), such as a methyl, ethyl, propyl, or butyl group. An alkyl group may be a straight or branched chain alkyl moiety or a cyclic moiety.

For example, when present, each of R³ to R⁷ may be H or CH₃.

In one embodiment (i) R⁶ is H; R⁷ is H; and/or (iii) n is 1 or 2.

NR¹R² may form a 5- or 6-membered heterocyclic ring, i.e. a heterocycle comprising at least one nitrogen atom. When present the 5- or 6-membered heterocyclic ring may comprise just one nitrogen atom (no additional heteroatoms). When present the 5- or 6-membered heterocyclic ring may comprise at least one additional heteroatom selected from N. S and 0.

When present, the 5- or 6-membered heterocyclic ring may comprise a morpholine group, a thiomorpholine group, a piperidine group, a piperazine group, an oxazepane group or a thiazepane group.

The 5- or 6-membered heterocyclic ring may be substituted or unsubstituted. For example, the 5- or 6-membered heterocyclic ring may be substituted with a C1 to C12 alkyl group, a C1 to C12 alkenyl group, a C1 to C12 alkoxy group, a carboxy group, a hydroxy group, and/or a halo group (e.g. F, Cl, Br or I).

NR¹R² may form a morpholine group; A may be a non-aromatic ring; X may be NR³; where R³ is selected from H and C1 to C12 alkyl; Y may be S; and/or Z may be —CH₂—.

The compound of formula I may be BAS-2 (1)

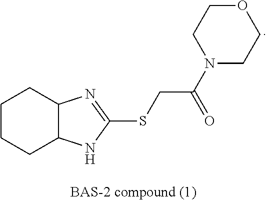 [SMILES: O=C(CSC1=NC2CCCCC2N1)N1CCOCC1]

The examples demonstrate that BAS-2 (1) is a selective HDAC6 inhibitor with IC₅₀ of 76 nM (>250 fold selectivity).

It will be appreciated that BAS-2 is a compound of formula I where NR¹R² forms a 6-membered heterocyclic ring comprising a N atom an O atom (a morpholine group); A is a non-aromatic 6-membered ring; X is NR³ (NH); Y is S; and Z is a C1 alkyl group with n=1 (CH₂).

BAS-2 (1) can be viewed as having three subunits: an imidazole, a linker (—S—CH₂)—) and an amide.

The table below provides examples of compounds of formula I where —Y—Z— is —S—CH₂— together with the results of a search from the Scifinder database. These compounds are described as the sulphur series (S). The synthesis of compounds of S series is described in the examples (TTC-01 to TTC-41).

3a,4,5,6,7,7a-hexahydro-1H-benzo[d]imidazole Series (S)

| | |
|---|---|
| 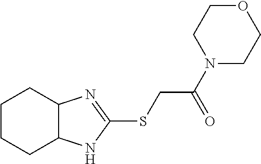 [SMILES: O=C(CSC1=NC2CCCCC2N1)N1CCOCC1] | (1) |
| BAS-2 | |
| Scifinder: CAS: 447410-08-4 MW: 283.39 | |
| (TTC-01 and TTC-02) | |
| 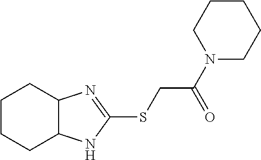 [SMILES: O=C(CSC1=NC2CCCCC2N1)N1CCCCC1] | (S2) |
| (TTC-03; TTC-10) | |
| 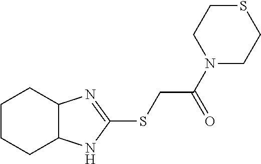 [SMILES: O=C(CSC1=NC2CCCCC2N1)N1CCSCC1] | (S3) |
| (TTC-05; TTC-11) | |
| 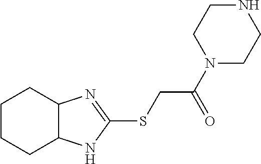 [SMILES: O=C(CSC1=NC2CCCCC2N1)N1CCNCC1] | (S4) |
| 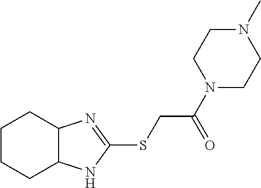 [SMILES: CN1CCN(C(=O)CSC2=NC3CCCCC3N2)CC1] | (S5) |
| 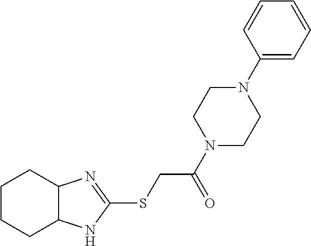 [SMILES: O=C(CSC1=NC2CCCCC2N1)N1CCN(c2ccccc2)CC1] | (S6) |
| (TTC-07; TTC-13) | |
| 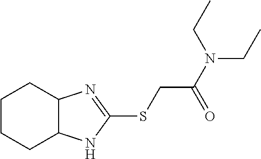 [SMILES: CCN(CC)C(=O)CSC1=NC2CCCCC2N1] | (S7) |
| (TTC-04) | |

Benzimidazole Series (S)

| | |
|---|---|
| 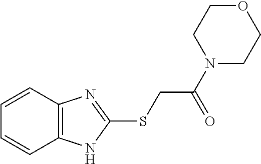 [SMILES: O=C(CSc1nc2ccccc2[nH]1)N1CCOCC1] | (S8) |
| Scifinder: | |
| CAS: 30065-31-7 | |
| MW: 277.34 | |
| TTC-21 | |
| 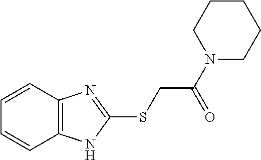 [SMILES: O=C(CSc1nc2ccccc2[nH]1)N1CCCCC1] | (S9) |
| Scifinder: | |
| CAS: 337488-07-0 | |
| MW: 275.37 | |
| References: | |
| DOI: 10.1021/jo01107a004 (1955); | |
| US20090163545 | |
| 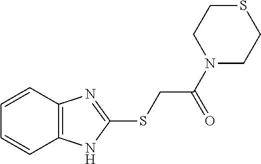 [SMILES: O=C(CSc1nc2ccccc2[nH]1)N1CCSCC1] | (S10) |
| 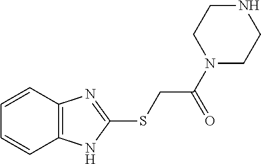 [SMILES: O=C(CSc1nc2ccccc2[nH]1)N1CCNCC1] | (S11) |
| Scifinder: | |
| CAS: 925213-81-6 | |
| MW: 276.36 | |
| -References: | |
| DOI: 10.1016/j.ejmech.2006.07.005; | |
| 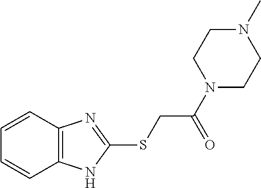 [SMILES: CN1CCN(C(=O)CSc2nc3ccccc3[nH]2)CC1] | (S12) |
| Scifinder: | |
| CAS: 321975-06-8 | |
| MW: 290.38 | |
| Acta Poloniae Pharmaceutica, 1985, 42, | |
| 19-23 refers to the 2HCl salt | |
| 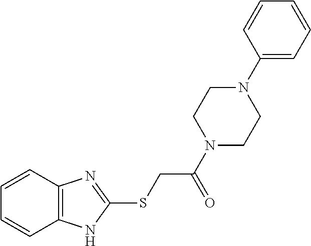 [SMILES: O=C(CSc1nc2ccccc2[nH]1)N1CCN(c2ccccc2)CC1] | (S13) |
| Scifinder: | |
| CAS: 392714-60-2 | |
| MW: 352.45 | |
| References: | |
| Bulletin of the Faculty of Pharmacy (Cairo | |
| University), 2002, 40 (1), 7-13; | |
| US20090163545 | |
| 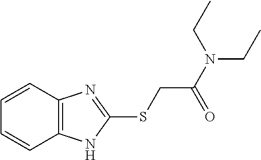 [SMILES: CCN(CC)C(=O)CSc1nc2ccccc2[nH]1] | (S14) |
| Scifinder: | |
| CAS: 51171-38-1 | |
| MW: 263.36 | |
| -References: | |
| Rozprawy Wydzialu 3: Nauk Matematyczno-Przyrodniczych, | |
| Gdanskie Towarzystwo Naukowe, 1973, 9, 135-140 | |

Imidazole Series (S)

| | |
|---|---|
| 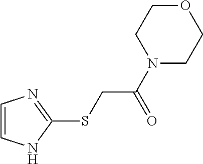 [SMILES: O=C(CSc1ncc[nH]1)N1CCOCC1] | (S15) |
| Scifinder: | |
| CAS: 1394756-26-3 | |
| MW: 227.28 | |
| TTC-22 | |
| 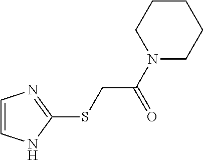 [SMILES: O=C(CSc1ncc[nH]1)N1CCCCC1] | (S16) |
| Scifinder: | |
| CAS: 1908350-34-5 | |
| MW: 225.31 | |
| 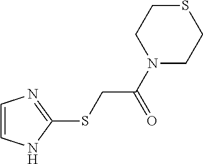 [SMILES: O=C(CSc1ncc[nH]1)N1CCSCC1] | (S17) |
| 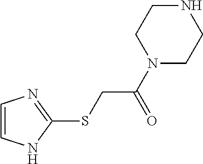 [SMILES: O=C(CSc1ncc[nH]1)N1CCNCC1] | (S18) |
| Scifinder: | |
| CAS: 1697328-81-6 | |
| MW: 226.30 | |
| 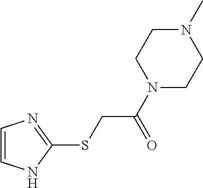 [SMILES: CN1CCN(C(=O)CSc2ncc[nH]2)CC1] | (S19) |
| 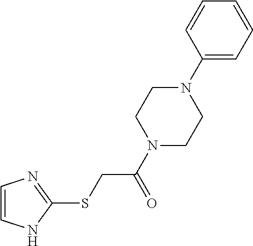 [SMILES: O=C(CSc1ncc[nH]1)N1CCN(c2ccccc2)CC1] | (S20) |
| 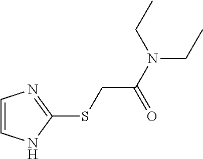 | (S21) |
| Scifinder: | |
| CAS: 1394723-12-6 | |
| MW: 213.30 | |

4,5,6,7-tetrahydro-1H-benzo[d]imidazole Series (S)

| | |
|---|---|
| 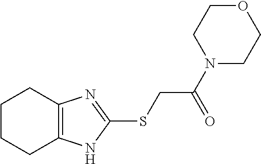 [SMILES: O=C(CSc1nc2c([nH]1)CCCC2)N1CCOCC1] | (S22) |
| TTC-23 | |
| 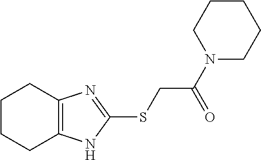 [SMILES: O=C(CSc1nc2c([nH]1)CCCC2)N1CCCCC1] | (S23) |
| 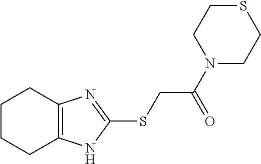 [SMILES: O=C(CSc1nc2c([nH]1)CCCC2)N1CCSCC1] | (S24) |
| 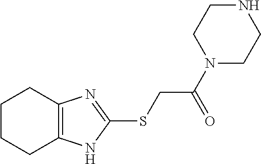 [SMILES: O=C(CSc1nc2c([nH]1)CCCC2)N1CCNCC1] | (S25) |
| 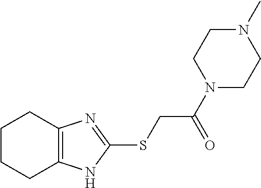 [SMILES: CN1CCN(C(=O)CSc2nc3c([nH]2)CCCC3)CC1] | (S26) |
| 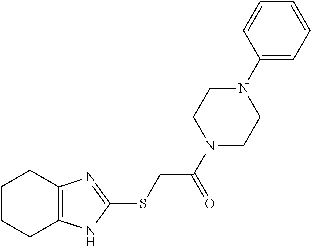 [SMILES: O=C(CSc1nc2c([nH]1)CCCC2)N1CCN(c2ccccc2)CC1] | (S27) |
| 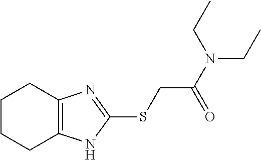 [SMILES: CCN(CC)C(=O)CSc1nc2c([nH]1)CCCC2] | (S28) |

The table below provides examples of compounds of formula I where —Y—Z— is —CH₂— CH₂— together with the results of a search from the Scifinder database. These compounds are described as the carbon series (C).

(3aR,7aS)-3a,4,5,6,7,7a-hexahydro-1H-benzo[d]imidazole and
(3aS,7aS)-3a,4,5,6,7,7a-hexahydro-1H-benzo[d]imidazole Series (C)

| | |
|---|---|
| 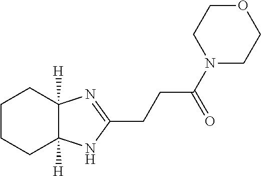 [SMILES: O=C(CCC1=N[C@@H]2CCCC[C@@H]2N1)N1CCOCC1] | (C1) |
| 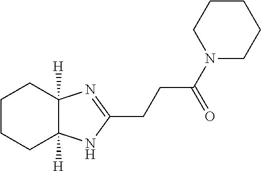 [SMILES: O=C(CCC1=N[C@@H]2CCCC[C@@H]2N1)N1CCCCC1] | (C2) |
| 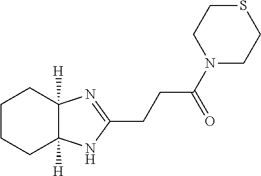 [SMILES: O=C(CCC1=N[C@@H]2CCCC[C@@H]2N1)N1CCSCC1] | (C3) |
| 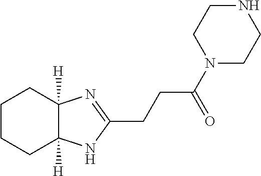 [SMILES: O=C(CCC1=N[C@@H]2CCCC[C@@H]2N1)N1CCNCC1] | (C4) |
| 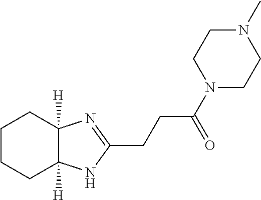 [SMILES: CN1CCN(C(=O)CCC2=N[C@@H]3CCCC[C@@H]3N2)CC1] | (C5) |
| 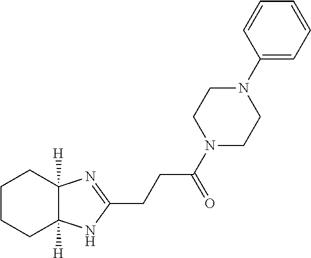 [SMILES: O=C(CCC1=N[C@@H]2CCCC[C@@H]2N1)N1CCN(c2ccccc2)CC1] | (C6) |
| 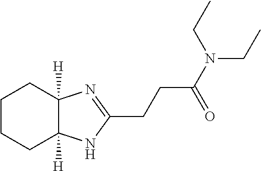 [SMILES: CCN(CC)C(=O)CCC1=N[C@@H]2CCCC[C@@H]2N1] | (C7) |

Benzimidazole Series (C)

| | |
|---|---|
| 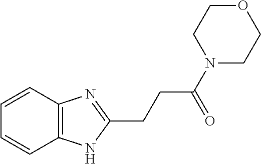 [SMILES: O=C(CCc1nc2ccccc2[nH]1)N1CCOCC1] | (C8) |
| Scifinder: | |
| CAS: 106273-15-8 | |
| References: | |
| Angewandte Chemie (1961), 73, 64 | |
| Tohoku Yakka Daigaku Kiyo (1960), 7, | |
| 95-102. | |
| 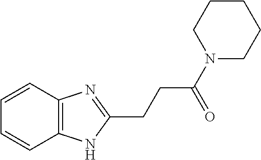 [SMILES: O=C(CCc1nc2ccccc2[nH]1)N1CCCCC1] | (C9) |
| Scifinder: | |
| CAS: 100950-03-6 | |
| MW: 275.33 | |
| References: | |
| Angewandte Chemie (1961), 73, 64 | |
| Tohoku Yakka Daigaku Kiyo (1960), 7, 95-102. | |
| Journal of the Indian Chemical Society | |
| (2013), 90(10), 1737-1748. | |
| 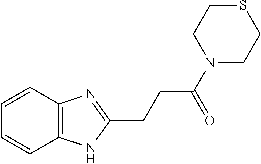 [SMILES: O=C(CCc1nc2ccccc2[nH]1)N1CCSCC1] | (C10) |
| Scifinder: | |
| CAS: 2460345-38-2 | |
| MW: 275.37 | |
| 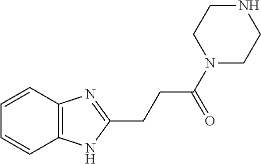 [SMILES: O=C(CCc1nc2ccccc2[nH]1)N1CCNCC1] | (C11) |
| Scifinder: | |
| CAS: 1606768-08-4 | |
| MW: 258.32 | |
| 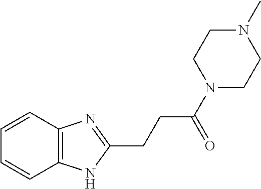 [SMILES: CN1CCN(C(=O)CCc2nc3ccccc3[nH]2)CC1] | (C12) |
| Scifinder: | |
| CAS: 1455577-84-0 | |
| MW: 272.35 | |
| 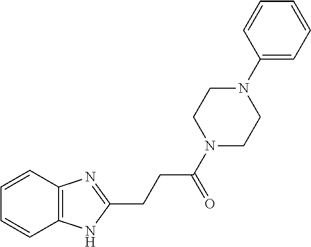 [SMILES: O=C(CCc1nc2ccccc2[nH]1)N1CCN(c2ccccc2)CC1] | (C13) |
| Scifinder: | |
| CAS: 900326-08-1 | |
| MW: 334.41 | |
| 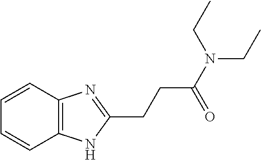 [SMILES: CCN(CC)C(=O)CCc1nc2ccccc2[nH]1] | (C14) |
| Scifinder: | |
| CAS: 101116-43-2 | |
| MW: 245.32 | |
| References: | |
| Angewandte Chemie (1961), 73, 64 | |
| Tohoku Yakka Daigaku Kiyo (1960), 7, 95-102. | |

Imidazole Series (C)

| | |
|---|---|
| 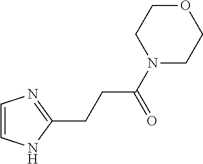 [SMILES: O=C(CCc1ncc[nH]1)N1CCOCC1] | (C15) |
| 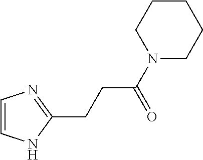 [SMILES: O=C(CCc1ncc[nH]1)N1CCCCC1] | (C16) |
| 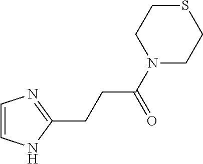 [SMILES: O=C(CCc1ncc[nH]1)N1CCSCC1] | (C17) |
| 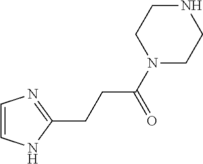 [SMILES: O=C(CCc1ncc[nH]1)N1CCNCC1] | (C18) |
| 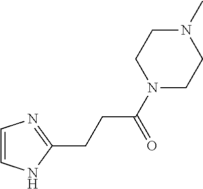 [SMILES: CN1CCN(C(=O)CCc2ncc[nH]2)CC1] | (C19) |
| 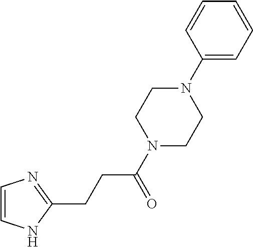 [SMILES: O=C(CCc1ncc[nH]1)N1CCN(c2ccccc2)CC1] | (C20) |
| 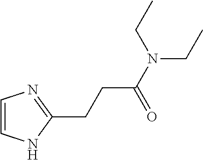 | (C21) |

4,5,6,7-tetrahydro-1H-benzo[d]imidazole Series (C)

| | |
|---|---|
| 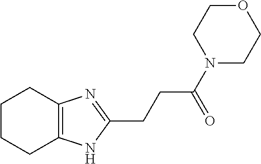 [SMILES: O=C(CCc1nc2c([nH]1)CCCC2)N1CCOCC1] | (C22) |
| 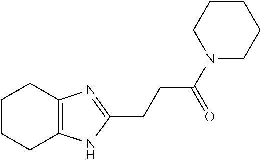 [SMILES: O=C(CCc1nc2c([nH]1)CCCC2)N1CCCCC1] | (C23) |
| 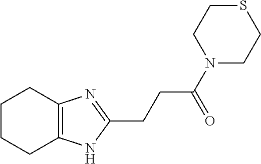 [SMILES: O=C(CCc1nc2c([nH]1)CCCC2)N1CCSCC1] | (C24) |
| 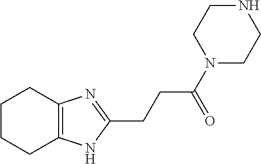 [SMILES: O=C(CCc1nc2c([nH]1)CCCC2)N1CCNCC1] | (C25) |
| 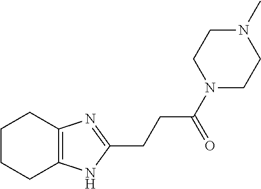 [SMILES: CN1CCN(C(=O)CCc2nc3c([nH]2)CCCC3)CC1] | (C26) |
| 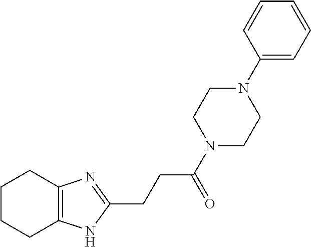 [SMILES: O=C(CCc1nc2c([nH]1)CCCC2)N1CCN(c2ccccc2)CC1] | (C27) |
| 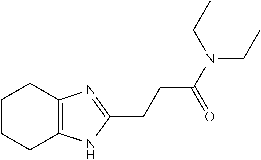 [SMILES: CCN(CC)C(=O)CCc1nc2c([nH]1)CCCC2] | (C28) |

The table below provides examples of compounds of formula I where —Y—Z— is —NH—CH₂— together with the results of a search from the Scifinder database. These compounds are described as the nitrogen series (N).

(3aR,7aS)-3a,4,5,6,7,7a-hexahydro-1H-benzo[d]imidazole and
(3aS,7aS)-3a,4,5,6,7,7a-hexahydro-1H-benzo[d]imidazole Series (N)

| | |
|---|---|
| 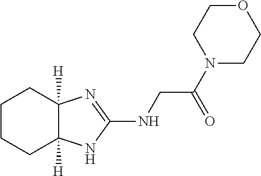 [SMILES: O=C(CNC1=N[C@@H]2CCCC[C@@H]2N1)N1CCOCC1] | (N1) |
| 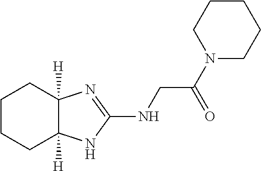 [SMILES: O=C(CNC1=N[C@@H]2CCCC[C@@H]2N1)N1CCCCC1] | (N2) |
| 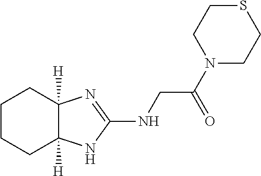 [SMILES: O=C(CNC1=N[C@@H]2CCCC[C@@H]2N1)N1CCSCC1] | (N3) |
| 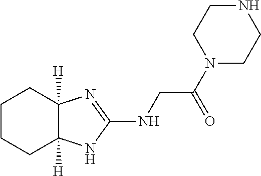 [SMILES: O=C(CNC1=N[C@@H]2CCCC[C@@H]2N1)N1CCNCC1] | (N4) |
| 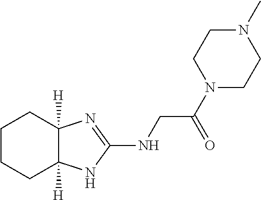 [SMILES: CN1CCN(C(=O)CNC2=N[C@@H]3CCCC[C@@H]3N2)CC1] | (N5) |
| 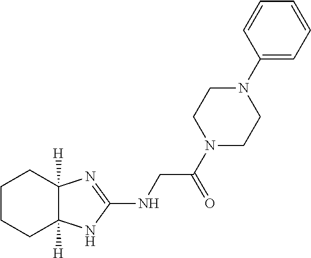 [SMILES: O=C(CNC1=N[C@@H]2CCCC[C@@H]2N1)N1CCN(c2ccccc2)CC1] | (N6) |
| 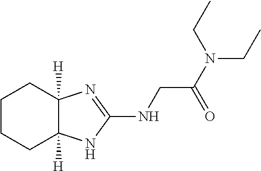 [SMILES: CCN(CC)C(=O)CNC1=N[C@@H]2CCCC[C@@H]2N1] | (N7) |

Benzimidazole Series (N)

| | |
|---|---|
| 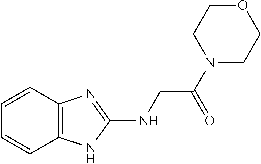 [SMILES: O=C(CNc1nc2ccccc2[nH]1)N1CCOCC1] | (N8) |
| 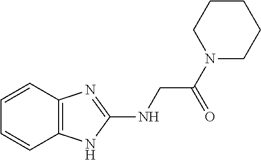 [SMILES: O=C(CNc1nc2ccccc2[nH]1)N1CCCCC1] | (N9) |
| Scifinder: | |
| CAS: 1710177-83-5 | |
| MW: 258.32 | |
| 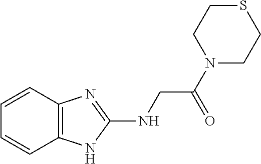 [SMILES: O=C(CNc1nc2ccccc2[nH]1)N1CCSCC1] | (N10) |
| 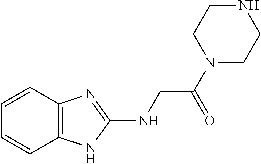 [SMILES: O=C(CNc1nc2ccccc2[nH]1)N1CCNCC1] | (N11) |
| Scifinder: | |
| CAS: 1179706-53-6 | |
| MW: 259.31 | |
| 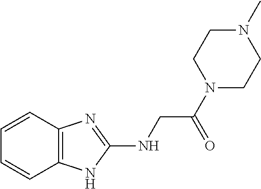 [SMILES: CN1CCN(C(=O)CNc2nc3ccccc3[nH]2)CC1] | (N12) |
| Scifinder: | |
| CAS: 114649-99-9 | |
| MW: 273.33 | |
| References: | |
| Indian Journal of Chemistry, Section B: | |
| Organic Chemistry Including Medicinal | |
| Chemistry, 1987, 26B (8), 752-756. | |
| 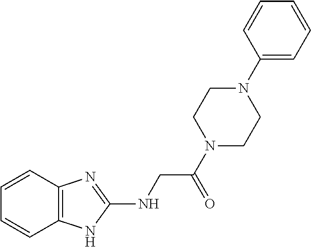 [SMILES: O=C(CNc1nc2ccccc2[nH]1)N1CCN(c2ccccc2)CC1] | (N13) |
| 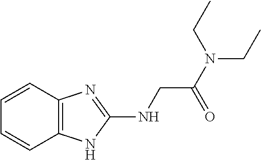 [SMILES: CCN(CC)C(=O)CNc1nc2ccccc2[nH]1] | (N14) |
| Scifinder: | |
| CAS: 1708000-39-8 | |
| MW: 246.31 | |

Imidazole Series (N)

| | |
|---|---|
| 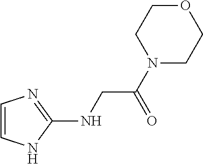 [SMILES: O=C(CNc1ncc[nH]1)N1CCOCC1] | (N15) |
| 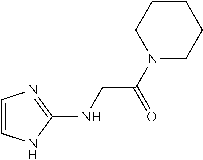 [SMILES: O=C(CNc1ncc[nH]1)N1CCCCC1] | (N16) |
| 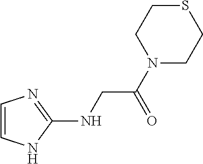 [SMILES: O=C(CNc1ncc[nH]1)N1CCSCC1] | (N17) |
| 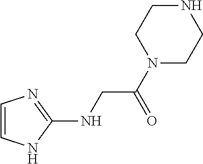 [SMILES: O=C(CNc1ncc[nH]1)N1CCNCC1] | (N18) |
| 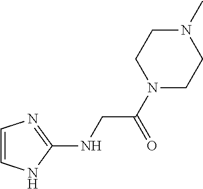 [SMILES: CN1CCN(C(=O)CNc2ncc[nH]2)CC1] | (N19) |
| Scifinder: | |
| CAS: 2128878-34-0 | |
| MW: 222.29 | |
| 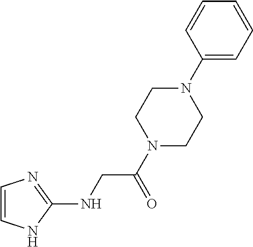 [SMILES: O=C(CNc1ncc[nH]1)N1CCN(c2ccccc2)CC1] | (N20) |
| 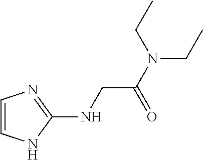 | (N21) |

4,5,6,7-tetrahydro-1H-benzo[d]imidazole Series (N)

| | |
|---|---|
| 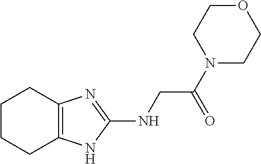 [SMILES: O=C(CNc1nc2c([nH]1)CCCC2)N1CCOCC1] | (N22) |
| 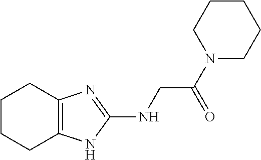 [SMILES: O=C(CNc1nc2c([nH]1)CCCC2)N1CCCCC1] | (N23) |
| 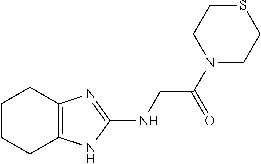 [SMILES: O=C(CNc1nc2c([nH]1)CCCC2)N1CCSCC1] | (N24) |
| 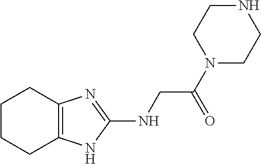 [SMILES: O=C(CNc1nc2c([nH]1)CCCC2)N1CCNCC1] | (N25) |
| 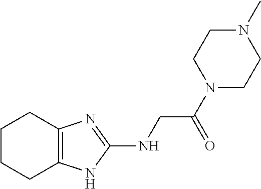 [SMILES: CN1CCN(C(=O)CNc2nc3c([nH]2)CCCC3)CC1] | (N26) |
| 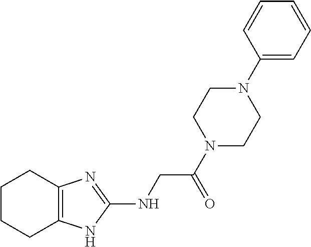 [SMILES: O=C(CNc1nc2c([nH]1)CCCC2)N1CCN(c2ccccc2)CC1] | (N27) |
| 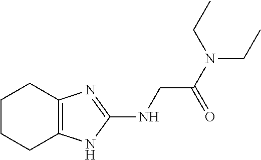 [SMILES: CCN(CC)C(=O)CNc1nc2c([nH]1)CCCC2] | (N28) |

### Salts

It may be convenient or desirable to prepare, purify, and/or handle a corresponding pharmaceutically acceptable salt of the compound. Examples of pharmaceutically acceptable salts are discussed in Berge et al, 1977, “Pharmaceutically Acceptable Salts,” J. Pharm. ScL Vol. 66, pp. 1-19. In one embodiment, the compound of formula (I) is provided in the form of a salt of an organic or mineral acid. In a particular embodiment the compound of formula I is provided in the form of a salt of a strong acid, such as HCl, HBr, HI or a sulfonic acid.

If the compound is cationic, or has a functional group which may be cationic (e.g., —NH₂ may be —NH₃⁺), then a salt may be formed with a suitable anion. Examples of suitable inorganic anions include, but are not limited to, those derived from the following inorganic acids: hydrochloric, hydrobromic, hydroiodic, sulfuric, sulfurous, nitric, nitrous, phosphoric, and phosphorous.

Examples of suitable organic anions include, but are not limited to, those derived from the following organic acids: 2-acetyoxybenzoic, acetic, ascorbic, aspartic, benzoic, camphorsulfonic, cinnamic, citric, edetic, ethanedisulfonic, ethanesulfonic, fumaric, glucheptonic, gluconic, glutamic, glycolic, hydroxymaleic, hydroxynaphthalene carboxylic, isethionic, lactic, lactobionic, lauric, maleic, malic, methanesulfonic, mucic, oleic, oxalic, palmitic, pamoic, pantothenic, phenylacetic, phenylsulfonic, propionic, pyruvic, salicylic, stearic, succinic, sulfanilic, tartaric, toluenesulfonic, and valeric. Examples of suitable polymeric organic anions include, but are not limited to, those derived from the following polymeric acids: tannic acid, carboxymethyl cellulose.

Alternatively, if the compound is anionic, or has a functional group which may be anionic, then a salt may be formed with a suitable cation. Examples of suitable inorganic cations include, but are not limited to, alkali metal ions such as Na⁺ and K⁺, alkaline earth cations such as Ca²⁺ and Mg²⁺, and other cations such as Al³⁺. Examples of suitable organic cations include, but are not limited to, ammonium ion (i.e., NH₄⁺) and substituted ammonium ions (e.g., NH₃R⁺, NH₂R²⁺, NHR₃⁺, NR₄⁺). Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

Unless otherwise specified, a reference to a particular compound also includes salt forms thereof.

In particular, the invention resides in each of the following:
TTC-01—(±)-trans-2-((2-morpholino-2-oxoethyl)thio)-3a,4,5,6,7,7a-hexahydro-1H-benzo[d]imidazol-3-ium chloride
TTC-02—cis-2-((2-morpholino-2-oxoethyl)thio)-3a,4,5,6,7,7a-hexahydro-1H-benzo[d]imidazol-3-ium chloride
TTC-03—(±)-trans-2-((2-oxo-2-(piperidin-1-yl)ethyl)thio)-3a,4,5,6,7,7a-hexahydro-1H-benzo[d]imidazol-3-ium chloride
TTC-04—(±)-trans-2-((2-(diethylamino)-2-oxoethyl)thio)-3a,4,5,6,7,7a-hexahydro-1H-benzo[d]imidazol-3-ium chloride
TTC-05—(±)-trans-2-((2-oxo-2-thiomorpholinoethyl)thio)-3a,4,5,6,7,7a-hexahydro-1H-benzo[d]imidazol-3-ium chloride
TTC-06—(±)-trans-2-((2-(4-(methylsulfonyl)piperazin-1-yl)-2-oxoethyl)thio)-3a,4,5,6,7,7a-hexahydro-1H-benzo[d]imidazol-3-ium chloride
TTC-07—(±)-trans-2-((2-oxo-2-(4-phenylpiperazin-1-yl)ethyl)thio)-3a,4,5,6,7,7a-hexahydro-1H-benzo[d]imidazol-3-ium chloride
TTC-08—(±)-trans-2-((2-(4-(tert-butoxycarbonyl)piperazin-1-yl)-2-oxoethyl)thio)-3a,4,5,6,7,7a-hexahydro-1H-benzo[d]imidazol-3-ium chloride
TTC-09—2-((2-morpholino-2-oxoethyl)thio)-4,5-dihydro-1H-imidazol-3-ium chloride
TTC-10—cis-2-((2-oxo-2-(piperidin-1-yl)ethyl)thio)-3a,4,5,6,7,7a-hexahydro-1H-benzo[d]imidazol-3-ium chloride
TTC-11—cis-2-((2-oxo-2-thiomorpholinoethyl)thio)-3a,4,5,6,7,7a-hexahydro-1H-benzo[d]imidazol-3-ium chloride
TTC-12—cis-2-((2-(4-(methylsulfonyl)piperazin-1-yl)-2-oxoethyl)thio)-3a,4,5,6,7,7a-hexahydro-1H-benzo[d]imidazol-3-ium chloride
TTC-13—cis-2-((2-oxo-2-(4-phenylpiperazin-1-yl)ethyl)thio)-3a,4,5,6,7,7a-hexahydro-1H-benzo[d]imidazol-3-ium chloride
TTC-14—(±)-trans-2-((2-(4-methylpiperidin-1-yl)-2-oxoethyl)thio)-3a,4,5,6,7,7a-hexahydro-1H-benzo[d]imidazol-3-ium chloride
TTC-15—(±)-trans-2-((2-(4-hydroxypiperidin-1-yl)-2-oxoethyl)thio)-3a,4,5,6,7,7a-hexahydro-1H-benzo[d]imidazol-3-ium chloride
TTC-16—(±)-trans-2-((2-(4-carboxypiperidin-1-yl)-2-oxoethyl)thio)-3a,4,5,6,7,7a-hexahydro-1H-benzo[d]imidazol-3-ium chloride
TTC-17—(±)-trans-2-((2-oxo-2-(3-oxopiperazin-1-yl)ethyl)thio)-3a,4,5,6,7,7a-hexahydro-1H-benzo[d]imidazol-3-ium chloride
TTC-18—(±)-trans-2-((2-oxo-2-(phenylamino)ethyl)thio)-3a,4,5,6,7,7a-hexahydro-1H-benzo[d]imidazol-3-ium chloride
TTC-19—(+)-(3aR,7aR)-2-((2-morpholino-2-oxoethyl)thio)-3a,4,5,6,7,7a-hexahydro-1H-benzo[d]imidazol-3-ium chloride
TTC-20—(−)-(3aS,7aS)-2-((2-morpholino-2-oxoethyl)thio)-3a,4,5,6,7,7a-hexahydro-1H-benzo[d]imidazol-3-ium chloride
TTC-21—2-((1H-benzo[d]imidazol-2-yl)thio)-1-morpholinoethan-1-one
TTC-22—2-((1H-imidazol-2-yl)thio)-1-morpholinoethan-1-one
TTC-23—1-morpholino-2-((4,5,6,7-tetrahydro-1H-benzo[d]imidazol-2-yl)thio)ethan-1-one
TTC-24—3-(((3aS,7aS)-3a,4,5,6,7,7a-hexahydro-1H-benzo[d]imidazol-2-yl)thio)-1-morpholinopropan-1-one
TTC-25—4-(((3aS,7aS)-3a,4,5,6,7,7a-hexahydro-1H-benzo[d]imidazol-2-yl)thio)-1-morpholinobutan-1-one
TTC-26—(3aR,7aR)-2-((4-methylpiperidin-1-yl)-2-oxoethyl)thio)-3a,4,5,6,7,7a-hexahydro-1H-benzo[d]imidazol-3-ium chloride
TTC-27—(3aS,7aS)-2-((4-methylpiperidin-1-yl)-2-oxoethyl)thio)-3a,4,5,6,7,7a-hexahydro-1H-benzo[d]imidazol-3-ium chloride
TTC-28—(±)-trans-2-((2-(4-ethylpiperidin-1-yl)-2-oxoethyl)thio)-3a,4,5,6,7,7a-hexahydro-1H-benzo[d]imidazol-3-ium chloride
TTC-29—(±)-trans-2-((2-oxo-2-(4-propylpiperidin-1-yl)ethyl)thio)-3a,4,5,6,7,7a-hexahydro-1H-benzo[d]imidazol-3-ium chloride
TTC-30—(±)-trans-2-((2-(4-isopropylpiperidin-1-yl)-2-oxoethyl)thio)-3a,4,5,6,7,7a-hexahydro-1H-benzo[d]imidazol-3-ium chloride
TTC-31—(±)-trans-2-((2-(4-(tert-butyl)piperidin-1-yl)-2-oxoethyl)thio)-3a,4,5,6,7,7a-hexahydro-1H-benzo[d]imidazol-3-ium chloride
TTC-32—(±)-trans-2-((2-oxo-2-(4-(trifluoromethyl)piperidin-1-yl)ethyl)thio)-3a,4,5,6,7,7a-hexahydro-1H-benzo[d]imidazol-3-ium chloride
TTC-33—(±)-trans-2-((2-oxo-2-(4-phenylpiperidin-1-yl)ethyl)thio)-3a,4,5,6,7,7a-hexahydro-1H-benzo[d]imidazol-3-ium chloride
TTC-34—(±)-trans-2-((2-oxo-2-(4-(3-phenylpropyl)piperidin-1-yl)ethyl)thio)-3a,4,5,6,7,7a-hexahydro-1H-benzo[d]imidazol-3-ium chloride
TTC-35—(±)-trans-2-((2-(4-(acetamidomethyl)piperidin-1-yl)-2-oxoethyl)thio)-3a,4,5,6,7,7a-hexahydro-1H-benzo[d]imidazol-3-ium chloride
TTC-36—(±)-trans-2-((2-oxo-2-(4-(o-tolyl)piperazin-1-yl)ethyl)thio)-3a,4,5,6,7,7a-hexahydro-1H-benzo[d]imidazol-3-ium chloride
TTC-37—(±)-trans-2-((2-(4-(4-chlorophenyl)piperazin-1-yl)-2-oxoethyl)thio)-3a,4,5,6,7,7a-hexahydro-1H-benzo[d]imidazol-3-ium chloride
TTC-38—(±)-trans-2-((2-oxo-2-(4-(p-tolyl)piperazin-1-yl)ethyl)thio)-3a,4,5,6,7,7a-hexahydro-1H-benzo[d]imidazol-3-ium chloride
TTC-39—(±)-trans-2-((2-oxo-2-(4-(4-(trifluoromethyl)phenyl)piperazin-1-yl)ethyl)thio)-3a,4,5,6,7,7a-hexahydro-1H-benzo[d]imidazol-3-ium chloride
TTC-40—(±)-trans-2-((-(4-acetylpiperazin-1-yl)-2-oxoethyl)thio)-3a,4,5,6,7,7a-hexahydro-1H-benzo[d]imidazol-3-ium chloride
TTC-41—(±)-trans-2-((2-(4-benzoylpiperazin-1-yl)-2-oxoethyl)thio)-3a,4,5,6,7,7a-hexahydro-1H-benzo[d]imidazol-3-ium chloride

### Hydrates and Solvates

It may be convenient or desirable to prepare, purify, and/or handle a corresponding pharmaceutically acceptable hydrate or solvate of the compound.

The term “solvate” is used herein in the conventional sense to refer to a complex of solute (e.g., compound, salt of compound) and solvent. If the solvent is water, the solvate may be conveniently referred to as a hydrate, for example, a mono-hydrate, a di-hydrate or a tri-hydrate. Unless otherwise specified, a reference to a particular compound also includes hydrate and solvate forms thereof.

### Chemically Protected Forms

It may be convenient or desirable to prepare, purify, and/or handle the compound in a chemically protected form. The term “chemically protected form” is used herein in the conventional chemical sense and pertains to a compound in which one or more reactive functional groups are protected from undesirable chemical reactions under specified conditions (e.g., pH, temperature, radiation, solvent, and the like). In practice, well known chemical methods are employed to reversibly render unreactive a functional group, which otherwise would be reactive, under specified conditions. In a chemically protected form, one or more reactive functional groups are in the form of a protected or protecting group (also known as a masked or masking group, or a blocked or blocking group). By protecting a reactive functional group, reactions involving other unprotected reactive functional groups can be performed, without affecting the protected group; the protecting group may be removed, usually in a subsequent step, without substantially affecting the remainder of the molecule. See, for example, Protective Groups in Organic Synthesis (T. Green and P. Wuts; 4th Edition; John Wiley and Sons, 2006).

A wide variety of such “protecting,” “blocking,” or “masking” methods are widely used and well known in organic synthesis. For example, a compound which has two non-equivalent reactive functional groups, both of which would be reactive under specified conditions, may be derivatized to render one of the functional groups “protected,” and therefore unreactive, under the specified conditions; so protected, the compound may be used as a reactant which has effectively only one reactive functional group. After the desired reaction (involving the other functional group) is complete, the protected group may be “deprotected” to return it to its original functionality.

A hydroxy group may be protected as an ether (—OR) or an ester (—OC(═O)R), for example, as a t-butyl ether; a benzyl, benzhydryl (diphenylmethyl), or trityl (triphenylmethyl) ether; a trimethylsilyl or t-butyldimethylsilyl ether; or an acetyl ester (—OC(═O)CH₃, —OAc).

An amine group may be protected, for example, as an amide (—NRCO—R) or a urethane (—NRCO—OR), for example, as a methyl amide (—NHCO—CH₃); a benzyloxy amide (—NHCO—OCH₂C₆H₅, —NH-Cbz); as a t-butoxy amide (—NHCO—OC(CH₃)₃, —NH-Boc); a 2-biphenyl-2-propoxy amide (—NHCO—OC(CH₃)₂C₆H₄C₆H₅, —NH-Bpoc), as a 9-fluorenylmethoxy amide (—NH—Fmoc), as a 6-nitroveratryloxy amide (—NH—Nvoc), as a 2-trimethylsilylethyloxy amide (—NH—Teoc), as a 2,2,2-trichloroethyloxy amide (—NH-Troc), as an allyloxy amide (—NH—Alloc), or as a 2(-phenylsulfonyl)ethyloxy amide (—NH—Psec).

A prodrug is a form of the compound that, after administration, is metabolized (i.e., converted within the body) into the active pharmaceutical.

### Medical Applications

The compounds of the invention may be employed in the treatment of cancer, neurodegenerative diseases and inflammation.

### Types of Cancer

Cancer is a generic term for a large group of diseases that can affect any part of the body. Other terms used are malignant tumors and neoplasms. One defining feature of cancer is the rapid creation of abnormal cells that grow beyond their usual boundaries, and which can then invade adjoining parts of the body and spread to other organs, the latter process is referred to as metastasizing. Metastases are a major cause of death from cancer.

The cancer may be lung cancer, breast cancer, colorectal cancer, prostate cancer, skin cancer, stomach cancer or liver cancer.

The cancer may be selected from the group consisting of melanoma including ocular, uveal and skin melanoma, head and neck, renal, NSCLC, microsatellite-instable carcinoma including lynch syndrome including gastroesophageal and colorectal, urothelial carcinoma including bladder, merkel cell carcinoma, Hodgkin lymphoma, gastric, oesophageal, non-Hodgkin lymphoma, SCLC, sarkoma, mesothelioma, glioblastoma, microsatellite stable including gastroesophageal and colorectal, pancreas, HCC, prostate, basal cell carcinoma, CTCL, and squamous cell carcinoma.

The cancer may be breast cancer, including estrogen receptor (ER), progesterone receptor (PR), and Her-2 negative breast cancer. In particular, the cancer may be triple negative breast cancer (TNBC), such as metastatic triple negative breast cancer.

The cancer may be multiple myeloma, also known as myeloma, a type of bone marrow cancer. Bone marrow is the spongy tissue at the centre of some bones that produces the body's blood cells. Multiple myeloma often affects several areas of the body, such as the spine, skull, pelvis and ribs.

The cancer may be lung cancer: non-small-cell lung cancer or small-cell lung cancer.

The cancer can be skin cancer, such as basal cell carcinoma (BCC), squamous cell carcinoma (SCC), melanoma and Merkel cell carcinoma (MCC).

In one embodiment the cancer may be selected from breast cancer (e.g. TNBC), multiple myeloma, and lung cancer (e.g. non-small cell lung cancer). The inventors have demonstrated that BAS-2 (1) has benefits in relation to these types of cancer.

The cancer may be a solid tumor.

Use of the compound in the treatment of cancer may comprise use in combination with another cancer treatment such as chemotherapy, radiation therapy or immunotherapy. The chemotherapy may comprise paclitaxel or carboplatin. Use of the compound in the treatment of cancer may comprise use in combination with a metabolic inhibitor.

Use of the compound in the treatment of cancer may comprise identifying the need to regulate glycolytic metabolism.

### Neurodegenerative Diseases

Neurodegenerative diseases are a heterogeneous group of disorders that are characterized by the progressive degeneration of the structure and function of the central nervous system or peripheral nervous system. Common neurodegenerative diseases include Alzheimer's disease and Parkinson's disease.

In one embodiment the neurodegenerative disease is Alzheimer's disease, Huntington's disease, amyotrophic lateral sclerosis (ALS) or epilepsy. In particular, BAS-2 may be employed to treat ALS and/or epilepsy.

### Inflammation

The compound of formula I may be used to modify immune cells. For example, the compound of formula I may be used to modify the metabolism or function of immune cells.

### BRIEF DESCRIPTION OF THE DRAWINGS

FIG. 1: Cell survival of the indicated cell lines following 48 hrs treatment with BAS-2 (n=3, mean±SEM).

FIG. 2: Cell survival of the indicated cell lines following 48 hrs treatment with BAS-2 (n=3, mean±SEM)

FIG. 3: A schematic demonstrating the principle of the GFP-based competition assays. Suppression of genes that alter drug sensitivity leads to changes in the percentage of GFP-positive cells after treatment, which can be used to calculate the RI (left); A heat map showing the response of cells expressing the indicated shRNAs to SAHA and BAS-2. Log-transformed RI values are shown (right).

FIG. 4: Inhibition of trifluoroacetyllysine substrate processing by SAHA (Mean of triplicate measurements) (left); inhibition of trifluoroacetyllysine substrate processing by BAS-2 (Mean of triplicate measurements) (right).

FIG. 5: Representative Western blot showing the levels of acetylated tubulin following treatment with BAS-2 and SAHA with actin as 795 a loading control.

FIG. 6: Western blot of HDAC6 and acetylated tubulin levels in control and HDAC6 K/D cells.

FIG. 7: Proposed modification in the structure of BAS-2 (1).

FIG. 8: Images of 4T1 cells grown in matrigel for 7 days with HDAC6 KO or following treatment with 30 μM BAS-2 (top left) Dot plots show the size of colonies in all treatment groups (n=3, mean±SD) (top right); BALB/cJ mice were subcutaneously inoculated with 4T1 cells and treated with 50 mg/kg BAS-2 for 14 days and tumor weight was plotted (n=10, mean±SEM) (bottom left); MMTv-PyMT tumors were measured and plotted as average total tumor burden following randomization to vehicle (DMSO) or 50 mg/kg BAS-2 (n=4, mean±SEM) (bottom right).

FIG. 9: BAS-2 reduces tumor volume in different cancer models. (A) BALB/cJ mice were subcutaneously inoculated with 4T1 (0.1×10⁶) cells and treated with 50 mg/kg BAS-2 for 14 days. Waterfall plot illustrating response to BAS-2 treatment after 8 days of treatment. Each column represents one mouse, compared to baseline tumor measurement. (B) C57BL/6 mice were subcutaneously inoculated with KP (0.5×10⁶) cells and following tumor formation mice were treated with vehicle or 50 mg/kg BAS-2. The mice were culled at the end of the experiment day 14. Tumors were measured and plotted as percentage change in tumor size following treatment (mean±SEM, n=3, *P-value<0.05, unpaired student T-test). Waterfall plot illustrating response to BAS-2 treatment after 14 days of treatment. Each column represents one mouse, compared to baseline tumor measurement. (C) Weight of mice from 4T1 model taken each week. (D) Weight of mice from KP model taken each week.

FIG. 10 Quantitative proteomics of the BAS-2 acetylome and HDAC6 KD converges on the glycolytic pathway. Volcano plot showing enhanced acetylated peptides following BAS-2 treatment (30 μM) for 48 hrs (left); and for HDAC6 KD compared to control (right); 33 overlapping peptides between BAS-2 treated and HDAC6 KD. The figure in brackets refers to the modification localization score (acetylation) calculated by Maxquant program.

FIG. 11: Chemical inhibition and knockout of HDAC6 reduces glycolysis. (A) Representative ECAR traces (mpH/min/μg protein) are shown (mean±SEM) for MCF10a (n=2) and MDA231 (n=4) cells treated with DMSO or BAS-2 (10 μM) for 24 hrs (B) Fold change in glycolysis and glycolytic capacity (n=2/4, mean±SD). (C and E) Representative ECAR traces for MDA231 cells following HDAC6 KO (C) and treated with BAS-2 (10 μM) for 24 hrs (E). (D and F) Fold change in glycolysis and glycolytic capacity (n=3, mean±SEM). (G) MDA231 cells were traced with 10 mM U¹³C₆ glucose following 10 μM BAS-2 treatment for 24 hrs. (H-K) Percentage of PEP (M+3) (H), 3-PG (M+3) (I), Pyruvate (M+3) (J) and Lactate (K) from glucose are shown (n=3, mean±SEM). (L) Lactate and pyruvate secreted from MDA231 cells treated with 10 μM BAS-2 for 24 hrs (n=3, mean±SEM). (M) Representative ECAR traces for 4T1 cells treated with DMSO or BAS-2 for 24 hrs. (N) Fold change in glycolysis and glycolytic capacity (n=3, mean±SEM). (O) ECAR values (mpH/min/μg protein) shown for 4T1 following HDAC6 KO. (P) Fold change in glycolysis and glycolytic capacity (n=3, mean±SEM).

FIG. 12: Chemical inhibition and knockout of HDAC6 reduces glycolysis (continued) Schematic of in vivo experiment; metabolites extracted from tumors were analysed (n=5, mean±SD). The microenvironment is altered following BAS-2 treatment by reducing pyruvate and lactate in the tumor. Hence this could alter the immune components.

FIG. 13: The effect of inhibition of HDAC6 on metabolic pathways in TNBC and lung cancer models C57BL/6 mice were subcutaneously inoculated with KP cells and following tumor formation mice were treated with vehicle or 50 mg/kg BAS-2. The mice were culled at day 14. Metabolites were extracted from tumors and analyzed by GC-MS (n=4, mean±SEM, unpaired Student T-test). (*P-value<0.05, ***P-value<0.001, 2-way ANOVA with Tukey post hoc test).

FIG. 14: Trans BAS-2 induces cell death and inhibits HDAC6 Trans BAS-2 induces cell death in Multiple Myeloma (MM) cell line JJN3 similar to manufacturer's BAS-2 following 24 hr treatment. However cis-BAS-2 does not induce cell death as measured by annexin v/PI. Analogues 21, 22 and 23 have reduced activity as measured by annexin V/PI.

FIG. 15: TTC-14 induces enhanced cell death and HDAC6 inhibition A series of analogues were tested for induction of cell death. TTC-14 caused greater cell death than Trans-BAS-2 in the JJN3 cells following 24 hr treatment.

FIG. 16: TTC-39 has the lowest IC50 value for HDAC6 inhibition In the next series of analogues TTC-34, TTC37 and TTC-39 induced the most cell death in JJN3 cells following 24 hr treatment. HDAC6 inhibition of TTC-14, TTC-37 and TTC-39 was measured by Reaction Biology Corporation. The IC50 for Trans BAS-2=783 nM, TTC-14=461 nM, TTC-37=756 nM and TTC-39=199 nM.

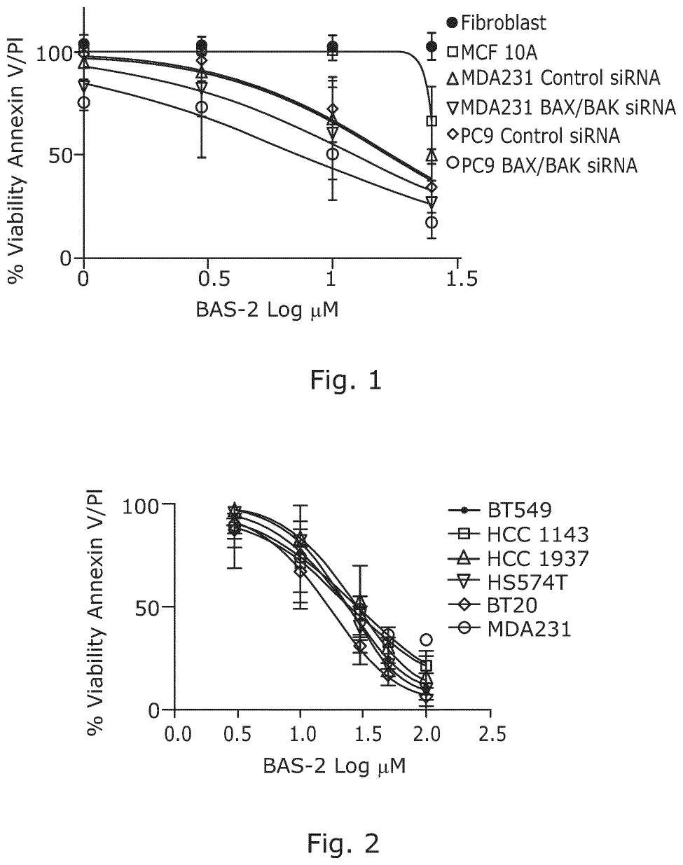

### MATERIALS

### BAS-2 Compound (1)

### 3a,4,5,6,7,7a-hexahydro-1H-benzo[d]imidazole CAS 447410-08-4

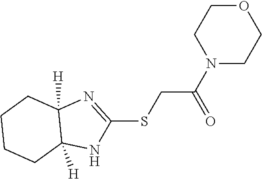 [SMILES: O=C(CSC1=N[C@@H]2CCCC[C@@H]2N1)N1CCOCC1]

BAS-2 (1) was purchased from MCULE (7388843487).

### BAS-2 Analogues

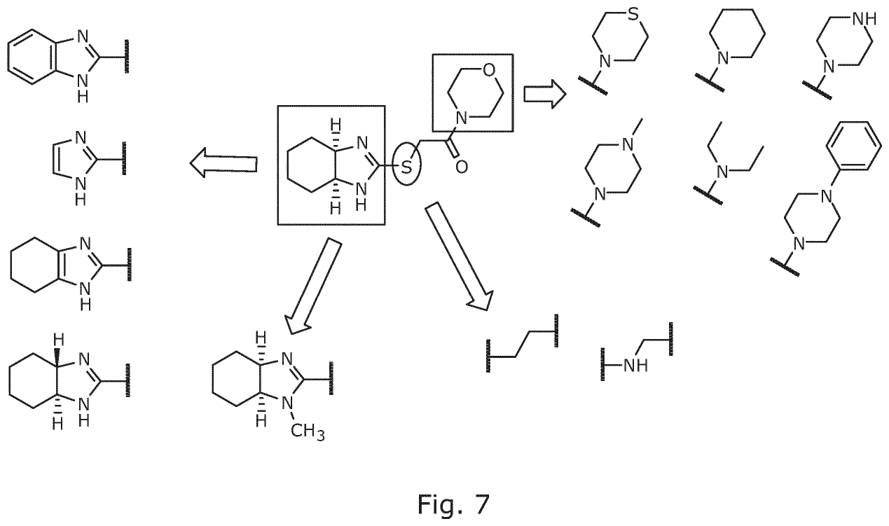

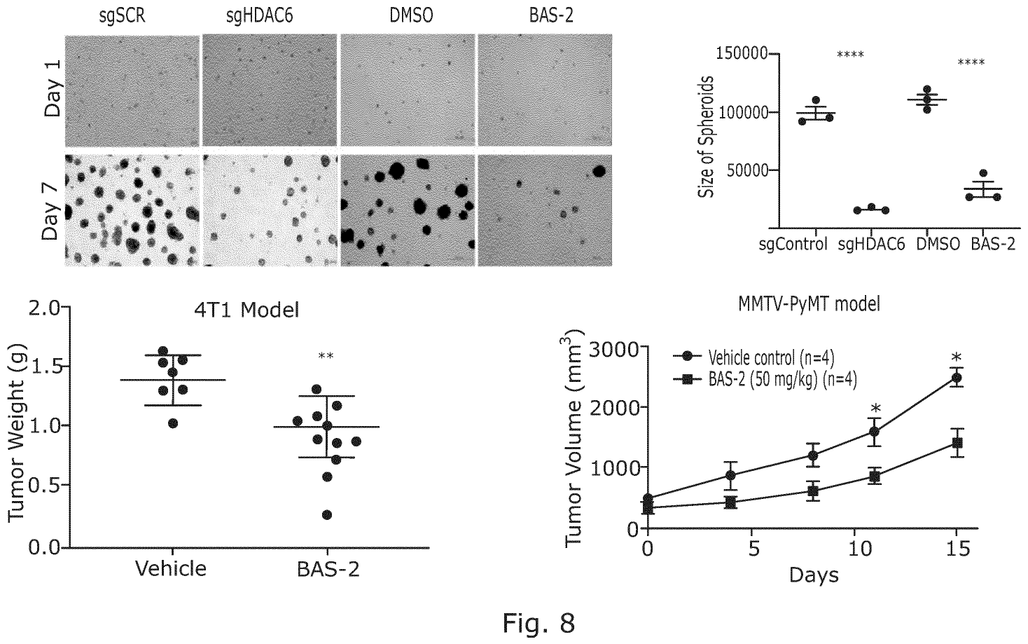

The inventors propose to investigate a series of BAS-2 analogues, examples of which are shown in FIG. 7.

### 1. Synthetic Route for the BAS-2 Analogues

### 1.1. 1H-Benzo[d]Imidazole and 1H-Imidazole Series.

For the synthesis of BAS-2 analogues containing the 1H-benzo[d]imidazole, we could use the methods A and B, described by Mavrova et al. (Mavrova, A.; Anichina, K. K.; Vuchev, D. I.; Tsenov, J. A.; Denkova, P. S.; Kondeva, M. S.; Micheva, M. K., Eur. J. Med. Chem. 2006, 41 (12), 1412-1420.) starting from the 2-mercaptobenzimidazole (4). The same method could be used with the 2-mercaptoimidazole.

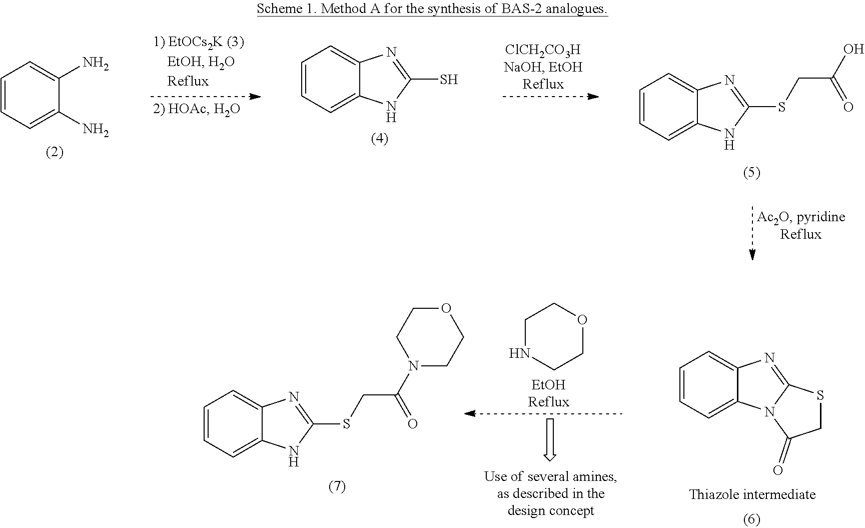 [SMILES: Nc1ccccc1N ; Sc1nc2ccccc2[nH]1 ; O=C(O)CSc1nc2ccccc2[nH]1 ; O=C(CSc1nc2ccccc2[nH]1)N1CCOCC1 ; C1COCCN1 ; O=C1CSc2nc3ccccc3n21]

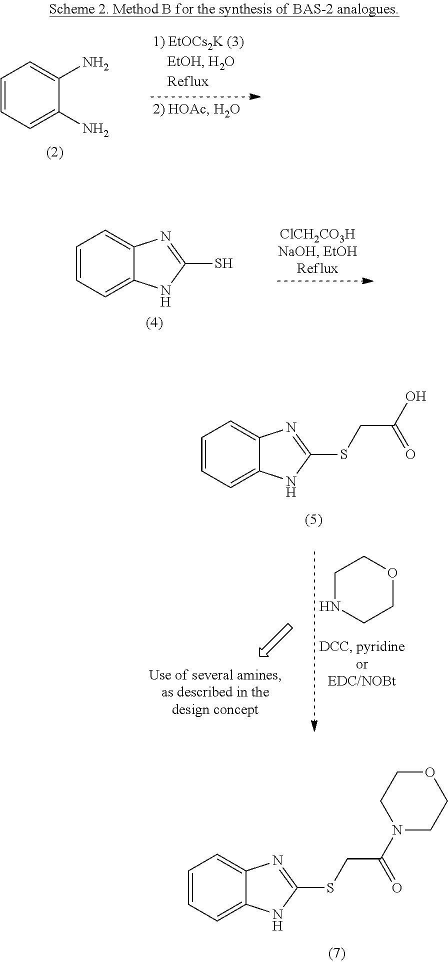 [SMILES: Nc1ccccc1N ; Sc1nc2ccccc2[nH]1 ; O=C(O)CSc1nc2ccccc2[nH]1 ; C1COCCN1 ; O=C(CSc1nc2ccccc2[nH]1)N1CCOCC1]

### 1.2. Trans and Cis Series.

For these two series it will be necessary to synthesize the thiol derivative. We could start from cis-1,2-diaminocyclohexane and (±)-trans-1,2-diaminocyclohexane (racemic), as showed in the Scheme 3 and Scheme 4, respectively.

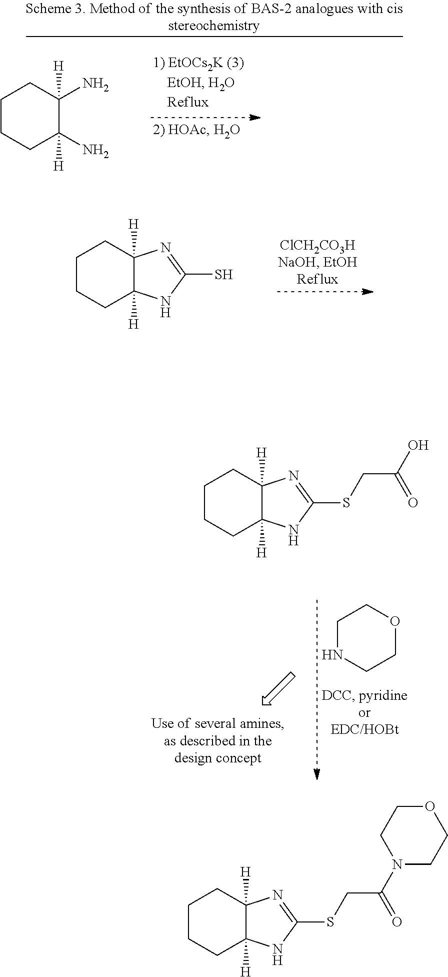 [SMILES: N[C@@H]1CCCC[C@@H]1N ; SC1=N[C@@H]2CCCC[C@@H]2N1 ; O=C(O)CSC1=N[C@@H]2CCCC[C@@H]2N1 ; C1COCCN1 ; O=C(CSC1=N[C@@H]2CCCC[C@@H]2N1)N1CCOCC1]

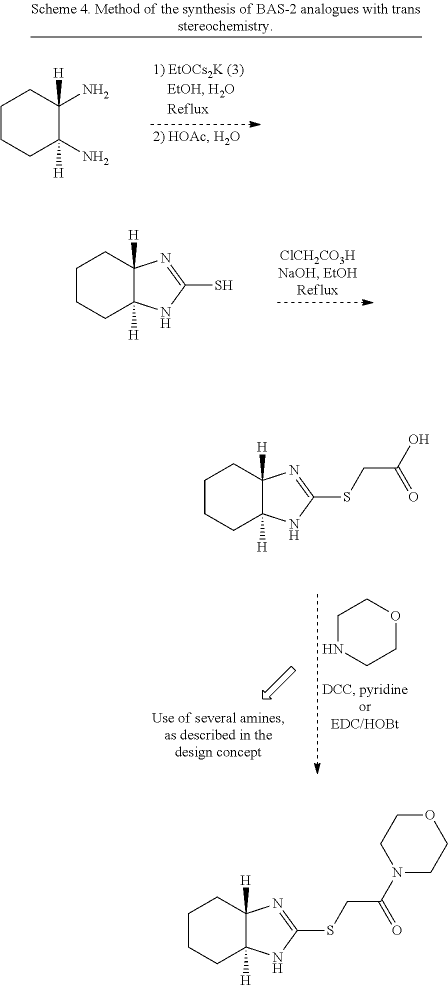 [SMILES: N[C@H]1CCCC[C@@H]1N ; SC1=N[C@H]2CCCC[C@@H]2N1 ; O=C(O)CSC1=N[C@H]2CCCC[C@@H]2N1 ; C1COCCN1 ; O=C(CSC1=N[C@H]2CCCC[C@@H]2N1)N1CCOCC1]

### 1.3. Carbon and Nitrogen Analogues of BAS-2 (1)

For the synthesis of the carbon analogues of BAS-2 (1), we could perform the reaction between the diamine and the succinic anhydride and after perform the cyclization reaction, as described (Scheme 5, Sharma, S et al.; Sharma, S, Gangal, S.; Rauf, A., Eur. J. Med. Chem. 2009, 44 (4), 1751-1757). For the synthesis of the nitrogen analogues of BAS-2 (1), we could use a nucleophilic aromatic substitution reaction after the oxidation of the 2-mercaptobenzimidazole, as described (Scheme 6, Lan et al.: Lan, P.; Romero, F. A.; Malcolm, T. S.; Stevens, B. D.; Wodka, D.; Makara, G. M., Tetrahedron Lett. 2008, 49 (12), 1910-1914.). With this last method, we could try also to incorporate an oxygen series.

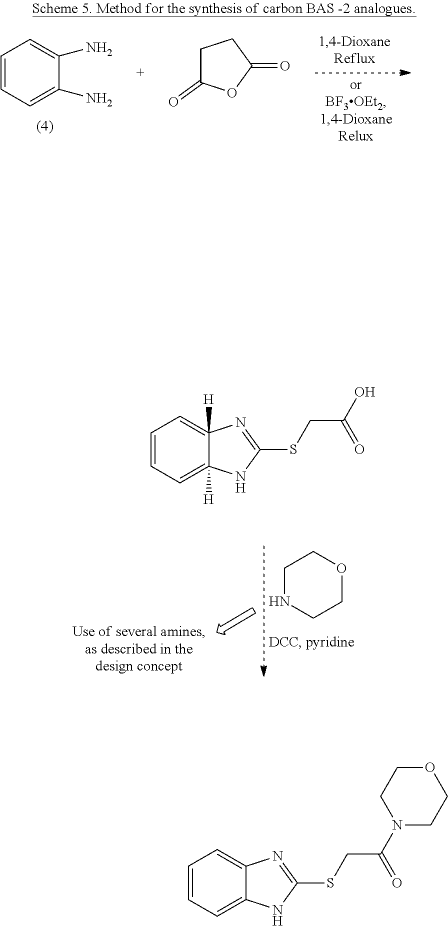

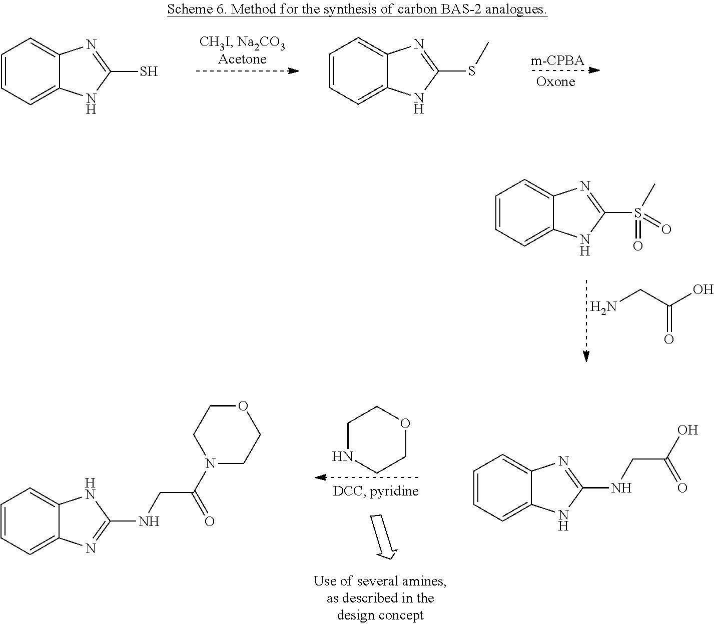

### Results

BAS-2 is a HDAC6 Inhibitor that Impedes TNBC Growth In Vitro and In Vivo

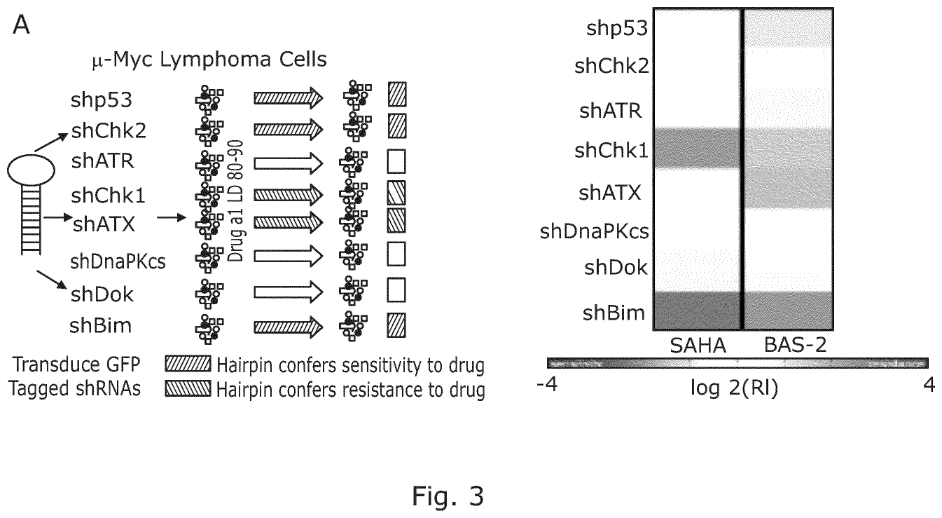

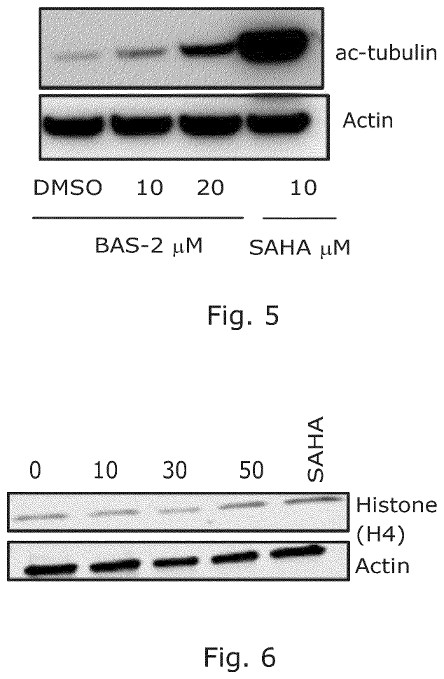

Briefly, Eμ-Myc lymphoma cells are infected with eight different GFP labelled shRNAs and treated with drugs of known mechanism of action or BAS-2. The pattern of resistance and sensitivity is monitored by depletion/enrichment of GFP. By comparing patterns, we can create hypotheses about biochemical modes of action (FIG. 3, left). Following treatment of the infected cells with BAS-2, it emerged that BAS-2 had a novel mechanism of action, which most closely resembled that of Suberoylanilide Hydroxamic Acid (SAHA), a pan histone deacetylase inhibitor (FIG. 3, right, FIG. 5).

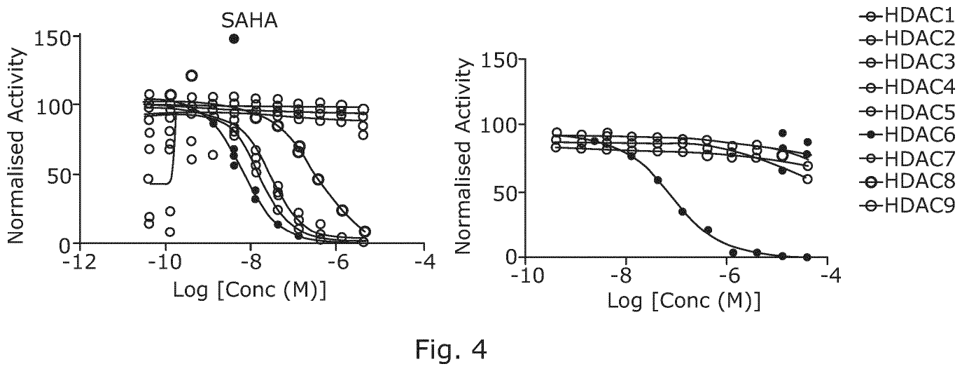

Histone deacetylase (HDAC) are a family of enzymes that modulate their substrates by removing the acetyl group from lysine residues. Using an in vitro HDAC inhibitor screen, we demonstrated that SAHA inhibits the activity of HDAC 1, 2, 6, 7 and 8 (FIG. 4). BAS-2 inhibited only the isozyme HDAC6 with an IC50 of 76 nM (FIG. 4). While there are other highly selective HDAC6 inhibitors, such as Tubacin and CAY10603, BAS-2 only inhibited HDAC6 in the in vitro HDAC assay (>250 fold selectivity). The inventors confirmed an increase in the acetylated tubulin in cells, a known substrate of HDAC6, following treatment with increasing doses of BAS-2 (FIG. 5). BAS-2 treatment did not cause an increase in acetylation of Histone 4 (H4), indicating selective inhibition of HDAC6 in cells. To ensure the compound could induce cell death, we measured the IC50 values by Annexin V/propidium iodide staining across a panel of TNBC cell lines and estrogen receptor positive breast cancer cells lines and found equivalent IC50 values. Phenotypic changes induced by HDAC6 inhibition include prevention of migration and invasion of cancer cells. We detected a significant reduction in both cell migration and invasion following HDAC6 KD in MDA231 cells (FIG. 4), and when treated with BAS-2 both in the MDA231 and BT549 cells (FIGS. 5 and 6). Importantly, there was a reduction in 3D spheroid formation following HDAC6 KD in MDA231 and following treatment with BAS-2 (FIG. 6). Similar results were found in the BT-549 cells following HDAC6 KO and BAS-2 treatment, and in the mouse breast cancer cell line 4T1. No change was found in the proliferation rate of MDA-231 following HDAC6 KD.

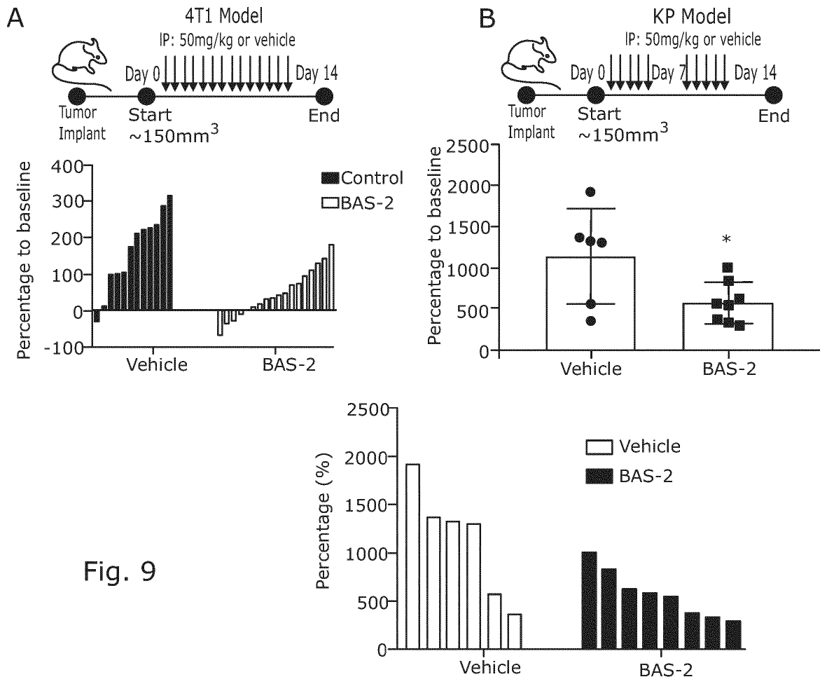

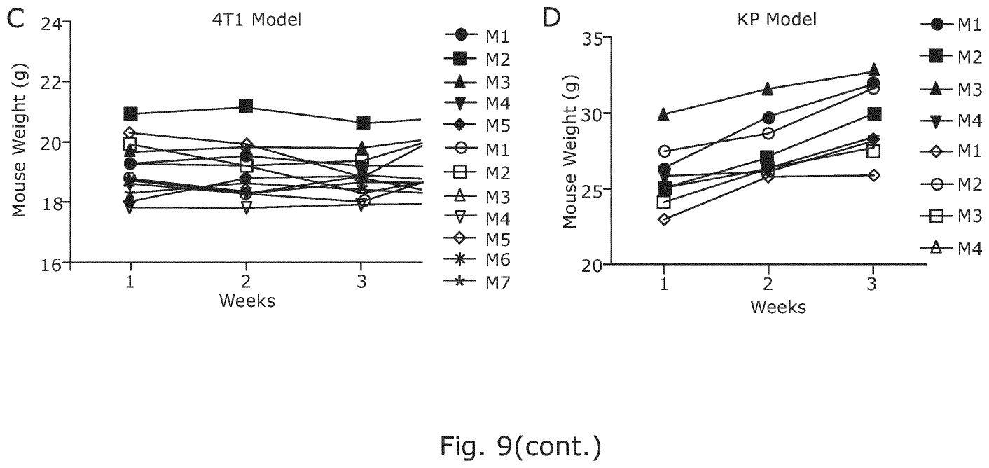

Next, we assessed the in vivo efficacy of BAS-2, as a single agent, using both a syngeneic breast mouse model and a genetic breast mouse model (MMTV-pyMT). Following two-weeks of treatment at 50 mg/kg BAS-2 we found a reduction in tumor volume across the ten tumors per mouse in the aggressive MMTV-pyMT mouse model and a reduction in tumor weight and tumor volume in the 4T1 syngeneic mouse model (FIG. 9). We observed similar effects in the syngeneic Kras^{LSL_G12D} p53^{-/-} (KP) non-small cell lung cancer (NSCLC) mouse mode (FIG. 9) treated over two weeks with 50 mg/kg BAS-2. We did not detect any overt evidence of toxicity, as assessed by weight of the mice while on treatment (FIG. 9).

Combined, using a functional genetic approach we identified BAS-2 as a HDAC6 inhibitor and confirmed similar phenotypic responses between HDAC6 KD or HDAC6 KO and BAS-2 treatment in TNBC cells. In addition, this data suggests that this small molecule, which has not yet been chemically developed, has moderate inhibitory effects as a single agent both in vitro and in vivo.

### Measuring the Acetylome of HDAC6 Using Mass Spectrometry

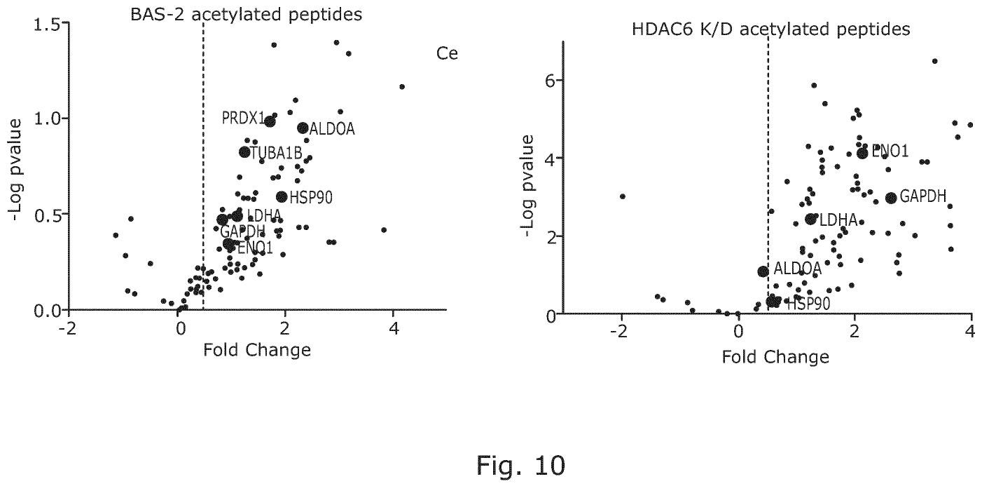

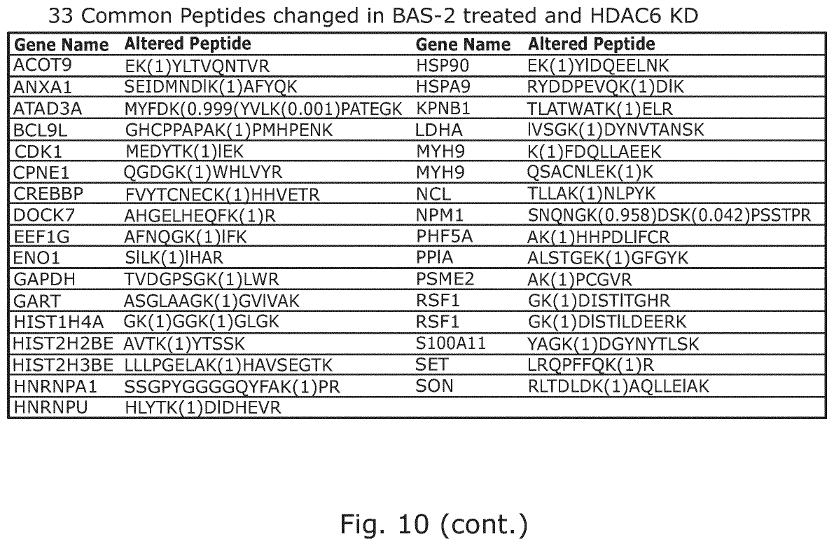

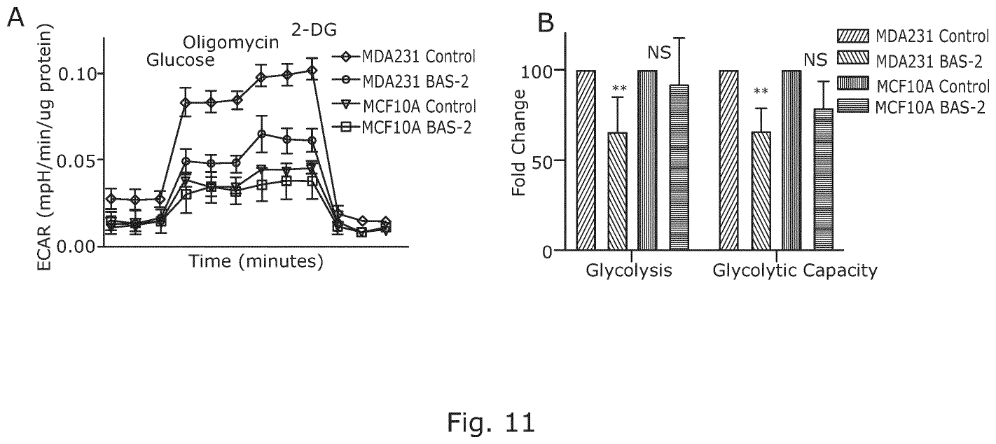

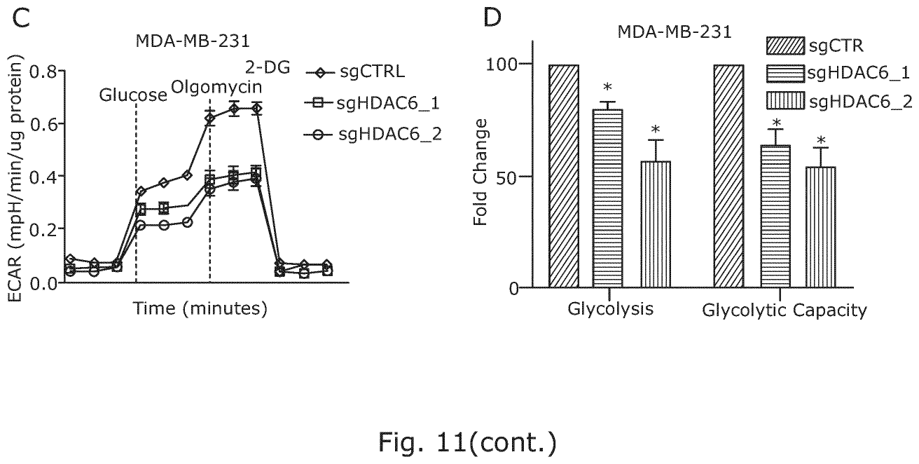

Since we had identified a highly selective HDAC6 inhibitor, we decided to use the compound as a chemical probe to determine HDAC6 substrates in TNBC cells. We utilized advanced mass spectrometry techniques to identify the acetylome of the cells following HDAC6 inhibition, with BAS-2 and compared it to the HDAC6 KD acetylome (23). Since HDAC6 removes acetyl groups from lysine residues, inhibition of HDAC6 should result in an increase in the acetylation levels of its substrates. Following treatment of MDA231 cells with BAS-2 or following HDAC6 KD, we extracted and trypsin digested proteins. The digested peptides were then incubated with anti-acetyllysine to enrich for acetylated peptides. Liquid chromatography-tandem mass spectrometry (LC-MS/MS) was performed on acetylated peptides to identify them and determine relative abundance. LCMS-MaxQuant analysis revealed increased acetylation of 85 proteins in the presence of BAS-2, when compared to the DMSO control. Known substrates of HDAC6 including heat shock protein 90 and tubulin showed increased acetylation by mass spectrometry in the presence of BAS-2, validating our approach. To assess the functional implications of the altered acetylation we conducted pathway analysis on the acetyl-proteome using PANTHER analysis and interestingly found the greatest alteration to be in the metabolic process pathway. This was confirmed using KEGG pathway analysis which demonstrated the acetyl-proteome significantly affected the pathogenic Escherichia coli infection and Glycolysis/Gluconeogenesis pathways (FIG. 10). In the HDAC6 KD cells, we observed a significant increase in the acetylation of a total of 101 proteins compared to the control vector and again we identified some known substrates of HDAC6 (FIG. 10). Interestingly, the functional implications of the altered acetyl-proteome also pointed towards the metabolic process and KEGG pathway enrichment analysis showed significant effect on the Glycolysis/Gluconeogenesis pathway. Notably, we found an increase in the acetylation of 33 of the exact same peptides, from 31 distinct proteins, at the same lysine residue, overlapping between the BAS-2 and HDAC6 KD experiments (shown in FIG. 10).

Similarly, the exact same pathways were altered between the BAS-2 and the HDAC6 KD following PANTHER analysis. This suggests that the novel inhibitor, BAS-2, has a high selectivity for HDAC6 in cells. Excitingly, when mapping the aldolase peptide (DGADFAKWR) the lysine residue (Lysine 201), that had increased acetylation following BAS-2 treatment or HDAC6 KD, was located in the active site of the aldolase protein. Therefore, we measured the activity of aldolase following BAS-2 treatment and found a dose-dependent significant reduction in aldolase enzymatic activity in the MDA231 cells and in the BT-549 cells. We also confirmed a significant reduction in aldolase activity in the HDAC6 KO using CRISPR-Cas9 in the MDA-231 and in the BT-549 cells. Importantly, we confirmed using immunoprecipitation with acetyl beads that HDAC6i with BAS-2 (10 μM 24 hr treatment), or HDAC6 KO increased the acetylation of aldolase. Gyceraldehyde-3-phosphate dehydrogenase (GAPDH) was also acetylated at K192 in both the HDAC6i BAS-2 treated cells and the HDAC6 KD cells. Similarly, we detected a dose dependent reduction in GAPDH activity following HDAC6i with BAS-2 and following HDAC6 KO in MDA-231 (FIG. 30) and BT-549. Along with increased acetylation of GAPDH following BAS-2 treatment and following HDAC6 KO (FIG. 3P). Importantly, protein expression of Aldolase or GAPDH was not altered and did not account for the reduced enzymatic activity. Combined this data shows validation of altered activity and acetylation in two of the glycolytic enzymes identified by mass spectrometry following HDAC6i with BAS-2 and HDAC6 KD.

### The Glycolytic Pathway is Identified in the Interactome of HDAC6

To investigate if substrates which showed increased acetylation following inhibition of HDAC6 or HDAC6 KD actually interacted with HDAC6, we also measured the interactome of HDAC6 using immunoprecipitation and mass spectrometry. HDAC6 was immunoprecipitated from the MDA231 cells and interacting proteins were detected by LC-MS/MS. A total of 332 proteins were identified to significantly interact with HDAC6 compared to IgG control. Known targets such as heat shock protein 90, tubulin and peroxiredoxin-2 were identified. KEGG pathway analysis was used to cluster HDAC6 interacting proteins into biological pathways. HDAC6 significantly interacted with proteins involved in protein production including protein processing, ribosomes and the proteasome pathway. In agreement with acetylome experiments, glycolysis was also identified as a key novel pathway that HDAC6 significantly interacted with. Since this suggested a functional overlap between proteins that had increased acetylation following HDAC6 inhibition, and proteins that physically interacted with HDAC6, we compared both sets of proteins. In total, 15 proteins were statistically significantly altered in common between the three mass spectrometry data sets. Interestingly, when we applied the Cytoscape pathway enrichment analysis to these overlapping proteins we found Glycolysis/Gluconeogenesis to be the only common significantly altered pathway. HDAC6 significantly interacted with GAPDH, enolase and lactate dehydrogenase A (LDHA), along with a large fold change in interaction with aldolase. In the acetyl-proteome experiments HDAC6 inhibition either by BAS-2 or HDAC6 KD caused an increase in the acetylation of aldolase, GAPDH, enolase and LDHA (FIG. 10). Interestingly, using published RNA-sequencing data from TCGA, we found that three of the four glycolytic enzymes are highly expressed in TNBC breast cancer subtypes. In conclusion, using complementary proteomic approaches we have shown for the first time that HDAC6 interacts with key glycolytic enzymes and inhibition of HDAC6 causes an increase in the acetylation of these glycolytic enzymes.

### Inhibition and Knockout of HDAC6 Alters Glycolytic Metabolism in TNBC Cells

Next, we aimed to dissect if the altered acetylation of the glycolytic enzymes leads to a global alteration to glycolytic metabolism following HDAC6 knockout and inhibition of HDAC6 with BAS-2.

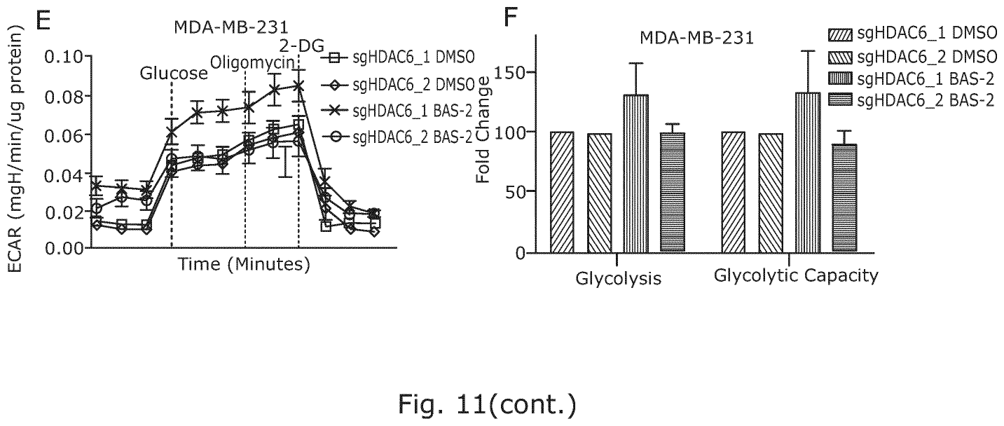

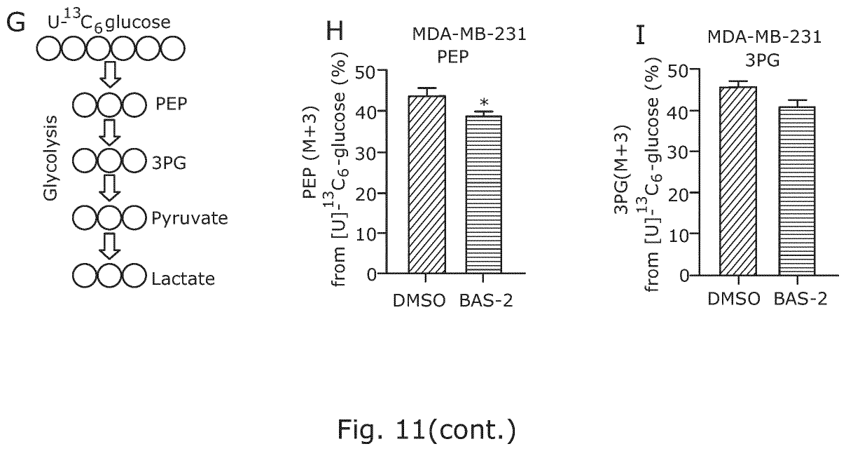

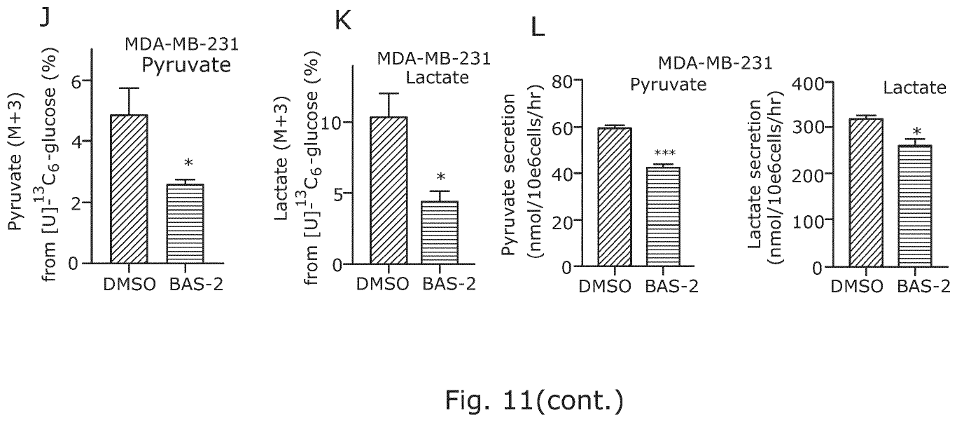

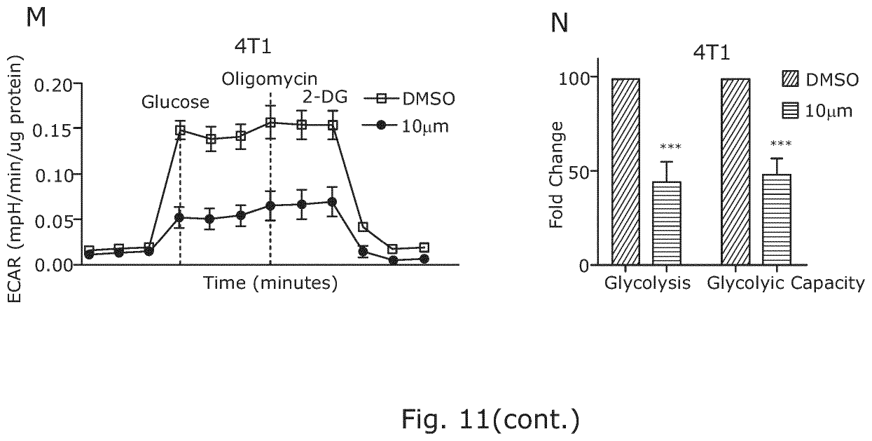

First, we measured the extracellular acidification rate of MDA231 and MCF10a cells following treatment with 10 μM BAS-2 for 24 hr. As is evident, BAS-2 reduced the glycolytic capacity of the MDA231 cells but it did not affect the glycolysis or glycolytic capacity of the non-transformed MCF10a cell line (FIGS. 11A and B). This data suggests a cancer selectivity for the reduction in glycolysis but this would need further investigation. A reduction in ECAR was also observed in the MDA231 treated with the HDAC6 selective inhibitor, Tubacin. Next, we accessed if HDAC6 KO led to a change in the glycolytic capacity of MDA231 cells. Remarkably, following HDAC6 KO there was a significant reduction in ECAR, which is clearly evident with decreased glycolysis and glycolytic capacity in MDA231 cells (FIGS. 11C and D). Importantly, we did not detect any reduction in glycolysis or glycolytic capacity in the HDAC6 KO cells following BAS-2 treatment (FIGS. 11E and F). Highlighting that HDAC6 is required for the BAS-2 induced reduction in glycolysis.

To further confirm the effect of HDAC6i on the glycolytic enzymes, we performed stable isotope tracing and analyzed cell extracts by gas chromatography mass spectrometry (GC-MS). Using U-¹³C₆-glucose, we observed a significant reduction in carbon incorporation from glucose in downstream glycolytic metabolites phosphoenolpyruvate (PEP), 3 phosphoglyceric acid (3PG), lactate and pyruvate following HDAC6i in the MDA231 cells (FIG. 11G-K). We also detected a significant reduction in the secretion of lactate and pyruvate when the MDA231 cells were treated with BAS-2 (FIG. 11L), further suggesting a decrease in glycolysis.

To confirm that the reduction in glycolysis observed with HDAC6 inhibition and knockout was not unique to the MDA231 cell lines we also examined the effects on the 4T1 cell line. We found that both HDAC6 inhibition with BAS-2 and CRISPR-Cas9 knockout of HDAC6 resulted in a decrease in glycolysis and glycolytic flux in the 4T1 cell line (FIG. 11M-P). A similar reduction in ECAR was observed in the 4T1 cells treated with Tubacin. Importantly, we also demonstrated that in the 4T1 HDAC6 KO cells we did not detect any reduction in glycolysis or glycolytic capacity following BAS-2 treatment, again demonstrating that HDAC6 is required for the BAS-2 induced reduction in glycolysis. We confirmed all the above results in the BT549 cell line, with the further addition of ACY-241, another HDAC6 selective inhibitor. Altering glycolysis in the BT-549 cells either through removal of glucose for 24 hr or the addition of galactose, which maintains glycolysis through the LeLoir pathway, increased the cell death induced by BAS-2. This data suggests that BAS-2 induced cell death can be enhanced by altering glycolysis. In summary, in three different cell lines we have confirmed that loss of HDAC6 expression and HDAC6i reduces glycolysis and glycolytic capacity in TNBC cells, by two distinct assays. To ensure that the changes we observed in glycolytic flux were not merely a bi-product of cell death, we assessed cell viability and mitochondrial membrane potential following BAS-2 treatment. We found no loss of mitochondrial membrane potential (FIG. S6A-B) or cell viability (FIG. S6 D-C) at 10 μM BAS-2 treatment, despite initial changes in glycolytic metabolism, suggesting that the reduced glycolytic flux occurs prior to cell death.

Next, we aimed to investigate the effect of HDAC6 inhibition on other metabolic pathways including the pentose phosphate pathway and mitochondrial oxidative respiration. To measure the pentose phosphate pathway, we employed a 1,2-¹³C glucose tracer and analyzed differential label incorporation in downstream glycolytic metabolites lactate and pyruvate. Analysis of label incorporation into these metabolites would suggest a shift towards slightly increased pentose phosphate pathway activity (FIG. S6F). We also measured the oxygen consumption rate (OCR), an indicator of mitochondrial oxidative respiration, following BAS-2 treatment in MDA-231 and BT549 cells and we did not observe any significant changes in OCR or in basal respiration and ATP production at the lower 10 μM dose.

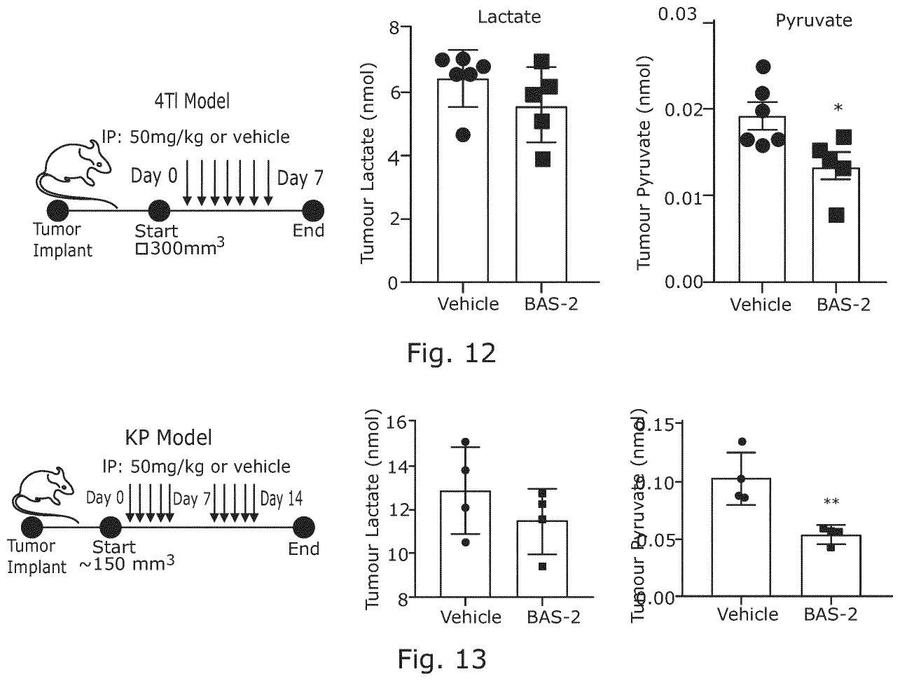

Lastly, to determine whether HDAC6 inhibition with BAS-2 could lead to reduced glycolytic metabolism in vivo, we utilized the 4T1 syngeneic breast cancer model and quantified tumor metabolites following BAS-2 treatment using GC-MS (FIG. 11Q). Our results showed a significant reduction in intratumoral pyruvate levels following BAS-2 treatment (FIG. 12) and a trend towards decreased intratumoral lactate, although this can be difficult to show due to abundance in lactate found in the tumor (FIG. 12). We observed similar results in tumors excised from the syngeneic Kras^{LSL_G12D} p53^{-/-} NSCLC mouse model treated over two weeks with BAS-2 (FIG. 13).

Collectively, these findings confirm, for the first time, a role for HDAC6i in regulating glycolysis both in vitro and in vivo.

### Materials and Methods

### Cell Culture

MDA231, BT-549, HCC1143, HCC1937, HS574T, PC9, 4T1, MCF10a, MDA134, MCF-7, ZR751, TD47 and Fibroblast cells were purchased from American Type Culture Collection (ATCC) and maintained in Roswell Park Memorial Institute medium (RPMI) (Thermo Fisher Scientific, 118757093) or Dulbecco's Modified Eagle's medium (DMEM) (Sigma, D5546) supplemented with 10% (v/v) fetal calf serum (Sigma, F2442), 10 mM L-Glu (Sigma G6392), and 5 mg/ml penicillin/streptomycin (Sigma P4458). For the glucose free experiments glucose-free RPMI (Sigma R1383) was used and glucose (Sigma G7528) or galactose 10 mM (Sigma G5388) added 12 hr before drug treatment. Human mammary epithelial cells were maintained in MEGM mammary epithelial growth media MEGM™ Bullet kit (Lonza CC-3150). The cell lines were recently authenticated by short term tandem repeat profiling in January 2017.

### Western Blotting

Protein samples for Western blot analysis were separated by 12% SDS-PAGE gels. Following separation on the gel, proteins were transferred using electrophoresis onto a nitrocellulose membrane and blocked at room temperature shaking in 5% milk (w/v) in TBS containing 0.5% Tween-20 (TBS-T). Membranes were incubated overnight at 4° C. with primary antibody. HRP tagged secondary antibodies were diluted in TBS-T/5% milk for 1 hr. Antibody reactive bands were detected with LAS-3000, Fujifilm. Fluorescent secondary antibodies were diluted 1:10000 in TBS-T/5% milk for 1 hour. Antibody reactive bands were detected with the Odyssey® infrared imaging system (LI-COR Biosciences). IRDye® 680LT and 800CW-Infrared Dye coupled anti-rabbit or anti-mouse (LI-COR Biosciences).

### Antibodies

Anti-mouse Actin (Sigma, A1978), Ant-rabbit BAX (Cell signalling, 3792S), Anti-rabbit BAK (Cell Signalling, 2774S), Anti-rabbit Acetyl Alpha Tubulin (Cell Signalling, 5335S), Anti-rabbit HDAC6 (Cell Signalling, 7888S), Anti-rabbit Aldolase A (Cell Signalling, 3188S), Anti-rabbit GAPDH (Cell Signalling, 5174S), Anti-Acetyllysine agarose (Immunechem, ICP0388), Anti-Mouse HRP Secondary antibody (LI-COR, 926-80010), Anti-Rabbit HRP Secondary antibody (LI-COR, 926-80011), IRDye® 680LT and 800CW-Infrared Dye coupled anti-rabbit or anti-mouse (LI-COR).

### Generation of HDAC6 Knockout Cell Line

Oligonucleotide sequences for specific short guide RNA (sgRNA) pLentiCRISPRv2 knockout of HDAC6 was designed using the MIT CRISPR oligo algorithm. Two different sgRNA plasmids were developed for two different target sequences within the open reading frame (sgScramble, sgHDAC6-1, sgHDAC6-2). Sequences were located in exon 1 of the open reading frame, and directly followed by a NGG PAM sequences on the 3′ end. Each 20mer oligonucleotide was ligated into the digested lentiviral pLentiCRISPRv2 vector (40). Lentiviral transduction was used to transduce recipient MDA-231, BT549 and 4T1 cells with the respective pLentiCRISPR sgHDAC6 and sgSCR knockout plasmids, and selected for with puromycin (ThermoFisher Scientific, A1113802) to generate stable HDAC6 knockout cell lines.

### Establishment of Stable HDAC6 Knockdown MDA231 Cells.

Scrambled and HDAC6 pLKO shRNA vectors were kindly provided Dr. Hideshima from DFCI. HEK293T cells were transiently transfected with recombinant lentivirus following a standard protocol. The MDA231 were seeded and incubated with 16 hr with culture supernatants from HEK293T cells. The next day the cells were washed with media and selected for antibiotic resistance for one week.

siRNA Transfection

Cells were transiently transfected with siRNA oligonucleotide against BAK (Dharmacon, L-003308-01-0005), BAX (Dharmacon, L-003305-00-0005), or against non-target region (Dharmacon, D-001810-03-05) using LipofectAMINE PLUS (Biosciences, 31985047). Cells were incubated in OPTI-MEM (Biosciences, 13778150) supplemented with 5% FBS for 24 hrs following transfection.

### Apoptosis Assays

Cells were treated with BAS-2 for 24 hrs or 48 hrs where indicated. Apoptosis was assessed by Annexin V/propidium iodide (Biolegend, 640906, Sigma P4170) staining using LSRII Biosciences, Facscanto. Cell viability was detected by CellTiter-Glo® (My Bio, G3582) according to the manufacture's protocol. Mitochondrial membrane potential was measured by adding Tetramethylrhodamine, ethyl ester (TMRE) (Biosciences, T669) at 20 nM for 30 minutes at 37° C. and assessed by flow cytometry with FACS CANTO II.

### Genetic Approach for Drug Target

Epu-Myc p19^{Arf-/-} cells were infected with retroviruses encoding 8 shRNAs (P53, CHK2, CHK1, ATX, DNAPK, BOK, BIM) in pMLS were infected at 10-20% GFP+ proportion as previously described (H. Jiang, J. R. Pritchard, R. T. Williams, D. A. Lauffenburger, M. T. Hemann, A mammalian functional-genetic approach to characterizing cancer therapeutics. Nature chemical biology 7, 92 (2011)). Briefly, individual infected cell populations were seeded at 1 million cells per ml in 48-well plates and treated with drugs. Cells are dosed with equivalent lethal doses rather than equivalent molarities of compound. Cell death was measured by PI exclusion at 48 hours. 80-90% cell death is used to measure GFP enrichment or depletion relative to a vector control at 72 hours. To avoid outgrowth of untreated control cells, the cells were typically seeded at 0.25 million per ml, and 75% of medium was replaced at 24 and 48 hrs. Relative resistance index was calculated as described in Jian et al.

### HDAC Inhibitory Assay

The inhibition of HDAC proteins (HDAC1-9) was determined using a kinetic assay to measure trifluoroacetyllysine substrate processing, as reported previously (J. E. Bradner et al., Chemical phylogenetics of histone deacetylases. Nature chemical biology 6, 238 (2010)).

### Migration and Invasion Assays

Cell migration and invasion experiments were performed using the Boyden Chamber Assay (Corning, CLS3464). 500,000 cells were seeded in serum free RPMI media (Thermo Fisher Scientific, 118757093) in the upper chamber and RPMI media containing 10% FBS (Sigma, F2442), was placed in the lower chamber. For invasion experiments the upper chamber was coated with Matrigel™ (Corning). After 4 hrs cells were treated with BAS-2 (30 μM). Following 24 hrs (migration experiments) or 48 hrs (invasion experiments) treatment cells were stained with 0.5% crystal violet.

### 3-Dimensional Cell Cultures

Individual wells of a 6 well plate was coated with Matrigel™ (Corning) and placed in an incubator at 37° C. for 30 min. 50,000 cells/ml were resuspended in RPMI supplemented with 2% Matrigel™ Cells were placed in Matrigel™ coated wells for 30 min at 37° C., after which RMPI supplemented with 2% Matrigel™ was added to the cultures. Cells were treated with BAS-2 (30 μM) after 48 hrs. Cells were maintained in culture for 7 days in an incubator at 37° C., 5% CO2 and cultures imaged every 24 hrs with Nikon ECLIPSE TE300.

### Mouse Experiments

All experiments involving the MMTV-PyMT animals were reviewed and approved by the Dana-Farber Cancer Institute (DFCI) Institutional Animal Care and Use committee. All experiments involving 4T1 breast mouse model and Kras^{LSL_G12D} p53^{-/-} NSCLC mouse model were reviewed and approved by the Institutional Animal Care and Use Committee (IACUC) at the New York University Langone Health (NYULH). For the transgenic experiments, virgin female FVB/N transgenic mice carrying the polyoma virus middle T-antigen (PyMT) transgene under the control of the mammary tumor virus (MMTV) promoter. The mice had a least one tumor of a size of 300-500 mm³ prior to randomization of treatment of either vehicle (DMSO) or 50 mg/kg BAS-2 IP injection with 5 days on and two days off. For the 4T1 mouse model 1×10⁶ 4T1 cells were implanted into the right and left mammary lower fat pad of BALB/C mice. The mice had a least one tumor of a size of 100-150 mm³ prior to randomization of treatment of either vehicle (DMSO) or 50 mg/kg BAS-2 IP injection every day. Kras^{LSL_G12D} p53^{-/-} (KP) cell lines were established as described previously (F. Li et al., In vivo epigenetic CRISPR screen identifies Asf1a as an immunotherapeutic target in Kras-mutant lung adenocarcinoma. Cancer discovery 10, 270-287 (2020). 5×10⁵ KP cells were subcutaneously injected into both flanks of C57BL/6J mice. The mice had a least one tumor of a size of 100-150 mm³ prior to randomization of treatment of either vehicle (DMSO) or 50 mg/kg BAS-2 IP injection every day.

For all mouse studies, caliper measurements were used to measure tumor volume ((length X width²)/2) and were done in a blinded fashion. The total sum of the tumor burden was calculated by adding the volume of each of the tumors. At the end of treatment, the mice were euthanized humanely in a CO2 chamber.

### Mass Spectrometry-Based Detection of Protein Acetylation

### Preparation of Cell Lysates

MDA231 cells were cultured in the presence of BAS-2 (30 μM) or DMSO for 48 hrs prior to lysis. MDA231 HDAC6 KD and Control Vector cells were seeded 24 hrs prior to the experiment. Cell lysates were prepared in urea lysis buffer (20 mM HEPES pH 8.0, 9 M urea (Sigma, U5378), 1 mM sodium orthovanadate (Sigma, S6508), 2.5 mM sodium pyrophosphate (Sigma, 221368), 1 mM β-glycerophosphate (Sigma, G9422) and sonicated.

### In-Solution Protein Digestion and Reverse-Phase Solid-Phase Extraction of Digests

Sonicated cell lysates were cleared by centrifugation at 20,000×g, and proteins were reduced with Dithiothreitol (DTT) (Sigma, 10197777001) at a final concentration of 1.25 M and alkylated with iodacetamide (Sigma, 16125). For digestion with trypsin, protein extracts were diluted in 20 mM Hepes pH 8.0 to a final concentration of 2 M urea and trypsin was added to digest overnight. Trifluoroacetic acid (TFA) (Bioscience, 28901) was added to protein digest to a final concentration of 1%, precipitate was removed by centrifugation at 2,000 g for 5 min and digests were loaded onto Sep-Pak C-18 columns (Waters, WAT051910) that were equilibrated with 0.1% TFA. Acidified and cleared digest were added prior to washing columns with TFA and acetonitrile (Sigma, 271004). Peptides were eluted with 0.1% TFA in 40% acetonitrile. All peptide fractions were lyophilized.

### Immunoaffinity Purification (IAP) of Acetylated Peptides and LC-MS/MS

Lyophilized peptides were resuspended in IAP buffer (50 mM MOPS pH 7.2, 10 mM sodium phosphate, 50 mM NaCl) and cleared by centrifugation at 10,000×g. Acetylated peptides were enriched using pan-specific anti-acetylated lysine beads (Immunechem, ICPO388). Supernatant was mixed with anti-acetylated lysine beads for 3 h at 4° C. and centrifuged at 2,000×g. Beads were washed and peptides were eluted with 0.15% TFA (Bioscience, 28901). Eluted peptides were concentrated and purified using Stage Tips (Pierce, 87782) and resuspended in 0.1% TFA. Liquid chromatography-tandem mass spectrometry (LC-MS/MS) was performed on the resuspended immunoaffinity purified acetylated peptides.

The samples were analyzed by Systems Biology Ireland (SBI) and Mass Spectrometry Resource (MSR) in University College Dublin on a Thermo Scientific Q Exactive mass spectrometer connected to a Dionex Ultimate 3000 (RSLCnano) chromatography system. Peptides were separated on C18 home-made column (C18RP Reposil-Pur, 100×0.075 mm×1.9 μm) over 150 min at a flow rate of 250 nL/min with a linear gradient of increasing ACN from 1% to 97%. The mass spectrometer was operated in data dependent mode; a high resolution (70,000) MS scan (300-1600 m/z) was performed to select the twelve most intense ions and fragmented using high energy C-trap dissociation for MS/MS analysis.

### Mass Spectrometry Identification of HDAC6 Interactions

MDA231 cells were lysed (50 mM Tris-HCL PH 7.4, 150 mM NACL, 1% Triton x100, 1 mM EDTA and protease inhibitors). Cell lysates were precleared and incubated with anti-HDAC6 antibody (Cell Signalling, 7888S) and A-agarose beads (Pierce, 20333) and incubated at 4° C. overnight. The agarose resin was washed with lysis buffer, and immunoprecipitated proteins were frozen and shipped to TDI Mass Spectrometry Laboratory at the University of Oxford for mass spectrometry analysis. Peptides were analyzed on a Thermo Scientific Q Exactive mass spectrometer connected to a Dionex Ultimate 3000 (RSLCnano) chromatography system. Peptides were loaded in 0.1% trifluroacetic acid (TFA) in 2% ACN onto a trap column (PepMAP C18, 300 μm×5 mm, 5 μm particle, Thermo) and separated on an Easy Spray column (PepMAP C18, 75 μm×500 mm, 2 μm particle, Thermo) with a gradient 2% ACN to 95% ACN in 0.1% formic acid in 5% DMSO. The mass spectrometer was operated in data dependent mode; a high resolution (70,000) MS scan (380 to 1800 m/z) was performed to select the fifteen most intense ions and fragmented using high energy C-trap dissociation for MS/MS analysis.

### Data Processing and Bioinformatics

Raw data from the Q-Exactive was processed using the MaxQuant (version 1.6.2.10) incorporating the Andromeda search engine (42). To identify peptides and proteins, MS/MS spectra were matched against Uniprot Homo sapiens database (2018_04) containing 73,045 entries. All searches were performed using the default setting of MaxQuant, with trypsin as specified enzyme allowing two missed cleavages and a false discovery rate of 1% on the peptide and protein level. The database searches were performed with carbamidomethyl (C) as fixed modification and acetylation (protein N terminus), oxidation (M) and acetylation (K) as variable modifications. For the generation of label free quantitative (LFQ) ion intensities for protein profiles, signals of corresponding peptides in different nano-HPLC MS/MS runs were matched by MaxQuant in a maximum time window of 1 min. The Perseus computational platform (version 1.6.2.3) was used to process MaxQuant results (43). Data was log transformed. For visualization of data using volcano plots, data was log transformed and missing values were imputed with values from a normal distribution, a cut off of t-test difference of 1.5 and a − Log(p-value) of 1.3 were applied. For visualization of acetylated peptides, a t-test difference cut off of 0.5 was applied. In order to evaluate pathway annotation networks of enriched peptides (t-test difference >0.5) from BAS-2 and HDAC6 treated acetylome and significantly enriched proteins (t-test difference >1.5; p-value 0.05 or − Log p-value 1.3) from HDAC6 immunoprecipitation, pathway enrichment analysis was performed using the ClueGo (v2.5.2) (44) and Cluepedia (v1.5.2) (45) plugins in Cytoscape (v3.6.1) (46) with the Homo sapiens (9606) marker set. The KEGG functional pathway databases, consisting of 7425 genes, were used (47). GO tree levels (min=3; max=8) and GO term restriction (min #genes=3, min %=1%) were set and terms were grouped using a Kappa Score Threshold of 0.4. The classification was performed by the two-side hypergeometric statistic test, and its probability value was corrected by the Bonferroni method (Adjusted % Term p-value <0.05).

### Immunoprecipitation

MDA-231 cells were treated with BAS-2 (10 μM) for 24 hrs. Cell lysates were prepared in lysis buffer (50 mM Tris-HCL PH 7.4, 150 mM NACL, 1% Triton x100, 1 mM EDTA and protease inhibitors). Protein was quantified and 500 μg of protein was added to 20 μl of A-agarose beads (Pierce, 20333) and allowed to rotate for 1 hr at 4° C. and 3000 rpm. Proteins were centrifuged for 3 minutes at 3000 rpm at 4° C. Supernatant was added to 500 μl of lysis buffer, 30 pl pan-specific anti-acetylated lysine beads (Immunechem, ICP0388) or control agarose beads, and made up to 1 ml with dH₂O. Samples were rotated overnight at 4° C. at 3000 rpm. Samples were washed with lysis buffer and were resuspended in 30 μl of 2×IP Dye with BME and boiled and ran out on 12% SDS-PAGE gel.

### TCGA Glycolytic Enzyme Analysis

The RNASeq and clinical data was downloaded from TCGA. Male patients, patients with an overall survival time of <=0 days and history of other malignancy were removed. For every gene (plot) a Kruskal-Wallis rank sum test was performed and a Pairwise Wilcoxon rank sum tests between groups if former was statistical significant. P-values of the Pairwise Wilcoxon rank sum test were adjusted for multiple comparison using the Benjamini & Hochberg (1995) method.

### Aldolase and GAPDH Activity Assay

For aldolase and GAPDH activity assay 1×10⁶ MDA231 cells were treated with BAS-2 for 24 hrs prior to experiment. The activity assay was performed as per the manufacturer's instructions (Aldolase: Biovision, K665, GAPDH: Sigma, MAK277). Values were normalized to protein concentrations and fold change was calculated relative to DMSO.

### Extracellular Flux Assay

The bioenergetic function of cells in response to drug treatments was determined using a Seahorse Bioscience XF96 Extracellular Flux Analyzer (Seahorse Bioscience). Cells were seeded in specialized V7 Seahorse tissue culture plates (Agilent, 102601-100) for 24 hrs. Cells were then treated with the indicated concentrations of BAS-2 for a further 24 hrs. One hr prior to experiment, cells were washed and changed to XF Base medium (Agilent, 10353-100) adjusted to pH 7.4. For OXPHOS experiments medium was supplemented with pyruvate (1 mM), L-glutamine (2 mM) and glucose (10 mM). Three baseline oxygen consumption rate (OCR) measurements were taken, followed by three measurements after each injection of oligomycin (1 μM), FCCP (0.5 μM) and Rotenone and Antimycin A (0.5 mM) (Agilent, 103015-100) respectively. For glycolysis experiments medium was supplemented with L-glutamine (1 mM). Three baseline measurements were taken for extracellular acidification rate (ECAR), followed by three measurements after each injection of glucose (10 mM), oligomycin (1 μM) and 2-DG (50 mM) (Agilent, 103020-100) respectively. For all analysis values were normalized to protein concentration before baseline measurements were subtracted. The fold change was calculated relative to DMSO.

### GC-MS Analysis

### Isotope Labelling

Cells were seeded in 12-well tissue culture plates and left to adhere overnight. Cells were then treated with 10 μM of BAS-2 for a further 24 hrs. Following treatment, media was removed and cells were washed with 1×PBS before adding basic RPMI (Thermo Fisher Scientific, 11879020) supplemented with dialyzed FBS (Gibico 26400044) and either 10 mM of [U]-¹³C₆ labelled glucose (Cambridge Isotopes CLM-1396) for 30 minutes or tracing media containing 10 mM 1,2-¹³C labelled glucose (Cambridge Isotopes CLM-504) for 12 h. Cellular metabolites were extracted after a brief wash with 0.9% ice-cold saline solution using a methanol/water/chloroform extraction method, as previously described (48).

### Metabolite Secretion

Media was collected from cells seeded in 6-well plates and cultured in basic RPMI (Thermo Fisher Scientific, 11879020) supplemented with 10% dialyzed FBS (Gibico 26400044) after 24 h treatment with 10 μM of BAS-2. Media was centrifuged at 1,000×g to pellet cell debris and 5 μL of supernatant was extracted in 80% ice-cold methanol mixture containing isotope-labeled internal standards for lactate (Cayman Chemicals CLM-1579), and pyruvate (Cayman Chemicals CLM-2440) and evaporated to dryness.

### Tumor Metabolites

Following 7 or 14 days treatment mice was fasted for 6-8 h prior to euthanasia and tumor harvest. Tumor metabolites were extracted in methanol/water/chloroform using the Qiagen TissueLyser LT, evaporated to dryness and resuspended in 80% methanol. The equivalent volume for 5 mg of tissue was further extracted in 80% ice-cold methanol mixture containing isotope-labeled standards for lactate (Cayman Chemicals CLM-1579), and pyruvate (Cayman Chemicals CLM-2440) and evaporated to dryness.

### Metabolite Derivitization

Metabolite derivitzation using MOX-tBDMS was conducted as previously described (49).

Derivatized samples were analyzed by GC-MS using a DB-35MS column (30 m×0.25 mm i.d.×0.25 μm) installed in an Agilent 7890B gas chromatograph (GC) interfaced with an Agilent 5977B mass spectrometer as previously described (50) and corrected for natural abundance using in-house algorithms adapted from (51).

### BH3 Profiling

The sequence of the BH3-only peptides used and method of synthesis are as previously described (14). BH3 profiling was performed using plate based fluorimetry. Briefly, BH3 peptides (BIM, BIM 0.3 μM, BAD, PUMA, BMF and positive control FCCP) at a 70 μM unless stated otherwise were plated in triplicate on a black 384 well plate. Cells are gently permeabilized with 0.005% digitonin and loaded with the mitochondrial dye 0.5 μM JC-1. The cells are plated on top of the peptides. The loss of mitochondrial potential was measured on the Tecan Saffire² Percentage mitochondrial depolarization, for the peptides is calculated by normalization to the solvent only control DMSO (0%) and the positive control FCCP (100%) using area under the curve.

### Statistical Analysis

Statistical analysis was performed using Graphpad Prism 7 (GraphPad Software, San Diego, CA) unless stated otherwise. The IC₅₀ for the dose response curves following drug treatments was calculated using linear regression curve fit (Log inhibitor v's normalized response). Significance between two groups was determined using an unpaired two-tailed t-test. Significance between more than two groups was determined using a two-way ANOVA with the Tukey post hoc test. For all statistical analysis differences were considered to be statistically significant at * p<0.05 ** P<0.01 *** P<0.001.

### TTC Series

General information. The melting points of the compounds were determined using a Quimis 340 apparatus and are uncorrected. ¹H-NMR spectra were determined in deuterated dimethyl sulfoxide or deuterated chloroform containing approximately 1% tetramethylsilane (TMS) as an internal standard using a Bruker AVANCE 400 at 400 MHz. ¹³C-NMR spectra were resolved using the same spectrometers at 100 MHz and exploited the same solvents. IR spectra (cm-1) were obtained using a Thermo Scientific Nicolet module Smart ITR. The spectroscopic data confirms that the compounds are synthesised.

The progress of all of the reactions was monitored through thin-layer chromatography performed on 2.0×6.0-cm² aluminium sheets precoated with silica gel 60 (HF-254, Merck) to a thickness of 0.25 mm. The developed chromatograms were viewed under ultraviolet light (254-365 nm) and treated with iodine vapor. The reagents and solvents were purchased from commercial suppliers and used as received.

General synthesis of 2H-benzo[d]imidazole-2-thione (Org. Synth., 1950, 30, 56). In a round-bottom flask were added 9.24 mmol of diamine and 1.7 g (11.09 mmol) of potassium ethyl xanthate in 30 mL of ethanol and 1 mL of water. The mixture was heated under reflux for 4 h. After that, a small amount of activated charcoal was added to the reaction and kept under reflux for 30 minutes. The reaction was filtrated and acidified to a pH of 3.5 with a diluted solution of acetic acid in water. The solution was partially concentrated under reduced pressure when it was observed the precipitation of a white solid. It was added more 20 mL of water and the mixture was cooled using an ice bath. The precipitated was filtrated to obtain the pure 2H-benzo[d]imidazole-2-thiones.

### Synthesis of cis-octahydro-2H-benzo[d]imidazole-2-thione

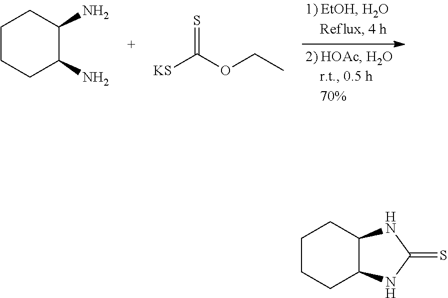

It was obtained 1.02 g of the title compound as glistening yellow crystals (70% yield).

### Synthesis of (±)-trans-octahydro-2H-benzo[d]imidazole-2-thione

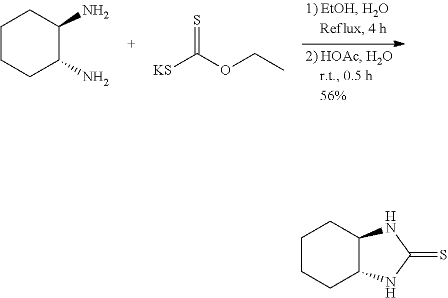

It was obtained 0.817 g of the title compound as glistening white crystals (56% yield).

### Synthesis of (+)-(3aR,7aR)-octahydro-2H-benzo[d]imidazole-2-thione

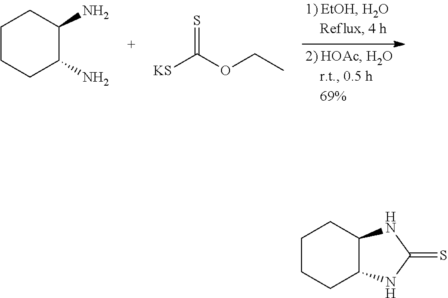

It was obtained 1 g of the title compound as yellowish solid (69% yield). The spectral data is the same of the racemic mixture. α_{D}²⁰=+53.18 (c=1.1; CHCl₃).

### Synthesis of (−)-(3aS,7aS)-octahydro-2H-benzo[d]imidazole-2-thione

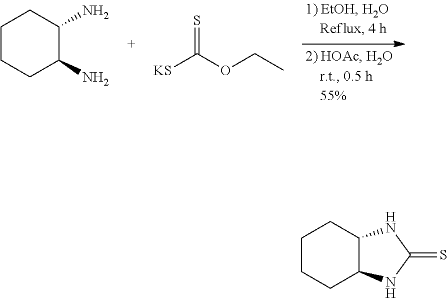

It was obtained 0.791 g of the title compound as yellowish solid (55% yield). The spectral data is the same of the racemic mixture. α_{D}²⁰=−56.18 (c=1.1; CHC13).

### General Methods for the Synthesis of the Chloroacetamides

Method A. In a round-bottom flask were added 22.96 mmol of amine and 4 mL (27.96 mmol) of triethylamine in 60 mL of dichloromethane. The mixture was cooled to 0° C. Subsequently, a solution containing 2 mL (25.25 mmol) of chloroacetyl chloride in 20 mL dichloromethane was slowly added to the solution containing the amine. The reaction mixture was kept under stirring for 30 minutes at 0° C. The reaction was warmed to room temperature and stirred for more 2 h. After that, the mixture was washed with a solution of HCl 1M and after that with a solution of NaHCO₃ saturated. The organic phase was dried over with sodium sulphate and concentrated under reduced pressure. The chloroacetamides were used as obtained from the reaction without further purification.

Method B. In a round-bottom flask were added 1.2 g (9 mmol) of potassium carbonate and 20 mL of water and the solution was stirred under room temperature. After that, 70 mL of THF and 4.5 mmol (or 3.0 mmol) of the amine were added to the solution. The mixture was cooled to 0° C. Subsequently, a solution containing 0.715 mL (9 mmol) of chloroacetyl chloride in 20 mL THF was slowly added to the solution containing the amine. The reaction mixture was kept under stirring for 30 minutes at 0° C. The reaction was warmed to room temperature and stirred for more 2 h. After that, the mixture was concentrated under reduced pressure to remove the THF. 30 mL of water was added to the mixture and it was extracted with AcOEt or filtrated in case of precipitation of the product. The chloroacetamides were used as obtained from the reaction without further purification.

### Synthesis of 2-chloro-1-morpholinoethan-1-one

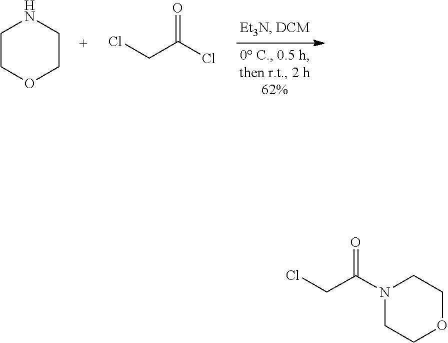 [SMILES: C1COCCN1 ; O=C(Cl)CCl ; O=C(CCl)N1CCOCC1]

It was obtained 2.35 g of the title compound as a brown oil (62% yield).

### Synthesis of 2-chloro-1-(piperidin-1-yl)ethan-1-one

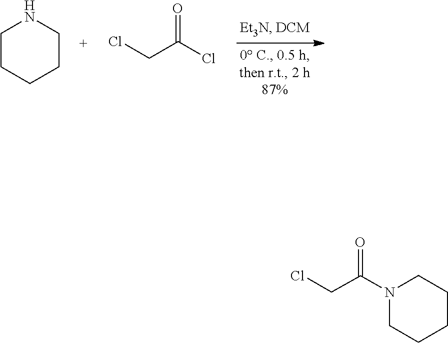 [SMILES: C1CCNCC1 ; O=C(Cl)CCl ; O=C(CCl)N1CCCCC1]

It was obtained 3.23 g of the title compound as a brown oil (87% yield).

### Synthesis of 2-chloro-N,N-diethylacetamide

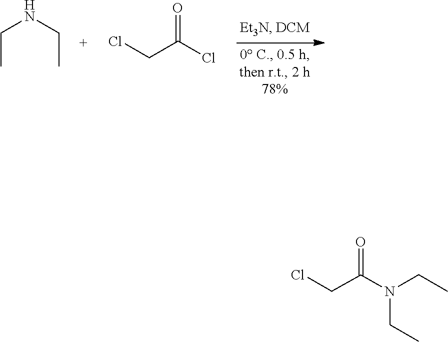

It was obtained 2.68 g of the title compound as a brown oil (78% yield).

### Synthesis of 2-chloro-1-thiomorpholinoethan-1-one

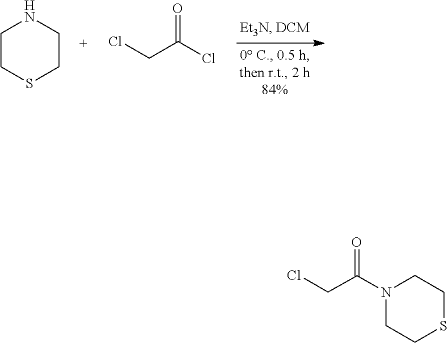 [SMILES: C1CSCCN1 ; O=C(Cl)CCl ; O=C(CCl)N1CCSCC1]

It was obtained 3.5 g of the title compound as a brown oil (84% yield).

### Synthesis of 2-chloro-1-(4-(methylsulfonyl)piperazin-1-yl)ethan-1-one

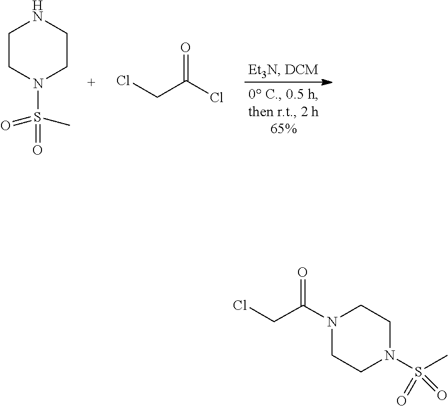

It was obtained 3.6 g of the title compound as a white solid (65% yield).

### Synthesis of 2-chloro-1-(4-phenylpiperazin-1-yl)ethan-1-one

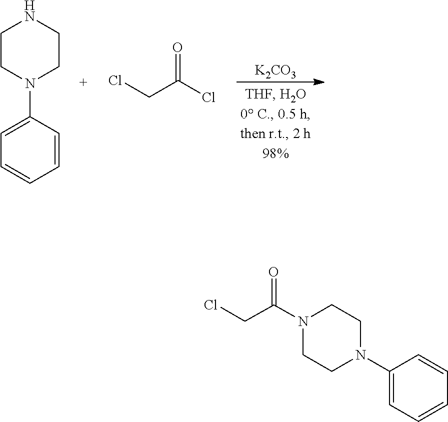

It was obtained 1.05 g of the title compound as white crystals (98% yield).

### Synthesis of tert-butyl 4-(2-chloroacetyl)piperazine-1-carboxylate

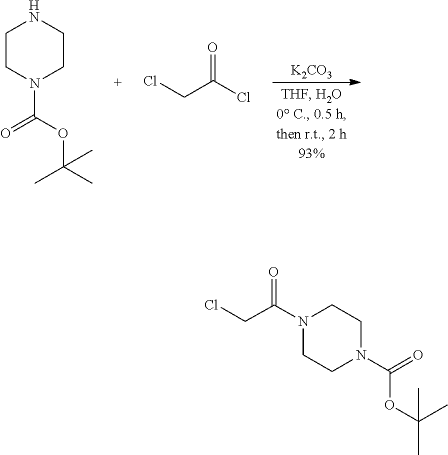

It was obtained 550 mg of the title compound as white crystals (93% yield).

### Synthesis of 2-chloro-1-(4-methylpiperidin-1-yl)ethan-1-one

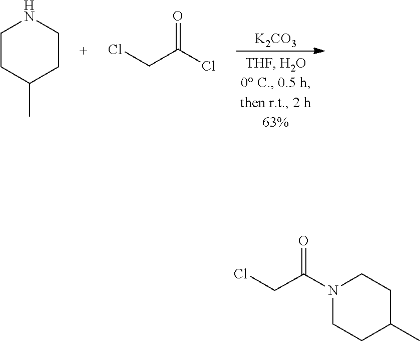

It was obtained 500 mg of the title compound as a colourless oil (63% yield).

### Synthesis of 2-chloro-1-(4-hydroxypiperidin-1-yl)ethan-1-one

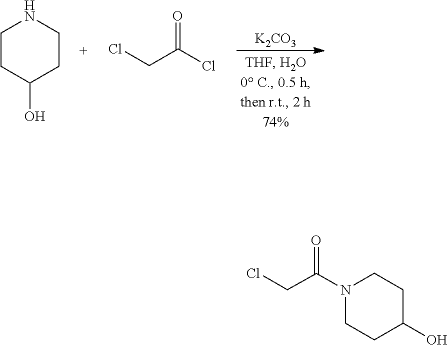

It was obtained 600 mg of the title compound as a colourless oil (74% yield).

### Synthesis of 1-(2-chloroacetyl)piperidine-4-carboxylic acid

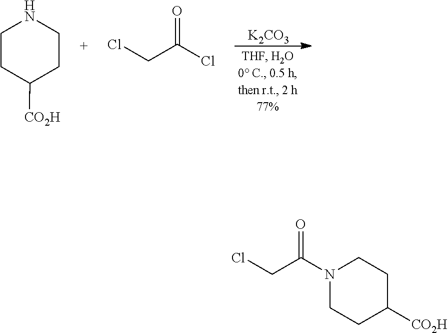

It was obtained 715 mg of the title compound as a white solid (77% yield).

### Synthesis of 4-(2-chloroacetyl)piperazin-2-one

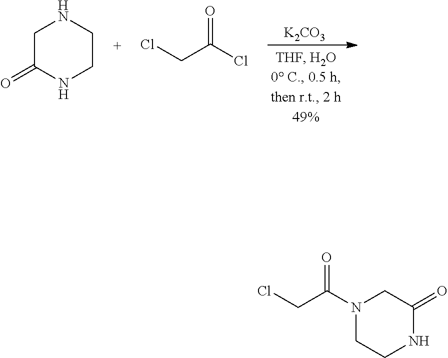

It was obtained 393 mg of the title compound (mixture of conformers) as a white solid (49% yield).

### Synthesis of 2-chloro-N-phenylacetamide

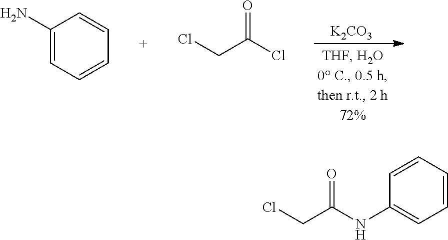

It was obtained 550 mg of the title compound as a white crystals (72% yield).

### Synthesis of 2-chloro-1-(4-ethylpiperidin-1-yl)ethan-1-one

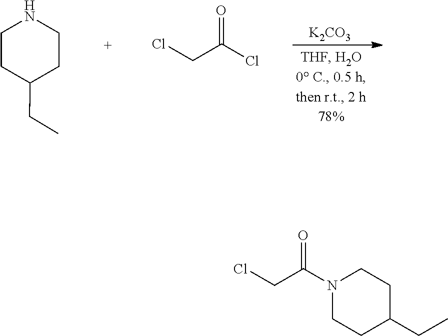

It was obtained 650 mg of the title compound as a colourless oil (78% yield).

### Synthesis of 2-chloro-1-(4-propylpiperidin-1-yl)ethan-1-one

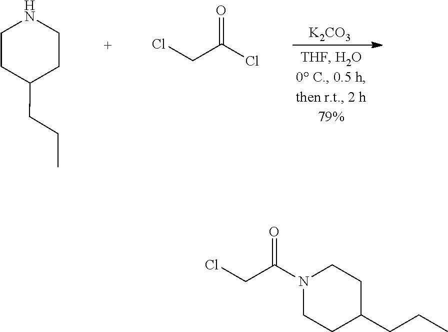

It was obtained 708 mg of the title compound as a colourless oil (79% yield).

### Synthesis of 2-chloro-1-(4-ethylpiperidin-1-yl)ethan-1-one

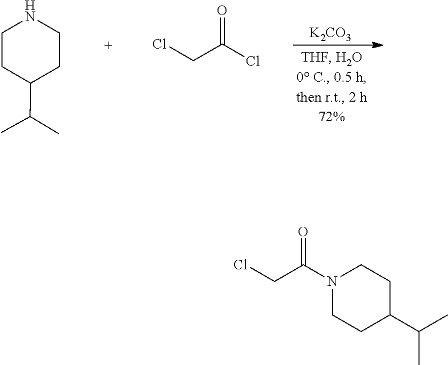

It was obtained 645 mg of the title compound as a colourless oil (78% yield).

### Synthesis of 1-(4-(tert-butyl)piperidin-1-yl)-2-chloroethan-1-one

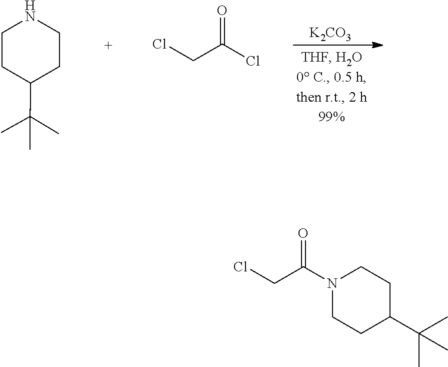

It was obtained 950 mg of the title compound as a white solid (99% yield).

### Synthesis of 2-chloro-1-(4-(trifluoromethyl)piperidin-1-yl)ethan-1-one

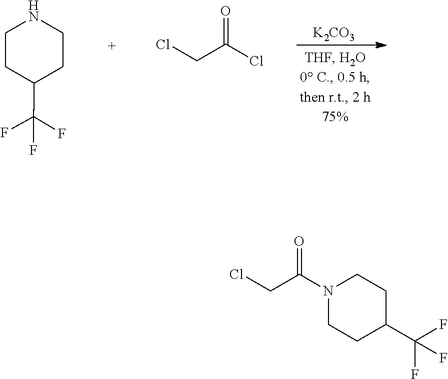

It was obtained 760 mg of the title compound as a colourless oil (75% yield).

### Synthesis of 2-chloro-1-(4-phenylpiperidin-1-yl)ethan-1-one

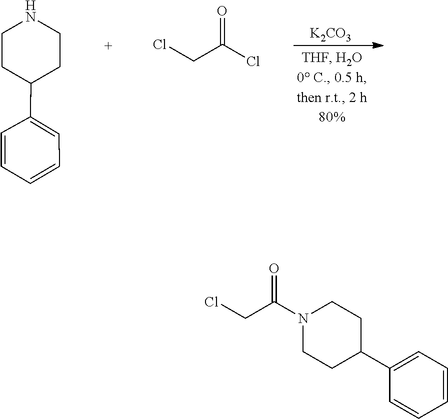

It was obtained 576 mg of the title compound as a white solid (78% yield).

### Synthesis of 2-chloro-1-(4-(3-phenylpropyl)piperidin-1-yl)ethan-1-one

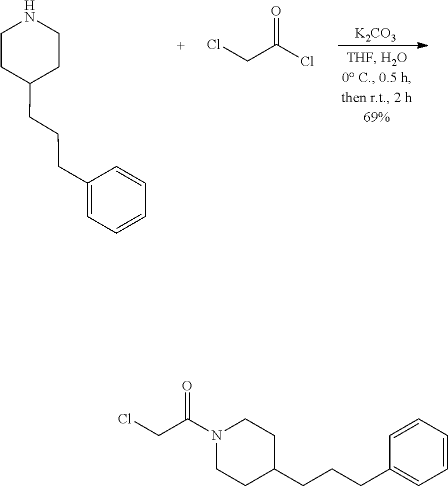

It was obtained 578 mg of the title compound as a colourless oil (69% yield).

### Synthesis of N-((1-(2-chloroacetyl)piperidin-4-yl)methyl)acetamide

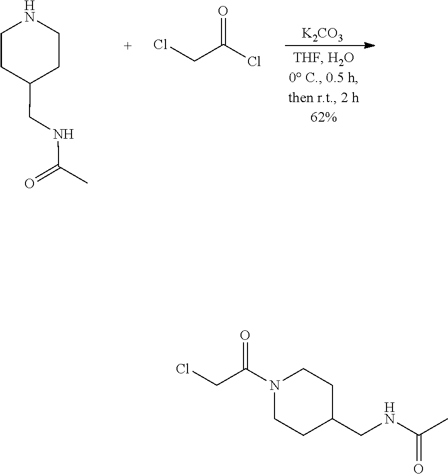

It was obtained 632 mg of the title compound as a colourless oil (62% yield).

### Synthesis of 2-chloro-1-(4-phenylpiperazin-1-yl)ethan-1-one

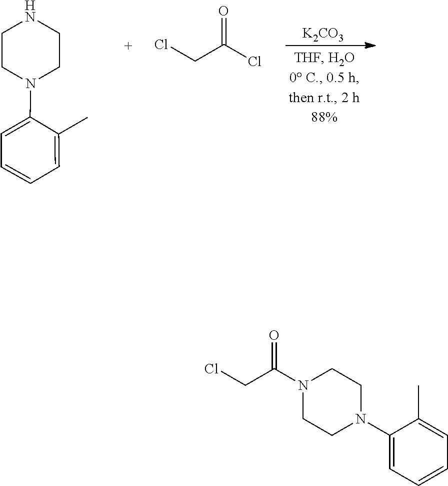

It was obtained 672 mg of the title compound as white solid (88% yield).

### Synthesis of 2-chloro-1-(4-(p-tolyl)piperazin-1-yl)ethan-1-one

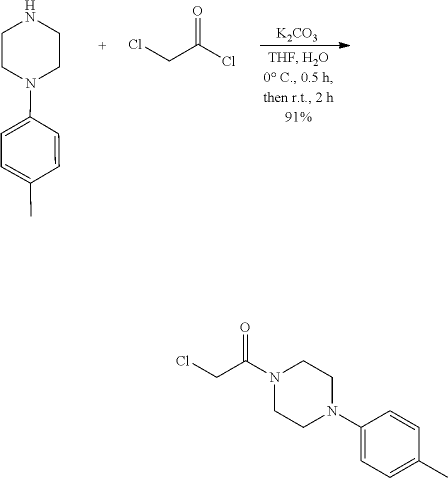

It was obtained 1.02 g of the title compound as white solid (91% yield).

### Synthesis of 2-chloro-1-(4-(p-tolyl)piperazin-1-yl)ethan-1-one

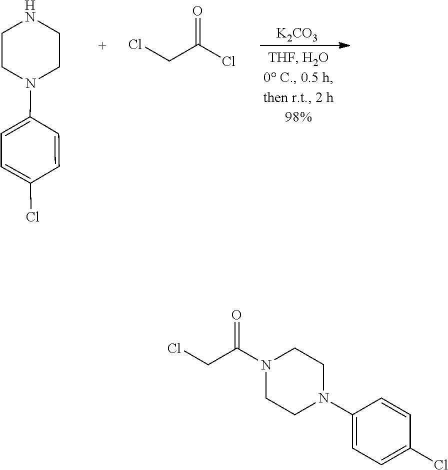

It was obtained 806 mg of the title compound as white solid (98% yield).

### Synthesis of 2-chloro-1-(4-(4-(trifluoromethyl)phenyl)piperazin-1-yl)ethan-1-one

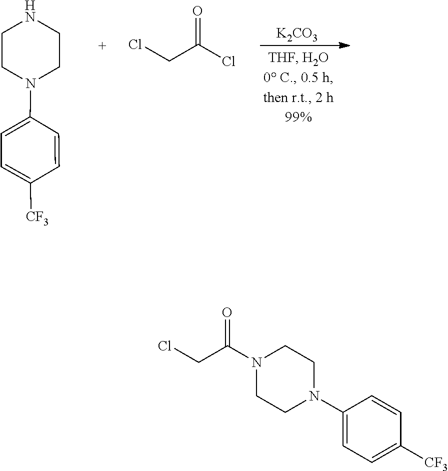

It was obtained 913 mg of the title compound as white solid (99% yield).

### Synthesis of 1-(4-acetylpiperazin-1-yl)-2-chloroethan-1-one

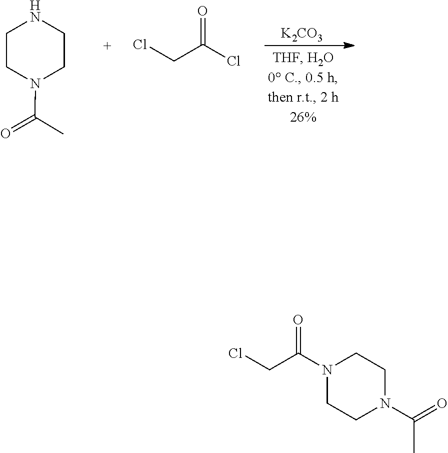

It was obtained 233 mg of the title compound as white solid (26% yield).

### Synthesis of 1-(4-benzoylpiperazin-1-yl)-2-chloroethan-1-one

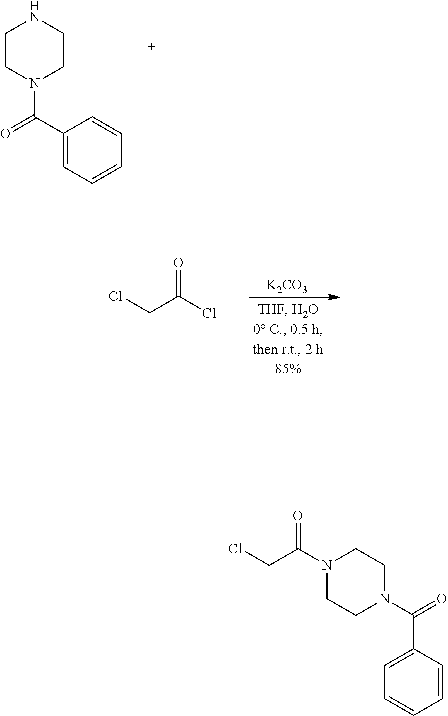

It was obtained 1.1 g of the title compound as colourless oil (85% yield).

General synthesis for the substitution reaction (Org. Biomol. Chem., 2015, 13, 6299). In a round-bottom flask were added 210 mg (1.34 mmol) of (±)-trans-octahydro-2H-benzo[d]imidazole-2-thione or cis-octahydro-2H-benzo[d]imidazole-2-thione and 1.22 mmol of the respective chloroacetamide in 15 mL of acetonitrile. The atmosphere was exchanged for nitrogen. The reaction mixture was kept under stirring at room temperature for 72 hours. At the beginning of the reaction, the solution was homogenous and it was observed the precipitation of a white solid with increasing time. The precipitated was filtrated to obtain the pure products as HCl salts.

### TTC-01 (Trans-BAS-2):

### Synthesis of (±)-trans-2-((2-morpholino-2-oxoethyl)thio)-3a,4,5,6,7,7a-hexahydro-1H-benzo[d]imidazol-3-ium chloride

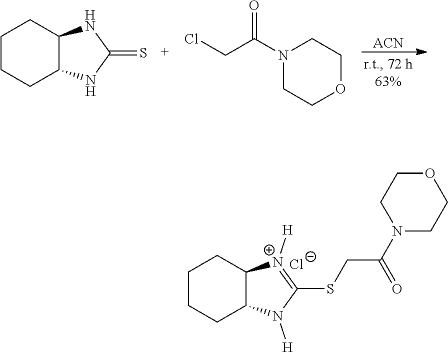

It was obtained 248 mg of the title compound as a white solid (63% yield).

### TTC-02 (Cis-BAS-2):

### Synthesis of cis-2-((2-morpholino-2-oxoethyl)thio)-3a,4,5,6,7,7a-hexahydro-1H-benzo[d]imidazol-3-ium chloride

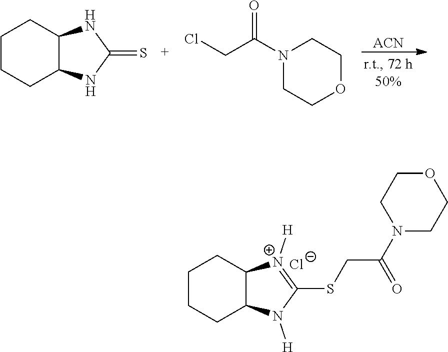

It was obtained 195 mg of the title compound as a white solid (50% yield).

### TTC-03: C Synthesis of (±)-trans-2-((2-oxo-2-(piperidin-1-yl)ethyl)thio)-3a,4,5,6,7,7a-hexahydro-1H-benzo[d]imidazol-3-ium chloride

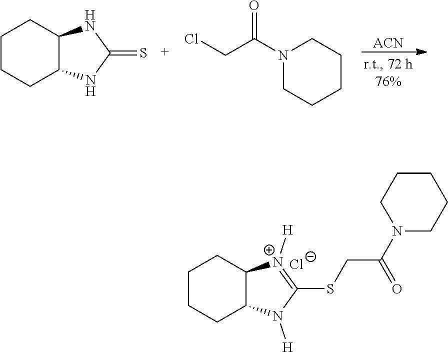

It was obtained 295 mg of the title compound as a white solid (76% yield).

### TTC-04: Synthesis of (±)-trans-2-((2-(diethylamino)-2-oxoethyl)thio)-3a,4,5,6,7,7a-hexahydro-1H-benzo[d]imidazol-3-ium chloride

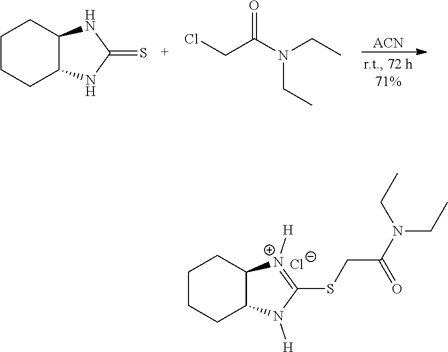

It was obtained 265 mg of the title compound as a white solid (71% yield).

### TTC-05: Synthesis of (±)-trans-2-((2-oxo-2-thiomorpholinoethyl)thio)-3a,4,5,6,7,7a-hexahydro-1H-benzo[d]imidazol-3-ium chloride

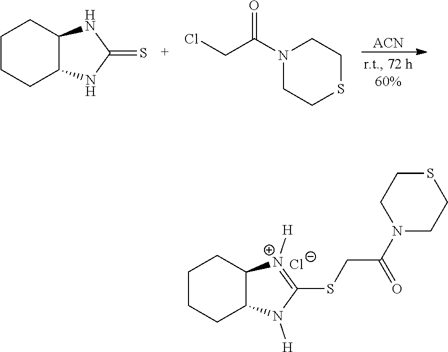

It was obtained 245 mg of the title compound as a white solid (60% yield).

### TTC-06: Synthesis of (±)-trans-2-((2-(4-(methylsulfonyl)piperazin-1-yl)-2-oxoethyl)thio)-3a,4,5,6,7,7a-hexahydro-1H-benzo[d]imidazol-3-ium chloride

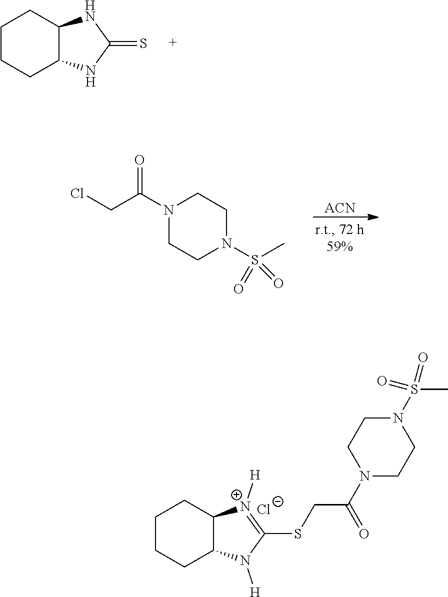

It was obtained 288 mg of the title compound as a white solid (59% yield).

### TTC-07: Synthesis of (±)-trans-2-((2-oxo-2-(4-phenylpiperazin-1-yl)ethyl)thio)-3a,4,5,6,7,7a-hexahydro-1H-benzo[d]imidazol-3-ium chloride

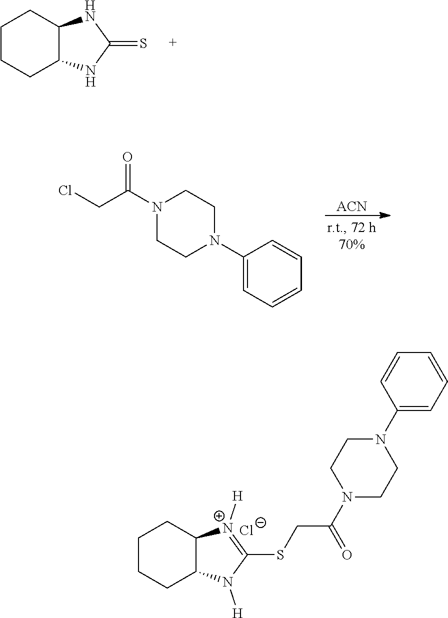 [SMILES: S=C1N[C@@H]2CCCC[C@H]2N1 ; O=C(CCl)N1CCN(c2ccccc2)CC1 ; "ACN" ; O=C(CSC1=[NH+][C@@H]2CCCC[C@H]2N1)N1CCN(c2ccccc2)CC1 ; [Cl-]]

It was obtained 337 mg of the title compound as a white solid (70% yield).

### TTC-08: Synthesis of (±)-trans-2-((2-(4-(tert-butoxycarbonyl)piperazin-1-yl)-2-oxoethyl)thio)-3a,4,5,6,7,7a-hexahydro-1H-benzo[d]imidazol-3-ium chloride

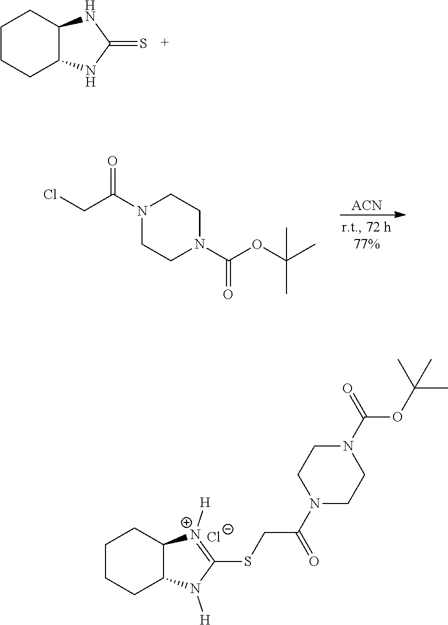

It was obtained 393 mg of the title compound as a white solid (77% yield).

TTC-09: Synthesis of 2-((2-morpholino-2-oxoethyl)thio)-4,5-dihydro-1H-imidazol-3-ium chloride (Org. Biomol. Chem., 2015, 13, 6299). In a round-bottom flask were added 137 mg (1.34 mmol) of imidazolidine-2-thione and 1.22 mmol of 2-chloro-1-morpholinoethan-1-one in 15 mL of acetonitrile. The atmosphere was exchanged for nitrogen. The reaction mixture was kept under stirring at room temperature for 72 hours. At the beginning of the reaction, the solution was homogenous and it was observed the precipitation of a white solid with increasing time. The precipitated was filtrated to obtain the pure product as HCl salt.

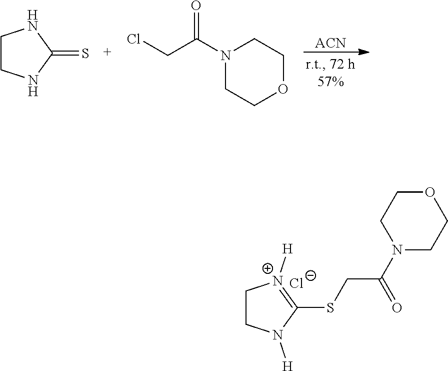

It was obtained 186 mg of the title compound as a white solid (57% yield).

### TTC-10: Synthesis of cis-2-((2-oxo-2-(piperidin-1-yl)ethyl)thio)-3a,4,5,6,7,7a-hexahydro-1H-benzo[d]imidazol-3-ium chloride

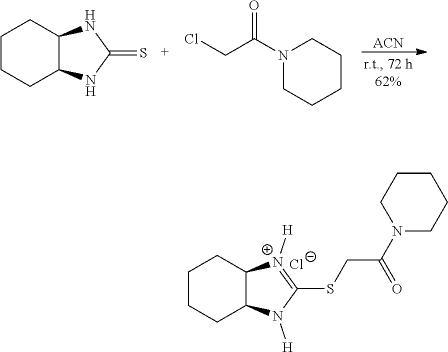

It was obtained 241 mg of the title compound as a white solid (62% yield).

### TTC-11: Synthesis of cis-2-((2-oxo-2-thiomorpholinoethyl)thio)-3a,4,5,6,7,7a-hexahydro-1H-benzo[d]imidazol-3-ium chloride

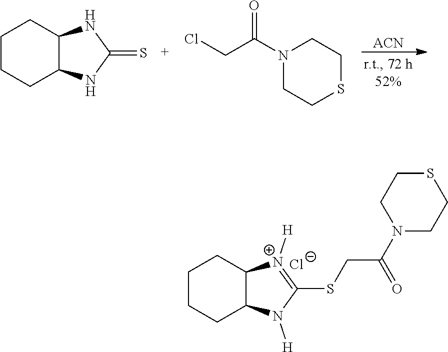

It was obtained 216 mg of the title compound as a white solid (52% yield).

### TTC-12: Synthesis of cis-2-((2-(4-(methylsulfonyl)piperazin-1-yl)-2-oxoethyl)thio)-3a,4,5,6,7,7a-hexahydro-1H-benzo[d]imidazol-3-ium chloride

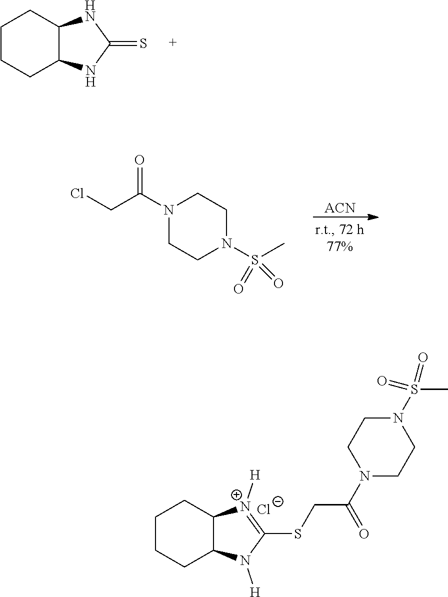

It was obtained 376 mg of the title compound as a white solid (77% yield).

### TTC-13: Synthesis of cis-2-((2-oxo-2-(4-phenylpiperazin-1-yl)ethyl)thio)-3a,4,5,6,7,7a-hexahydro-1H-benzo[d]imidazol-3-ium chloride

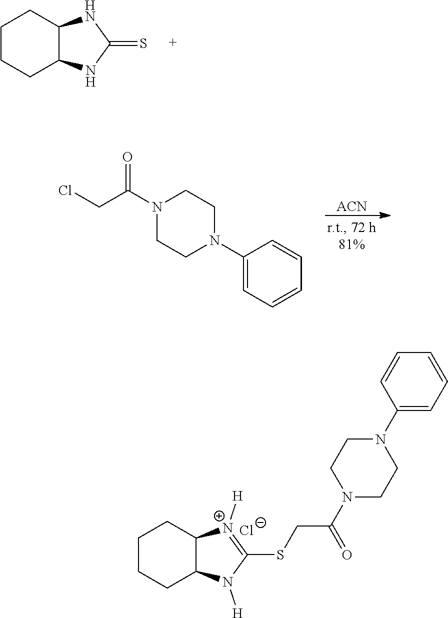 [SMILES: S=C1N[C@H]2CCCC[C@H]2N1 ; O=C(CCl)N1CCN(c2ccccc2)CC1 ; "ACN" ; O=C(CSC1=[NH+][C@@H]2CCCC[C@@H]2N1)N1CCN(c2ccccc2)CC1 ; [Cl-]]

It was obtained 390 mg of the title compound as a white solid (81% yield).

### TTC-14: Synthesis of (±)-trans-2-((2-(4-methylpiperidin-1-yl)-2-oxoethyl)thio)-3a,4,5,6,7,7a-hexahydro-1H-benzo[d]imidazol-3-ium chloride

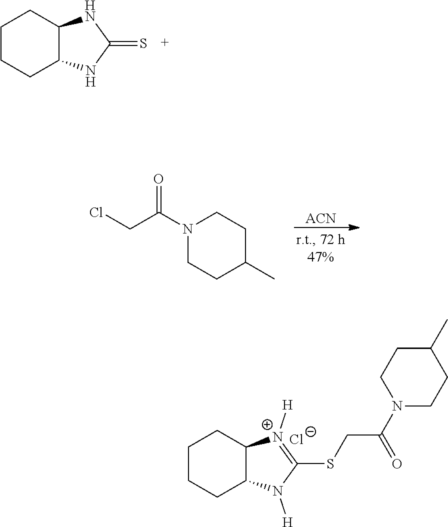

It was obtained 191 mg of the title compound (mixture of conformers) as a white solid (47% yield).

### TTC-15: Synthesis of (±)-trans-2-((2-(4-hydroxypiperidin-1-yl)-2-oxoethyl)thio)-3a,4,5,6,7,7a-hexahydro-1H-benzo[d]imidazol-3-ium chloride

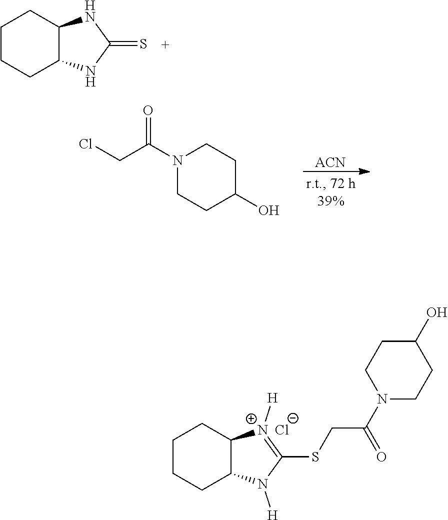 [SMILES: S=C1N[C@@H]2CCCC[C@H]2N1 ; O=C(CCl)N1CCC(O)CC1 ; "ACN" ; O=C(CSC1=[NH+][C@@H]2CCCC[C@H]2N1)N1CCC(O)CC1 ; [Cl-]]

It was obtained 161 mg of the title compound (mixture of conformers) as a white solid (39% yield).

### TTC-16: Synthesis of (±)-trans-2-((2-(4-carboxypiperidin-1-yl)-2-oxoethyl)thio)-3a,4,5,6,7,7a-hexahydro-1H-benzo[d]imidazol-3-ium chloride

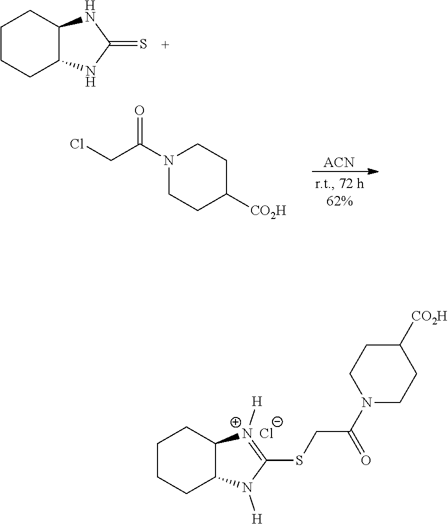 [SMILES: S=C1N[C@@H]2CCCC[C@H]2N1 ; O=C(O)C1CCN(C(=O)CCl)CC1 ; "ACN" ; O=C(O)C1CCN(C(=O)CSC2=[NH+][C@@H]3CCCC[C@H]3N2)CC1 ; [Cl-]]

It was obtained 250 mg of the title compound (mixture of conformers) as a white solid (62% yield).

### TTC-17: Synthesis of (±)-trans-2-((2-oxo-2-(3-oxopiperazin-1-yl)ethyl)thio)-3a,4,5,6,7,7a-hexahydro-1H-benzo[d]imidazol-3-ium chloride

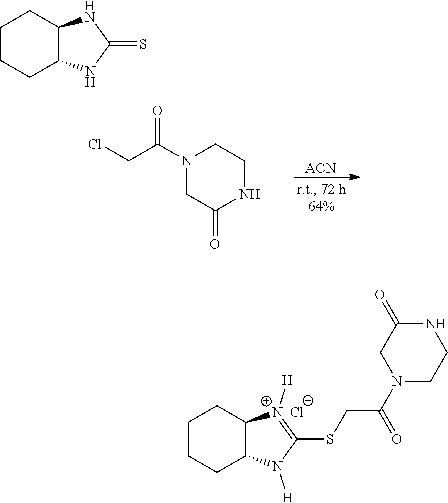

It was obtained 263 mg of the title compound (mixture of conformers) as a white solid (64% yield).

### TTC-18: Synthesis of (±)-trans-2-((2-oxo-2-(phenylamino)ethyl)thio)-3a,4,5,6,7,7a-hexahydro-1H-benzo[d]imidazol-3-ium chloride

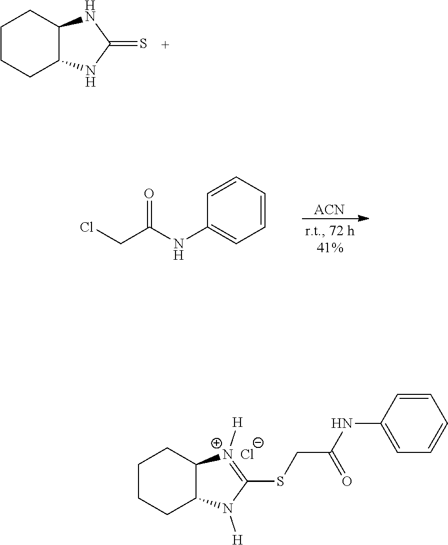

It was obtained 164 mg of the title compound as a white solid (41% yield).

### TTC-19 (R,R): Synthesis of (+)-(3aR,7aR)-2-((2-morpholino-2-oxoethyl)thio)-3a,4,5,6,7,7a-hexahydro-1H-benzo[d]imidazol-3-ium chloride

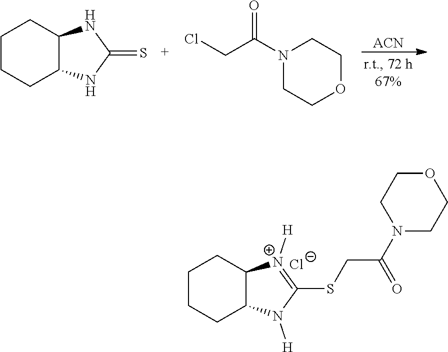

It was obtained 264 mg of the title compound as a white solid (67% yield). The spectral data is the same of the racemic mixture. α_{D}²⁰=+47.5 (c=1.0; H₂O).

### TTC-20 (S,S): Synthesis of (−)-(3aS,7aS)-2-((2-morpholino-2-oxoethyl)thio)-3a,4,5,6,7,7a-hexahydro-1H-benzo[d]imidazol-3-ium chloride

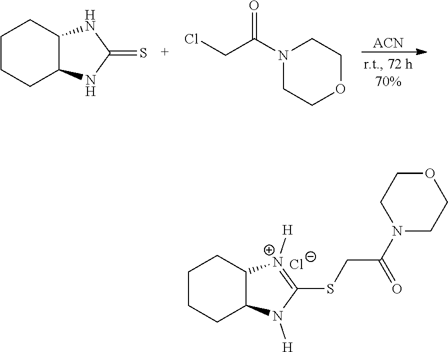

It was obtained 273 mg of the title compound as a white solid (70% yield). The spectral data is the same of the racemic mixture. α_{D}²⁰=−42.5 (c=1.0; H₂O).

General Procedure for the synthesis of TTC-21, TTC-22 and TTC-23: In a round-bottom flask were added 1.296 mmol) of the imidazole-2-thiol derivative, 202 mg (1.23 mmol) of 2-chloro-1-morpholinoethan-1-one and 204 mg (1.476 mmol) of potassium carbonate in 15 mL of acetone. The reaction mixture was kept under stirring at room temperature for 18 hours. The solvent was removed under reduced pressure and 25 mL of water was added. The mixture was extracted with AcOEt. The organic phase was dried over with Na₂SO4 and removed under reduced pressure. The crude was purified using column chromatography, using first AcOEt as eluent to remove apolar impurities and the product was recovered using 5 to 10% of MeOH in DCM.

### TTC-21: Synthesis of 2-((1H-benzo[d]imidazol-2-yl)thio)-1-morpholinoethan-1-one

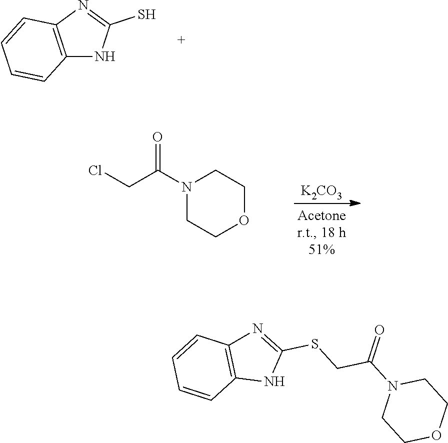

It was obtained 178 mg of the title compound as a white solid (51% yield).

### TTC-22: Synthesis of 2-((1H-imidazol-2-yl)thio)-1-morpholinoethan-1-one

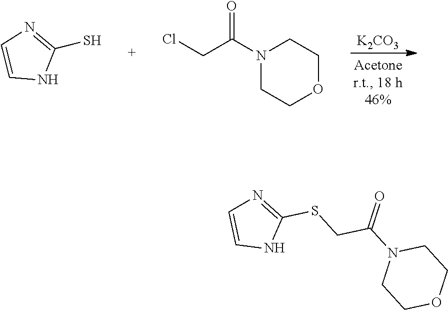

It was obtained 128 mg of the title compound as a white solid (46% yield).

### TTC-23: Synthesis of 2-(2-oxocyclohexyl)isoindoline-1,3-dione (Can. J. Chem., 1985, 63, 3186)

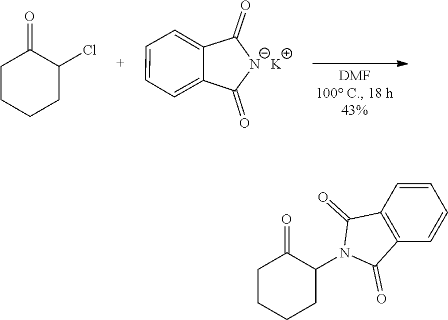

In a round-bottom flask were added 1.5 g (8.3 mmol) of potassium phthalimide and 1.0 g (7.54 mmol) of 2-chlorocyclohexanone in 7 mL of DMF. The mixture was heated under 100° C. for 18 h. After that, the reaction was poured in water and extracted with dichloromethane (5×20 mL). The organic phase was washed a saturated solution of sodium carbonate and after with brine. The organic phase was dried over with sodium sulfate and the solvent removed under reduced pressure. It was obtained a yellow solid, which was purified by column chromatography, increasing the amount of ethyl acetate of 20 to 30% in petroleum ether. It was obtained 790 mg of a yellow solid (43% yield).

### Synthesis of 4,5,6,7-tetrahydro-1H-benzo[d]imidazole-2-thiol (ChemCatChem, 2015, 7, 2206)

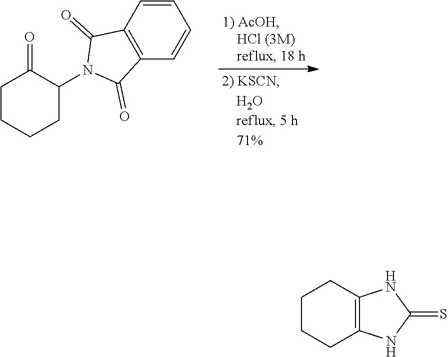 [SMILES: O=C1CCCCC1N1C(=O)c2ccccc2C1=O ; S=c1[nH]c2c([nH]1)CCCC2]

A solution of the 2-(2-oxocyclohexyl)isoindoline-1,3-dione (2 mmol) in 5 mL of AcOH and 5 mL of a solution of HCl (3M) was stirred under reflux for 18 h. The mixture was partially concentrated and it was observed the precipitation of a brown solid, the phthalic acid, which was filtrated. The solution was concentrated and it was obtained the crude intermediate, the HCl salt of the 2-aminocyclohexanone, which was dried under vacuum overnight. The intermediate was used without further purification. For the next step, the intermediate was added into 25 mL of water and 1.5 eq of KSCN. The reaction stirred under reflux for 5 h. The reaction was extracted with ethyl acetate (4×20 mL) and dried over with sodium sulfate. The solvent was removed under reduced pressure. It was obtained 225 mg of a brown solid (71% yield), which was used in the next step without further purification.

### TTC-23: Synthesis of 1-morpholino-2-((4,5,6,7-tetrahydro-1H-benzo[d]imidazol-2-yl)thio)ethan-1-one

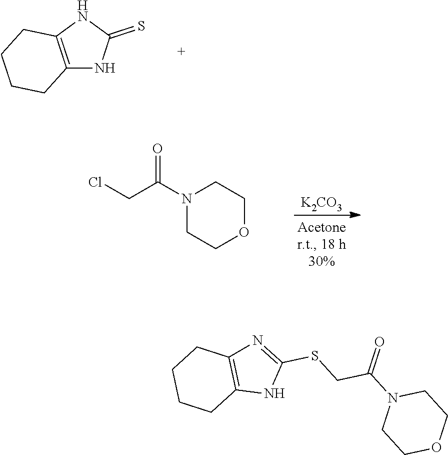

It was obtained 106 mg of the title compound as a white solid (30% yield).

General procedure for the synthesis of 3-chloro-1-morpholinopropan-1-one and Synthesis of 4-chloro-1-morpholinobutan-1-one. In a round-bottom flask were added 1 mL (11.48 mmol) of morpholine and 2 mL (13.77 mmol) of triethylamine in 75 mL of dichloromethane. The mixture was cooled to 0° C. Subsequently, a solution containing 12.62 mmol of the respective acid chloride in 20 mL dichloromethane was slowly added to the solution containing the amine. The reaction mixture was kept under stirring for 30 minutes at 0° C. The reaction was warmed to room temperature and stirred for more 2 h. After that, the mixture was washed with a solution of HCl 1M and after that with a solution of NaHCO₃ saturated. The organic phase was dried over with sodium sulphate and concentrated under reduced pressure.

### Synthesis of 3-chloro-1-morpholinopropan-1-one

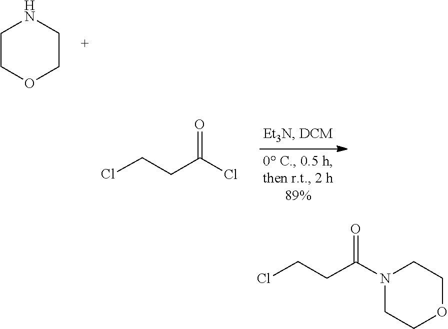 [SMILES: C1COCCN1 ; O=C(Cl)CCCl ; O=C(CCCl)N1CCOCC1]

It was obtained 1.82 g of the title compound as a yellow oil (89% yield).

### Synthesis of 4-chloro-1-morpholinobutan-1-one

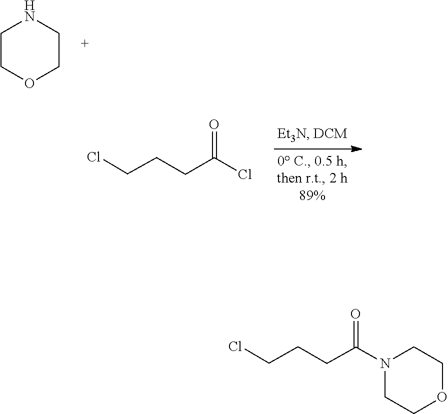 [SMILES: C1COCCN1 ; O=C(Cl)CCCCl ; O=C(CCCCl)N1CCOCC1]

It was obtained 1.97 g of the title compound as a yellow oil (89% yield).

General Procedure for the synthesis of TTC-24 and TTC-25: In a round-bottom flask were added 300 mg (1.92 mmol) of (±)-trans-octahydro-2H-benzo[d]imidazole-2-thione and 2.88 mmol of 3-chloro-1-morpholinopropan-1-one or 4-chloro-1-morpholinobutan-1-one in 10 mL of isopropanol. The reaction mixture was stirred under reflux for 72 hours. There was no formation of precipitate. The solvent was removed under reduced pressure and 25 mL of a 10% HCl solution was added. The solution was extracted with AcOEt. The aqueous phase was basified with sodium carbonate (pH=10) and extracted with AcOEt. The organic phase was dried over with Na₂SO4 and removed under reduced pressure. A white solid was formed after the complete removal of the solvent. The residue was washed with hot n-hexane to obtain the pure product as white solid.

### TTC-24: Synthesis of 3-(((3aS,7aS)-3a,4,5,6,7,7a-hexahydro-1H-benzo[d]imidazol-2-yl)thio)-1-morpholinopropan-1-one

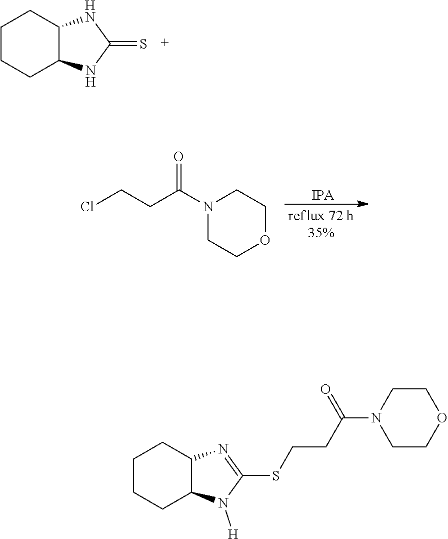

It was obtained 200 mg of the title compound as a white solid (35% yield).

### TTC-25: Synthesis of 4-(((3aS,7aS)-3a,4,5,6,7,7a-hexahydro-1H-benzo[d]imidazol-2-yl)thio)-1-morpholinobutan-1-one

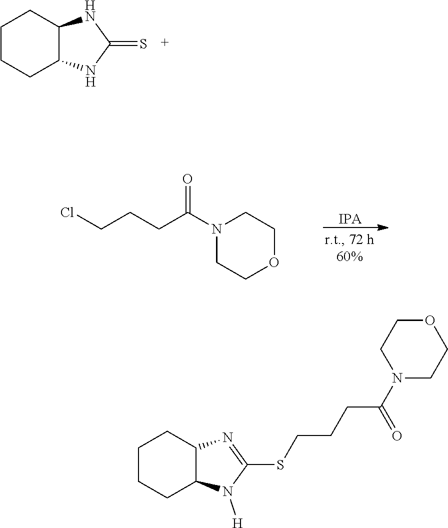

It was obtained 360 mg of the title compound as a white solid (60% yield).

### TTC-26 (R,R-TTC-14): Synthesis of (3aR,7aR)-2-((4-methylpiperidin-1-yl)-2-oxoethyl)thio)-3a,4,5,6,7,7a-hexahydro-1H-benzo[d]imidazol-3-ium chloride

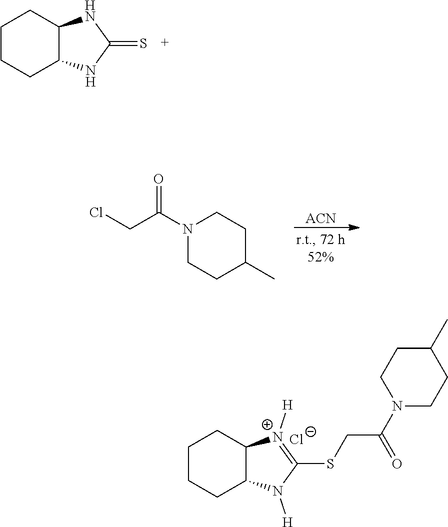

It was obtained 210 mg of the title compound as a white solid (52% yield). The spectral data is the same of the racemic mixture.

### TTC-27 (S,S-TTC-14): Synthesis of (3aS,7aS)-2-((4-methylpiperidin-1-yl)-2-oxoethyl)thio)-3a,4,5,6,7,7a-hexahydro-1H-benzo[d]imidazol-3-ium chloride

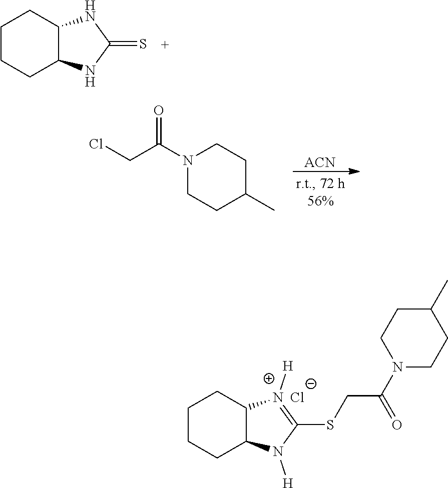

It was obtained 227 mg of the title compound as a white solid (56% yield). The spectral data is the same of the racemic mixture. Elemental analysis calculated for C₁₅H₂₆ClN₃OS: C, 54.28; H, 7.90; N, 12.66. Found C, 54.14; H, 7.87; N, 12.58.

### TTC-28: Synthesis of (±)-trans-2-((2-(4-ethylpiperidin-1-yl)-2-oxoethyl)thio)-3a,4,5,6,7,7a-hexahydro-1H-benzo[d]imidazol-3-ium chloride

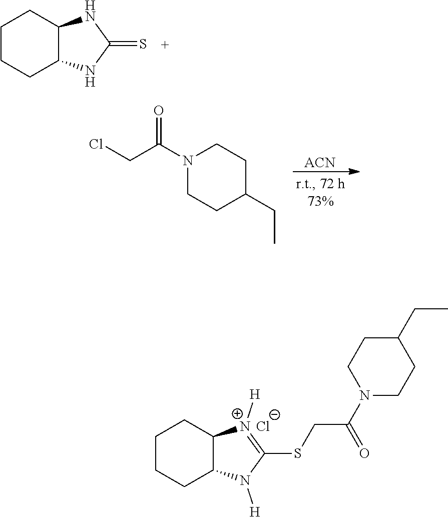

It was obtained 310 mg of the title compound (mixture of conformers) as a white solid (73% yield).

### TTC-29: Synthesis of (±)-trans-2-((2-oxo-2-(4-propylpiperidin-1-yl)ethyl)thio)-3a,4,5,6,7,7a-hexahydro-1H-benzo[d]imidazol-3-ium chloride

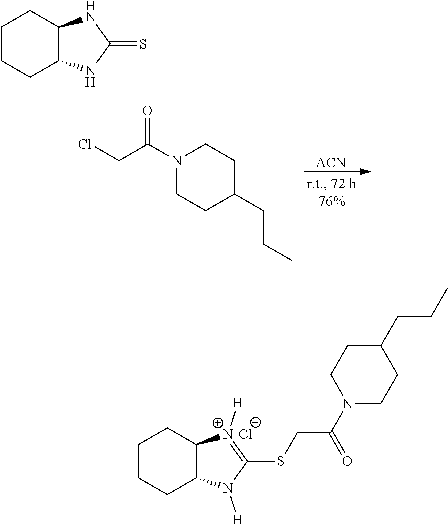 [SMILES: S=C1N[C@@H]2CCCC[C@H]2N1 ; CCCC1CCN(C(=O)CCl)CC1 ; "ACN" ; CCCC1CCN(C(=O)CSC2=[NH+][C@@H]3CCCC[C@H]3N2)CC1 ; [Cl-]]

It was obtained 336 mg of the title compound (mixture of conformers) as a white solid (76% yield).

### TTC-30: Synthesis of (±)-trans-2-((2-(4-isopropylpiperidin-1-yl)-2-oxoethyl)thio)-3a,4,5,6,7,7a-hexahydro-1H-benzo[d]imidazol-3-ium chloride

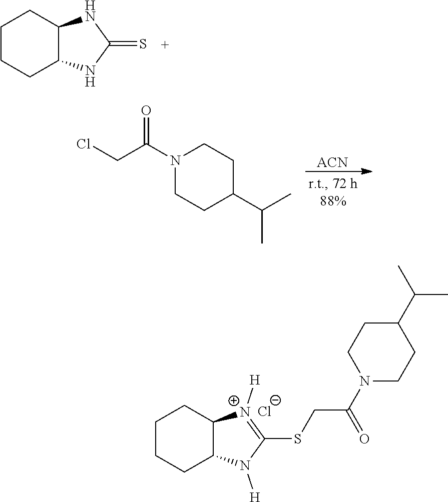

It was obtained 390 mg of the title compound (mixture of conformers) as a white solid (88% yield).

### TTC-31: Synthesis of (±)-trans-2-((2-(4-(tert-butyl)piperidin-1-yl)-2-oxoethyl)thio)-3a,4,5,6,7,7a-hexahydro-1H-benzo[d]imidazol-3-ium chloride

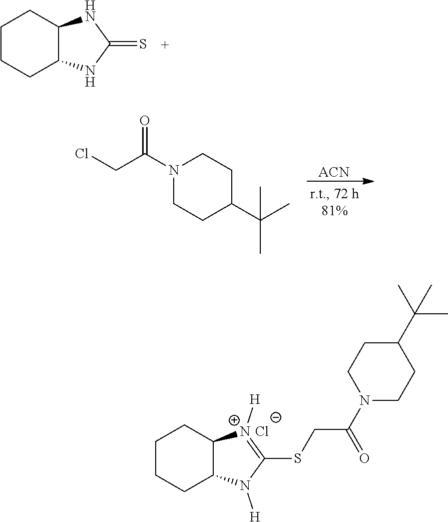

It was obtained 373 mg of the title compound (mixture of conformers) as a white solid (81% yield).

### TTC-32: Synthesis of (±)-trans-2-((2-oxo-2-(4-(trifluoromethyl)piperidin-1-yl)ethyl)thio)-3a,4,5,6,7,7a-hexahydro-1H-benzo[d]imidazol-3-ium chloride

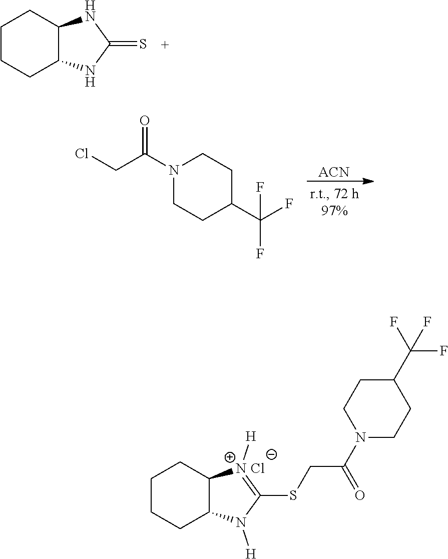

It was obtained 460 mg of the title compound (mixture of conformers) as a white solid (97% yield).

### TTC-33: Synthesis of (±)-trans-2-((2-oxo-2-(4-phenylpiperidin-1-yl)ethyl)thio)-3a,4,5,6,7,7a-hexahydro-1H-benzo[d]imidazol-3-ium chloride

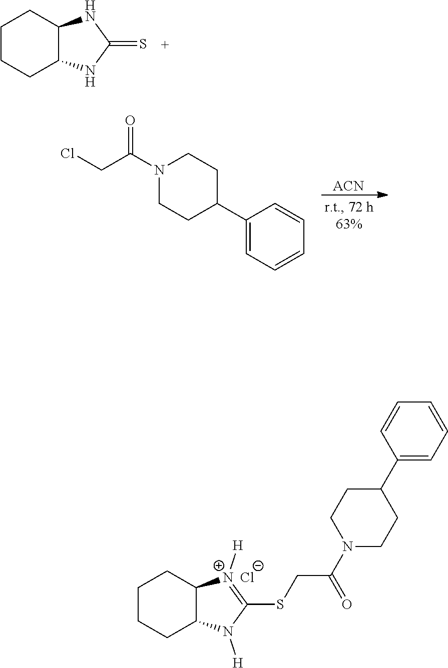

It was obtained 302 mg of the title compound (mixture of conformers) as a white solid (63% yield).

### TTC-34: Synthesis of (±)-trans-2-((2-oxo-2-(4-(3-phenylpropyl)piperidin-1-yl)ethyl)thio)-3a,4,5,6,7,7a-hexahydro-1H-benzo[d]imidazol-3-ium chloride

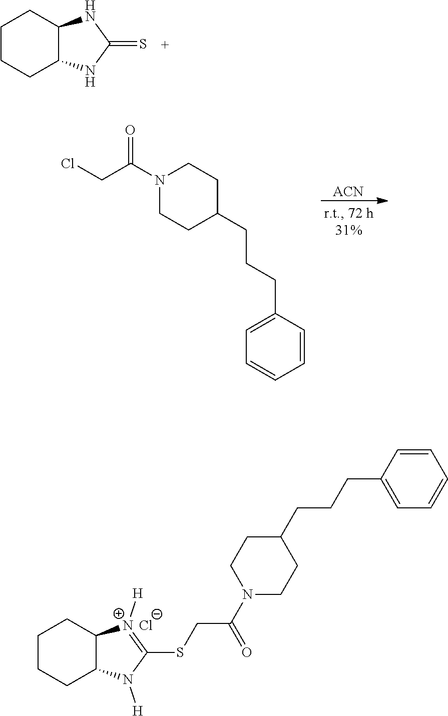 [SMILES: S=C1N[C@@H]2CCCC[C@H]2N1 ; O=C(CCl)N1CCC(CCCc2ccccc2)CC1 ; "ACN" ; O=C(CSC1=[NH+][C@@H]2CCCC[C@H]2N1)N1CCC(CCCc2ccccc2)CC1 ; [Cl-]]

It was obtained 161 mg of the title compound (mixture of conformers) as a white solid (31% yield).

### TTC-35: Synthesis of (±)-trans-2-((2-(4-(acetamidomethyl)piperidin-1-yl)-2-oxoethyl)thio)-3a,4,5,6,7,7a-hexahydro-1H-benzo[d]imidazol-3-ium chloride

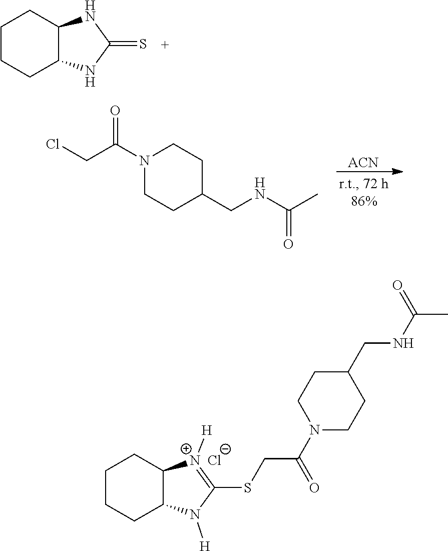

It was obtained 411 mg of the title compound (mixture of conformers) as a white solid (86% yield).

### TTC-36: Synthesis of (±)-trans-2-((2-oxo-2-(4-(o-tolyl)piperazin-1-yl)ethyl)thio)-3a,4,5,6,7,7a-hexahydro-1H-benzo[d]imidazol-3-ium chloride

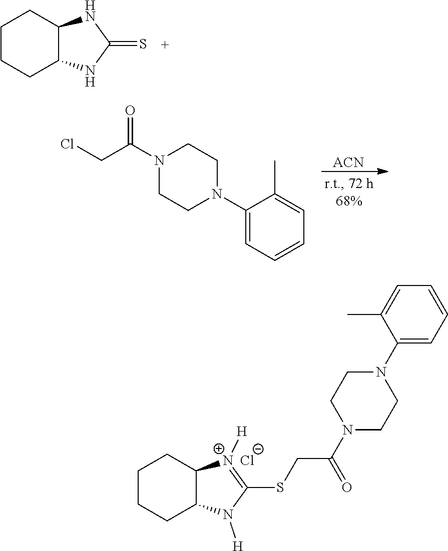

It was obtained 339 mg of the title compound as a white solid (68% yield).

### TTC-37: Synthesis of (±)-trans-2-((2-(4-(4-chlorophenyl)piperazin-1-yl)-2-oxoethyl)thio)-3a,4,5,6,7,7a-hexahydro-1H-benzo[d]imidazol-3-ium chloride

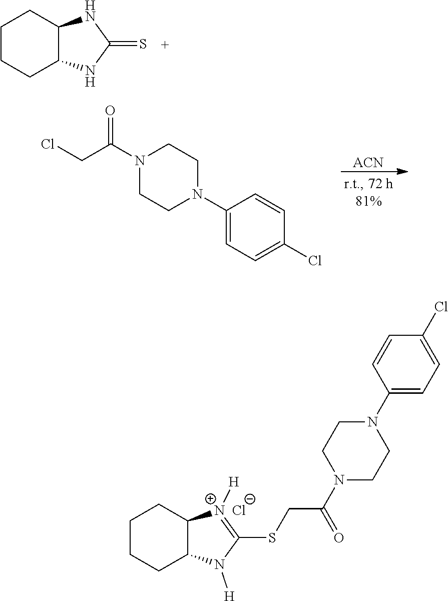

It was obtained 428 mg of the title compound as a white solid (81% yield).

### TTC-38: Synthesis of (±)-trans-2-((2-oxo-2-(4-(p-tolyl)piperazin-1-yl)ethyl)thio)-3a,4,5,6,7,7a-hexahydro-1H-benzo[d]imidazol-3-ium chloride

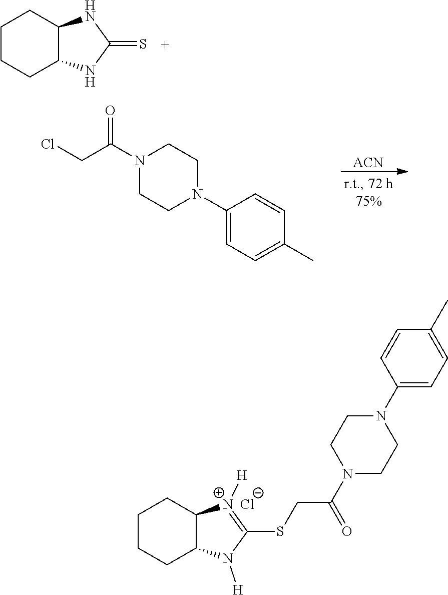

It was obtained 376 mg of the title compound as a white solid (75% yield).

### TTC-39: Synthesis of (±)-trans-2-((2-oxo-2-(4-(4-(trifluoromethyl)phenyl)piperazin-1-yl)ethyl)thio)-3a,4,5,6,7,7a-hexahydro-1H-benzo[d]imidazol-3-ium chloride

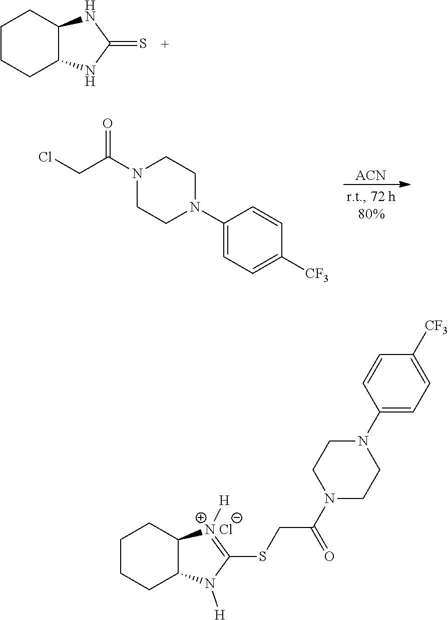 [SMILES: S=C1N[C@@H]2CCCC[C@H]2N1 ; O=C(CCl)N1CCN(c2ccc(C(F)(F)F)cc2)CC1 ; "ACN" ; O=C(CSC1=[NH+][C@@H]2CCCC[C@H]2N1)N1CCN(c2ccc(C(F)(F)F)cc2)CC1 ; [Cl-]]

It was obtained 455 mg of the title compound as a white solid (80% yield).

### TTC-40: Synthesis of (±)-trans-2-((-(4-acetylpiperazin-1-yl)-2-oxoethyl)thio)-3a,4,5,6,7,7a-hexahydro-1H-benzo[d]imidazol-3-ium chloride

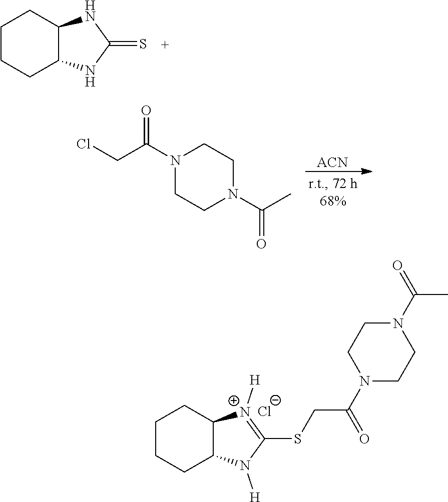

It was obtained 300 mg of the title compound (mixture of conformers) as a white solid (68% yield).

### TTC-41: Synthesis of (±)-trans-2-((2-(4-benzoylpiperazin-1-yl)-2-oxoethyl)thio)-3a,4,5,6,7,7a-hexahydro-1H-benzo[d]imidazol-3-ium chloride

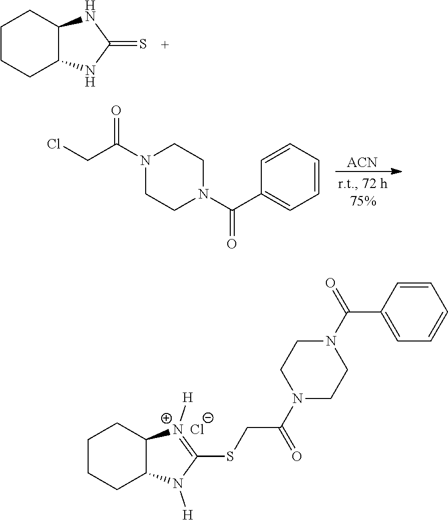

It was obtained 390 mg of the title compound as a white solid (75% yield).

### Evaluation of the TTC Series

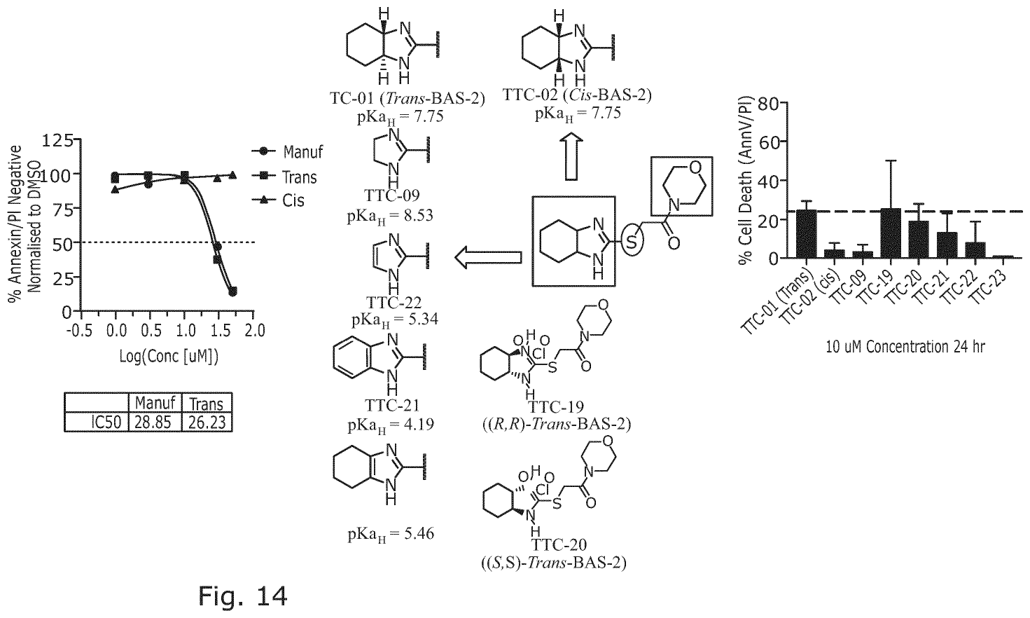

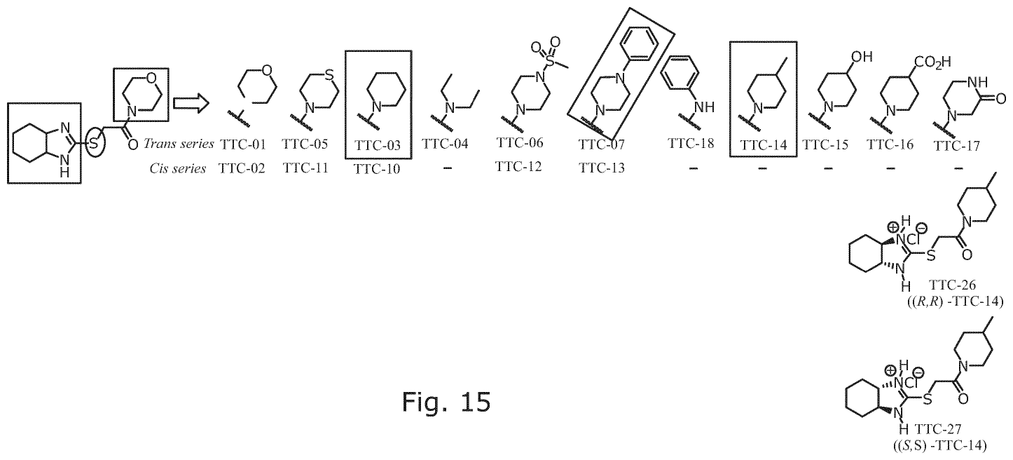

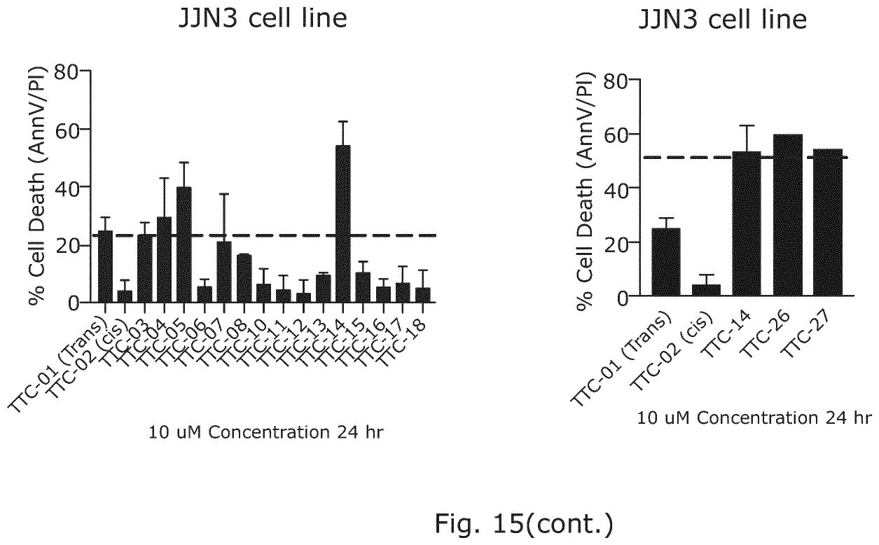

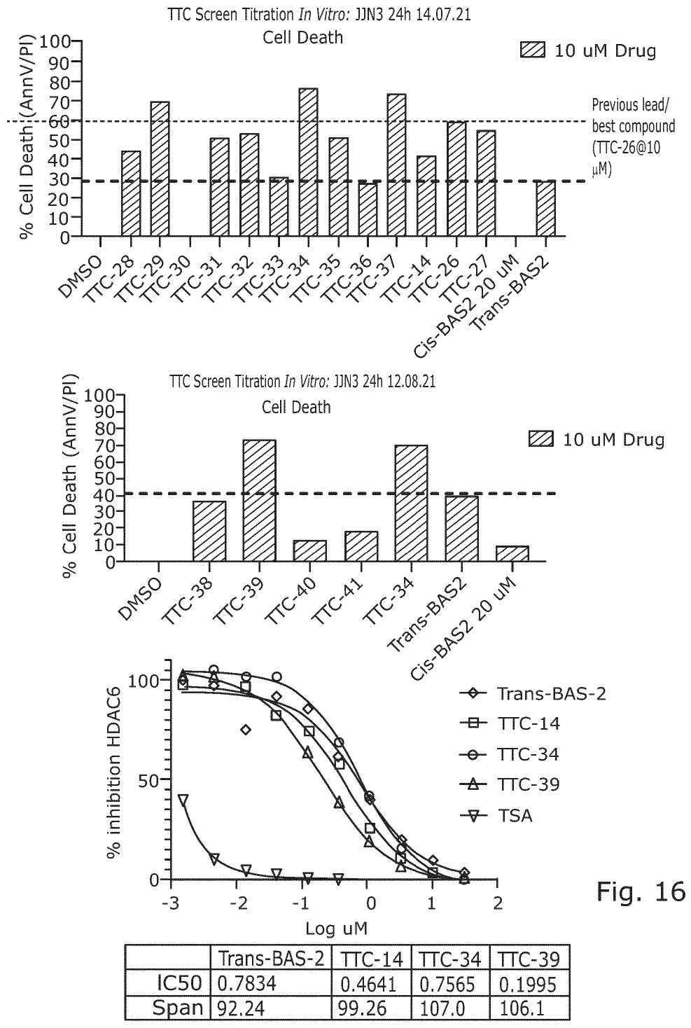

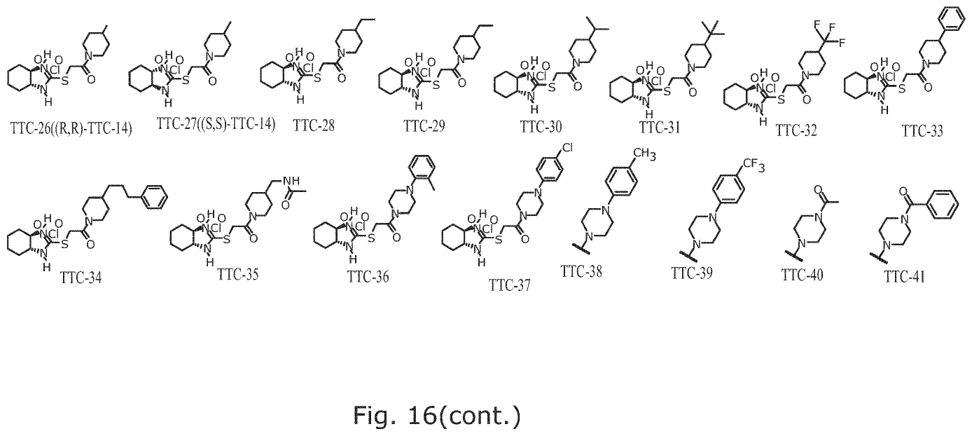

The compounds were evaluated using the multiple myeloma JJN3 cell line, the activity is showed as higher or lower than 10 μM. In addition, 4 compounds were selected for the determination of HDAC6 inhibition. The evaluation of the compounds are showed in the FIGS. 14, 15 and 16.

TABLE
Apoptosis assay using JJN3 cell line and 24 h of treatment.

| Compounds | Activity (IC₅₀) |
|---|---|
| TTC-01 | >10 μM |
| TTC-02 | >10 μM |
| TTC-03 | >10 μM |
| TTC-04 | >10 μM |
| TTC-05 | <10 μM |
| TTC-06 | >10 μM |
| TTC-07 | >10 μM |
| TTC-08 | >10 μM |
| TTC-09 | >10 μM |
| TTC-10 | >10 μM |
| TTC-11 | >10 μM |
| TTC-12 | >10 μM |
| TTC-13 | >10 μM |
| TTC-14 | <10 μM |
| TTC-15 | >10 μM |
| TTC-16 | >10 μM |
| TTC-17 | >10 μM |
| TTC-18 | >10 μM |
| TTC-19 | >10 μM |
| TTC-20 | >10 μM |
| TTC-21 | >10 μM |
| TTC-22 | >10 μM |
| TTC-23 | >10 μM |
| TTC-24 | >10 μM |
| TTC-25 | >10 μM |
| TTC-26 | <10 μM |
| TTC-27 | <10 μM |
| TTC-28 | >10 μM |
| TTC-29 | <10 μM |
| TTC-30 | >10 μM |
| TTC-31 | <10 μM |
| TTC-32 | <10 μM |
| TTC-33 | >10 μM |
| TTC-34 | <10 μM |
| TTC-35 | <10 μM |
| TTC-36 | >10 μM |
| TTC-37 | <10 μM |
| TTC-38 | >10 μM |
| TTC-39 | <10 μM |
| TTC-40 | >10 μM |
| TTC-41 | >10 μM |

TABLE
HDAC6 inhibition.

| Compound | IC₅₀ (nM) |
|---|---|
| TTC-01 | 783 |
| TTC-14 | 464 |
| TTC-34 | 756 |
| TTC-39 | 199 |

What is claimed is:

1. A method of treating a subject in need thereof, the method comprising the step of administering, to a patient, a compound according to formula I or a pharmaceutically acceptable salt, solvate, ester or pro-drug thereof, 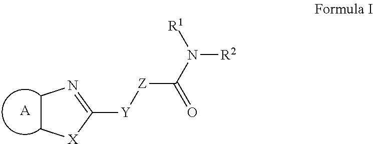
wherein:
either (i) NR¹R² together forms a 5- or 6-membered heterocyclic ring or (ii) R¹ and R² are independently selected from H and C1 to C12 alkyl;
A is a non-aromatic ring, an aromatic ring or a double bond;
X is NR³, S or O, where R³ is selected from H and C1 to C12 alkyl;
Y is S, NR⁴, CR⁴R⁵, or O, where R⁴ and R⁵ are independently selected from H and C1 to C12 alkyl; and
Z is (CR⁶R⁷)ₙ where R⁶ and R⁷ are independently selected from H and C1 to C12 alkyl and n is an integer from 1 to 6.

2. The method according to claim 1, wherein A is a non-aromatic ring or an aromatic ring.

3. The method according to claim 1, wherein A forms a double bond.

4. The method according to claim 1, wherein X is NR³, such as NH.

5. The method according to claim 1, wherein X is S or O.

6. The method according to claim 1, wherein Y is S.

7. The method according to claim 1, wherein Y is NR⁴, CR⁴R⁵ or O.

8. The method according to claim 1, wherein (i) R⁶ is H; (ii) R⁷ is H; and/or (iii) n is 1 or 2.

9. The method according to claim 1, wherein NR¹R² together forms a 5- or 6-membered heterocyclic ring, or wherein the 5- or 6-membered heterocyclic ring comprises 1 N atom and at least one additional heteroatom selected from N, S and O.

10. (canceled)

11. The method according to claim 9, wherein the 5- or 6-membered heterocyclic ring comprises a morpholine group, thiomorpholine group, a piperidine group, a piperazine group, an oxazepane group or a thiazepane group.

12. The method according to claim 1, wherein
(i) NR¹R² together forms a morpholine group;
(ii) A is a non-aromatic ring;
(iii) X is NR³;
(iv) Y is S; and/or
(v) Z is —CH₂—.

13. The method according to claim 12, which is 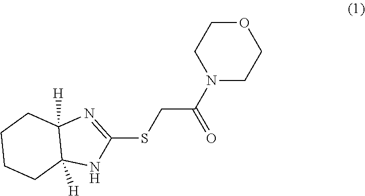 [SMILES: O=C(CSC1=N[C@@H]2CCCC[C@@H]2N1)N1CCOCC1]
or a pharmaceutically acceptable salt, solvate, ester or pro-drug thereof.

14. The method according to claim 1, wherein the method is a method of treating cancer, neurodegenerative diseases and/or inflammation.

15. The method as defined in claim 1, wherein the method is a method of regulating glycolytic metabolism.

16. The method according to claim 15, wherein the compound is combined with a metabolic inhibitor.

17. (canceled)

18. A compound which is according to formula I or is a pharmaceutically acceptable salt, solvate, ester or pro-drug thereof, 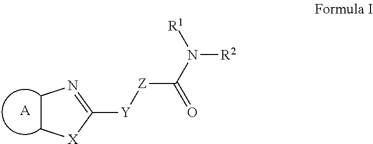
wherein
either (i) NR¹R² together forms a 5- or 6-membered heterocyclic ring or (ii) R¹ and R² are independently selected from H and C1 to C12 alkyl;
A is a non-aromatic ring, an aromatic ring or a double bond;
X is NR³, S or O, where R³ is selected from H and C1 to C12 alkyl;
Y is S, NR⁴, CR⁴R⁵, or O, where R⁴ and R⁵ are independently selected from H and C1 to C12 alkyl; and
Z is (CR⁶R⁷)ₙ where R⁶ and R⁷ are independently selected from H and C1 to C12 alkyl and n is an integer from 1 to 6.

19. The compound of claim 18, which is a compound of formula II or a pharmaceutically acceptable salt, solvate, ester or pro-drug thereof 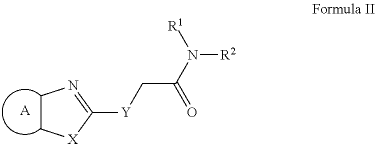
wherein
either (i) NR¹R² together forms a 5- or 6-membered heterocyclic ring or (ii) R¹ and R² are independently selected from H and C1 to C12 alkyl;
A is a non-aromatic ring, an aromatic ring or a double bond;
X is NR³, S or O, where R³ is selected from H and C1 to C12 alkyl; and
Y is S, NR⁴, CR⁴R⁵, or O, where R⁴ and R⁵ are independently selected from H and C1 to C12 alkyl.

20. The compound of claim 18, having a structure as shown herein and selected from S2 to S7, S10, S17, S19, S20, S22 to S28, C1 to C7, C15 to C28, N1 to N8, N10, N13, N15 to N18, N20 to N28, or a pharmaceutically acceptable salt, solvate, ester or pro-drug thereof.

21. The compound of claim 18, having a structure as shown below: 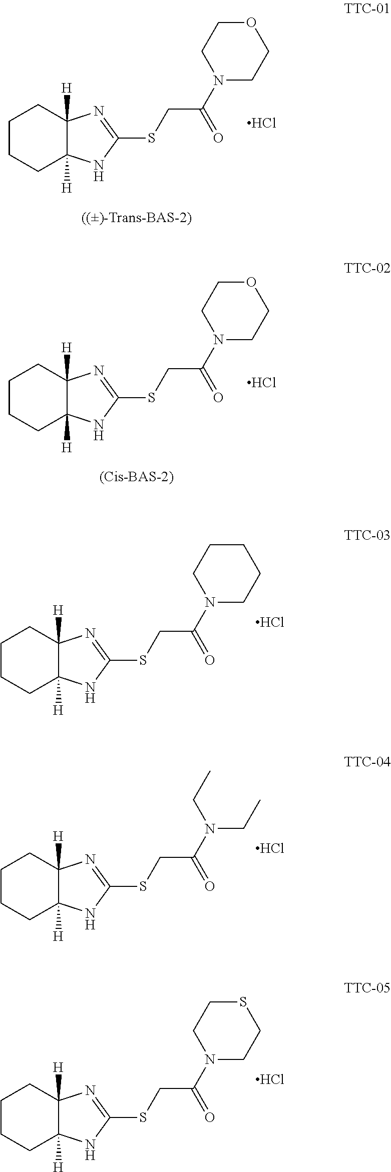 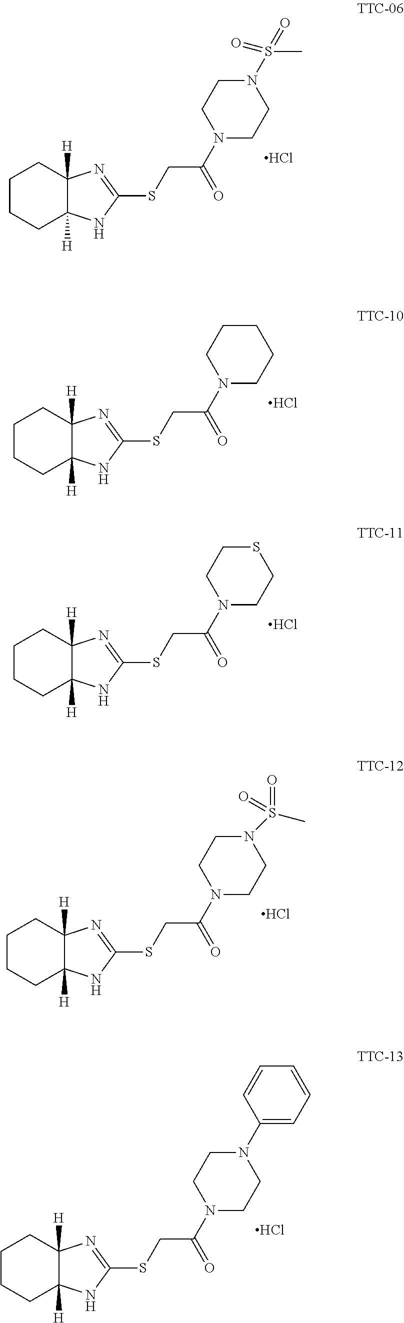 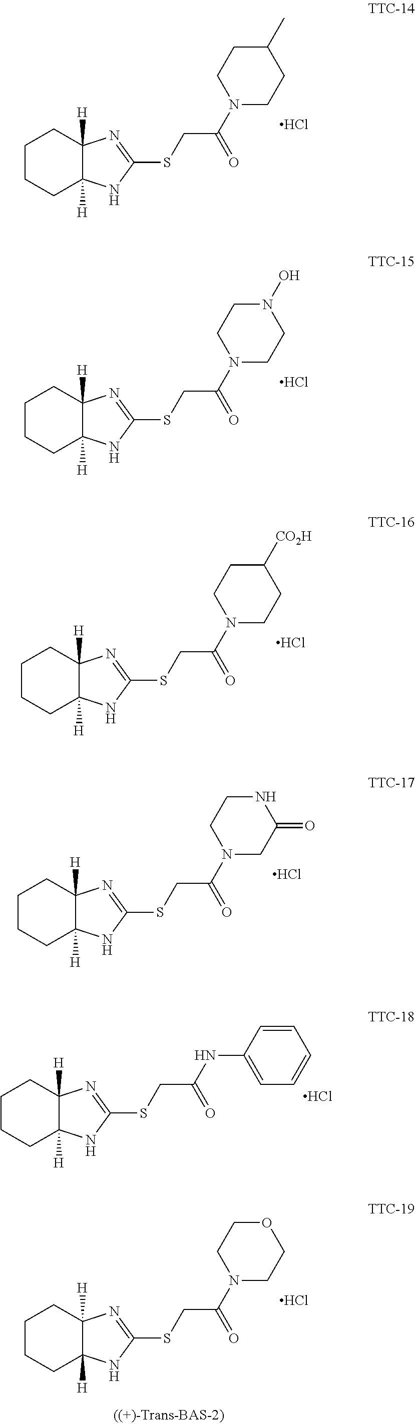 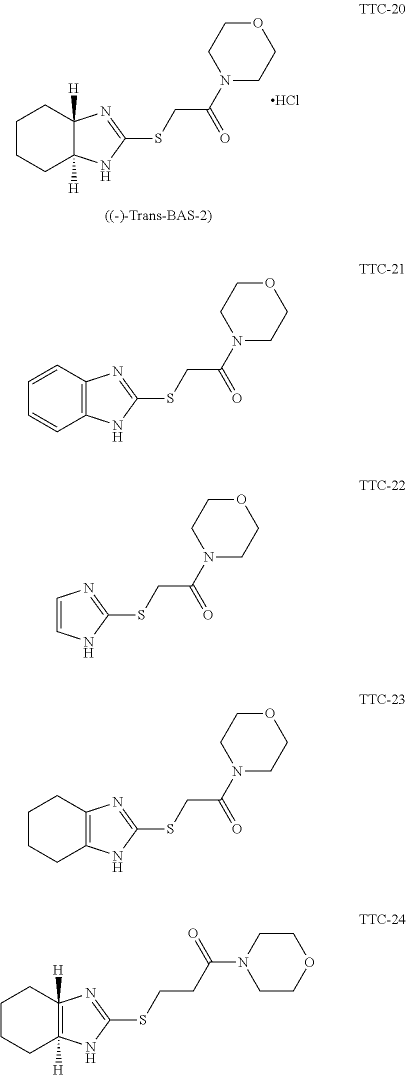 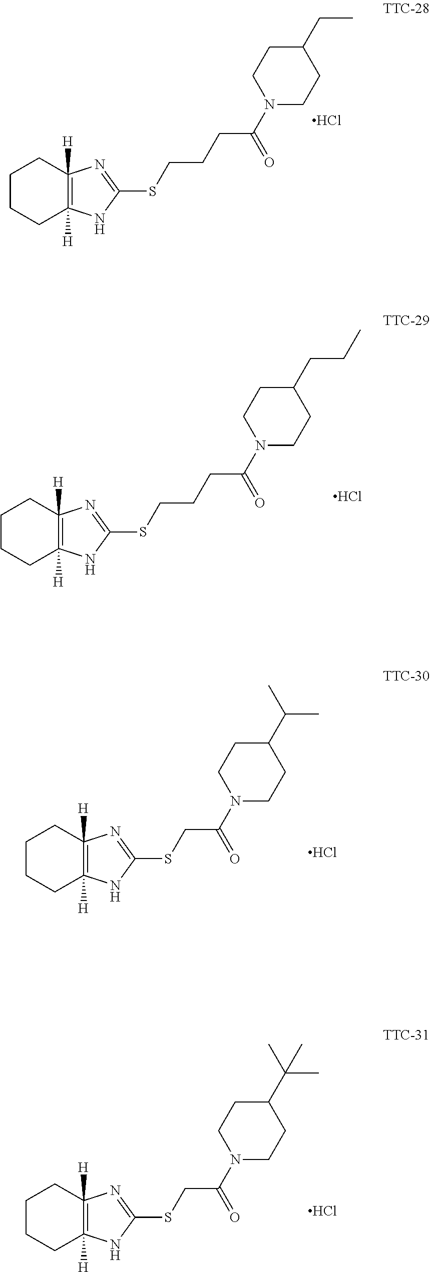 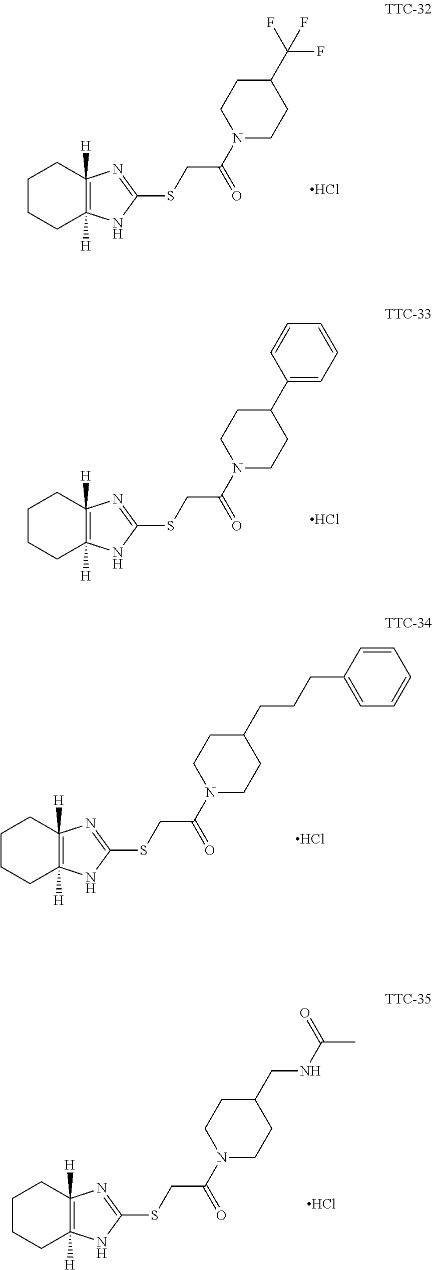 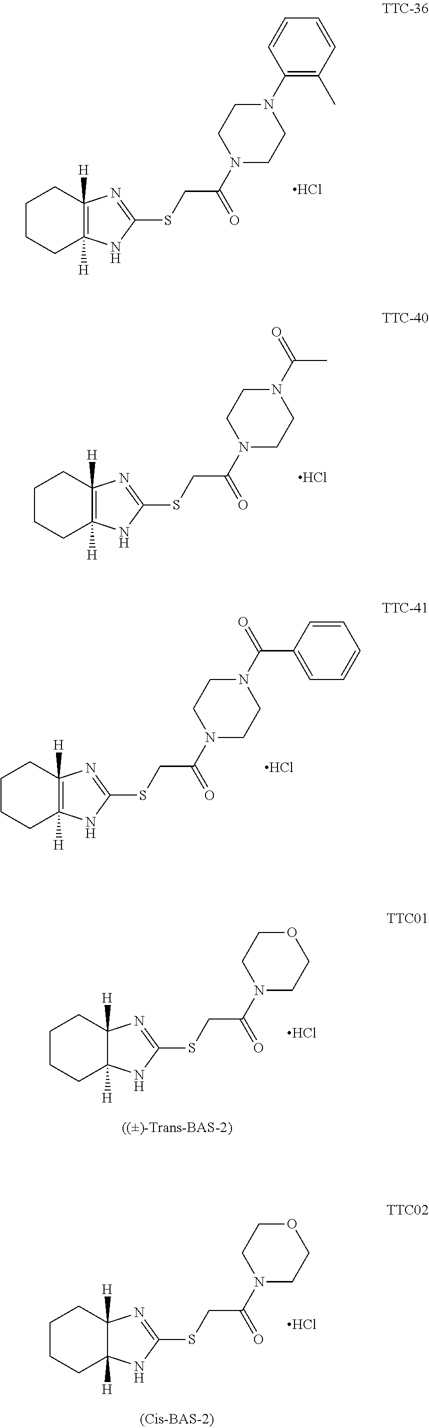 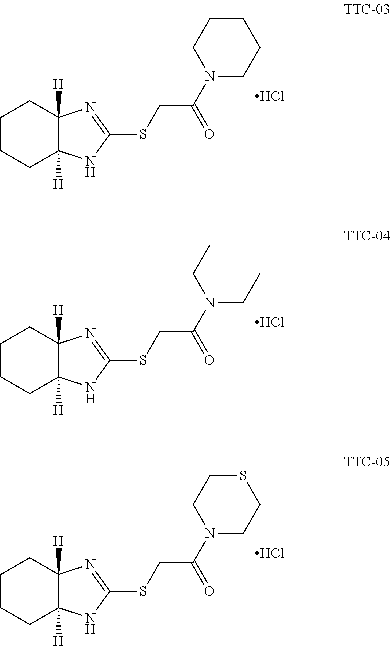 [SMILES: O=C(CSC1=N[C@H]2CCCC[C@@H]2N1)N1CCCCC1 ; Cl ; CCN(CC)C(=O)CSC1=N[C@H]2CCCC[C@@H]2N1 ; Cl ; O=C(CSC1=N[C@H]2CCCC[C@@H]2N1)N1CCSCC1 ; Cl] 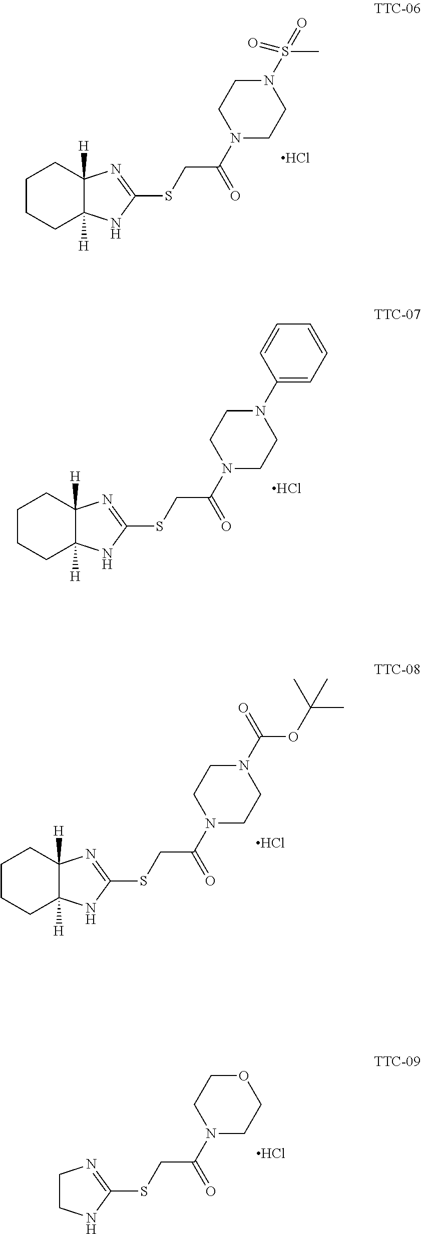 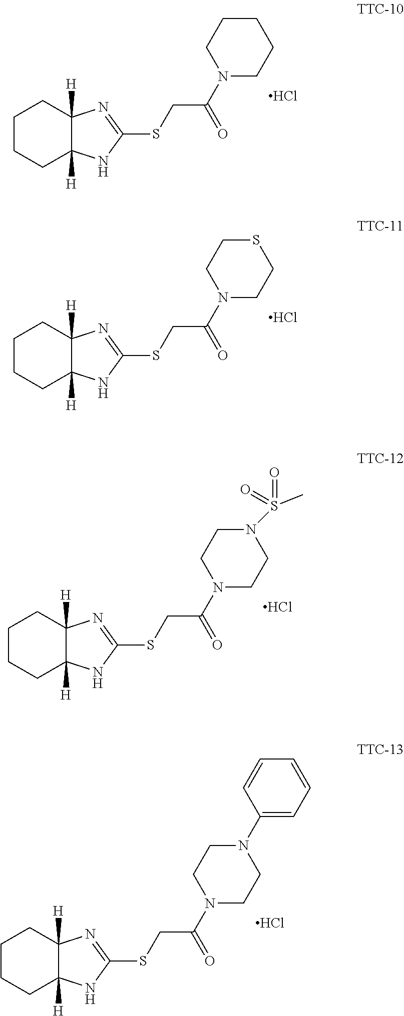 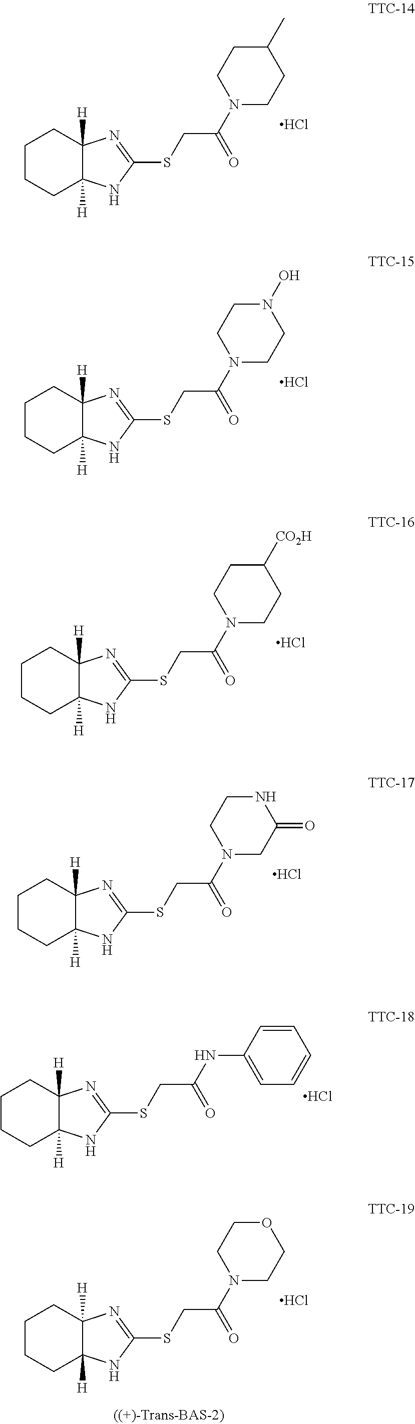 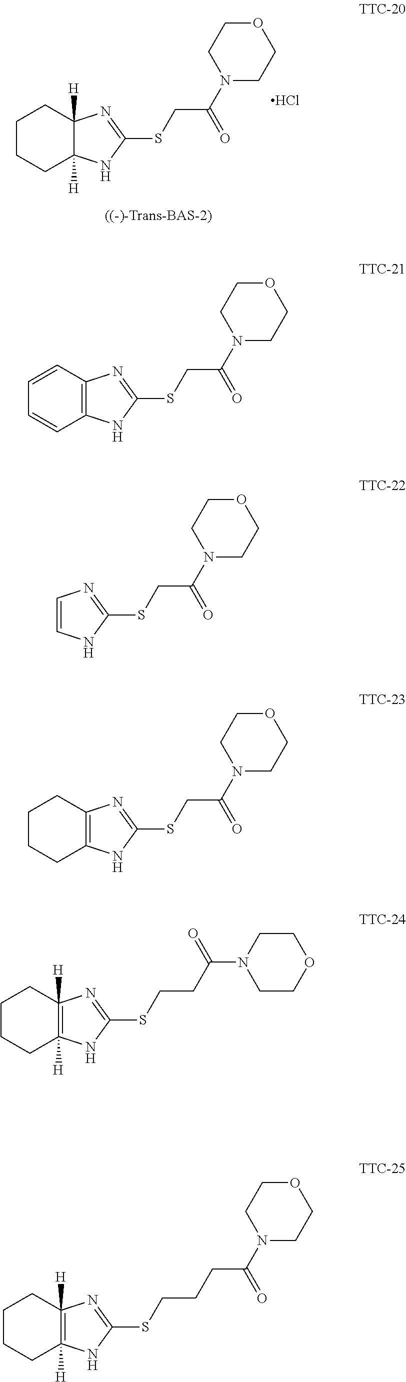 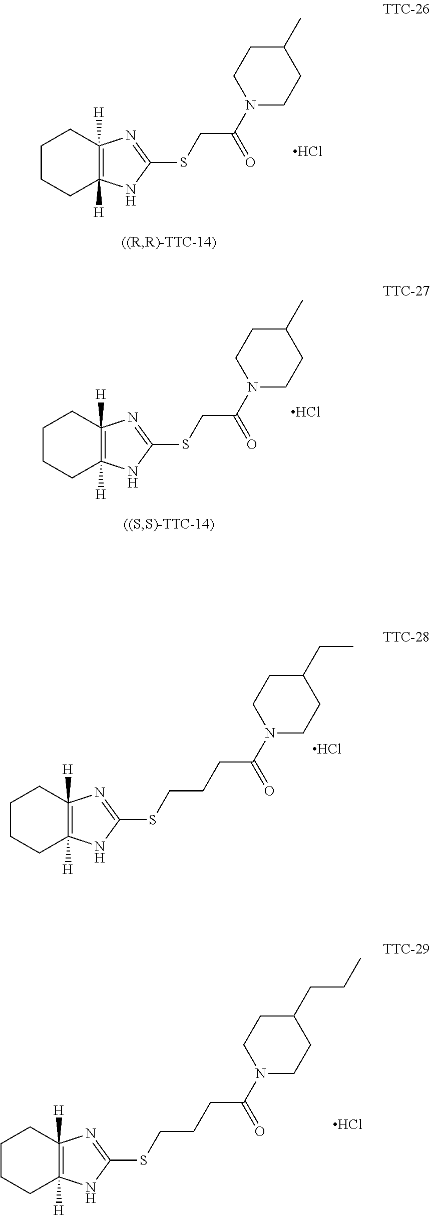 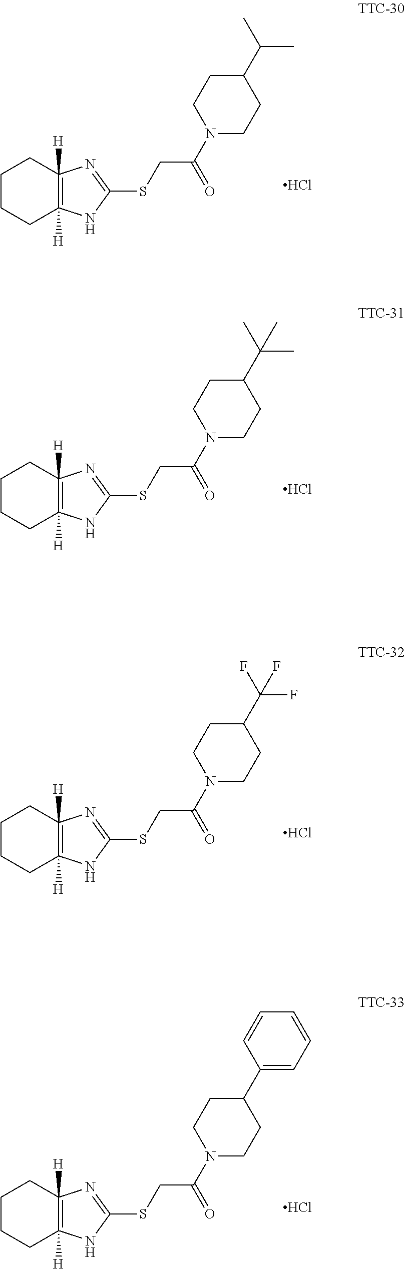 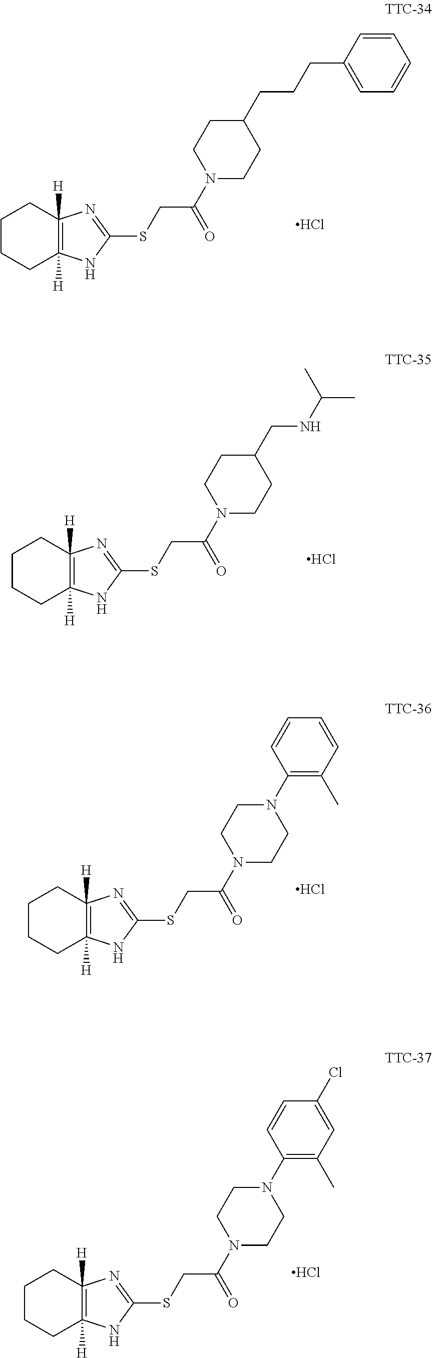 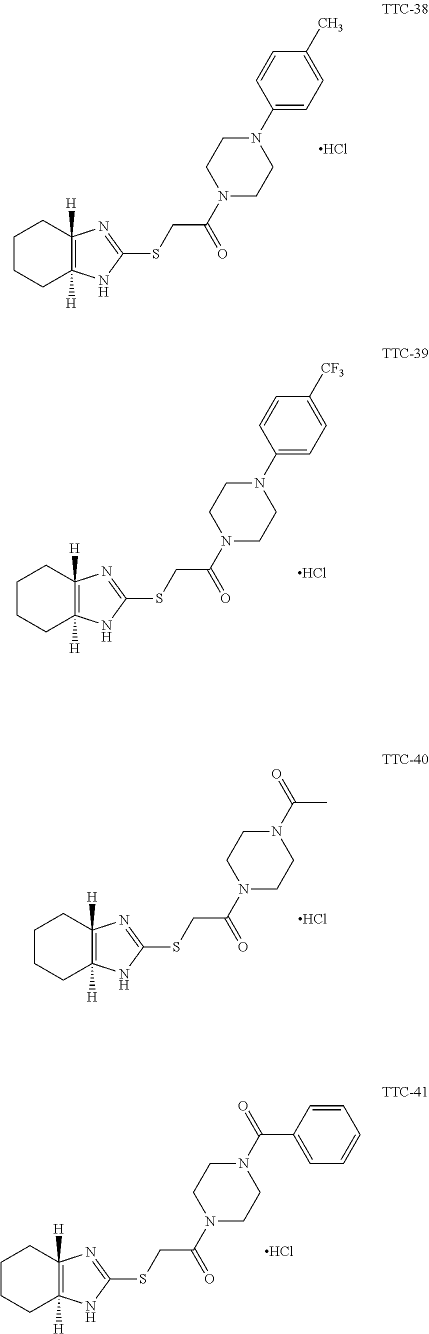
or a pharmaceutically acceptable salt, solvate, ester or pro-drug thereof.

22. A pharmaceutical composition comprising a compound as defined in claim 18 and a pharmaceutically acceptable carrier.
